(19) United States
(12) Patent Application Publication
(10) Pub. No.: US 2013/0310585 A1
(43) Pub. Date: Nov. 21, 2013

(54) METHOD FOR INCREASING PLANT OIL PRODUCTION

(71) Applicants: Xuemin Wang, St. Louis, MO (US); Maoyin Li, St. Louis, MO (US); Geliang Wang, St. Louis, MO (US)

(72) Inventors: Xuemin Wang, St. Louis, MO (US); Maoyin Li, St. Louis, MO (US); Geliang Wang, St. Louis, MO (US)

(73) Assignees: DONALD DANFORTH PLANT SCIENCE CENTER, St. Louis, MO (US); THE CURATORS OF UNIVERSITY OF MISSOURI-ST. LOUIS, St. Louis, MO (US)

(21) Appl. No.: 13/880,605

(22) PCT Filed: Oct. 19, 2011
(86) PCT No.: PCT/US2011/056861

§ 371 Date: Aug. 6, 2013

Related U.S. Application Data
(60) Provisional application No. 61/455,345, filed on Oct. 19, 2010.

(51) Int. Cl. C12N 15/82

(52) U.S. Cl. CPC C12N 15/8247
USPC 554/1; 435/468; 800/281; 800/298; 800/312

(57) ABSTRACT

The present invention provides a new and improved method for increasing plant seed oil content by seed-specific manipulation of PLD ζ expression The inventive method is applicable to a variety of plant species, such as Arabidopsis, camelina, and soybean, and has the potential to increase seed oil content (both dietary and industrial) and vegetable oil production in crops.

### CROSS REFERENCE TO RELATED APPLICATIONS

This application claims the benefit of priority of U.S. provisional application No. 61/455,345, filed Oct. 19, 2010, the disclosure of which is incorporated by reference as if written herein in its entirety.

### STATEMENT REGARDING FEDERALLY SPONSORED RESEARCH OR DEVELOPMENT

This invention was made with government support under grant number USDA 2007-35318-18393 by the Department of Agriculture. The government has certain rights in the invention.

### BACKGROUND OF THE INVENTION

### Field of the Invention

This invention relates to methods for increasing plant oil production, and transgenic plants over expressing specific phospholipase D enzymes characterized by increased oil content.

Vegetable oils are major commodities for food and feed, and have increasingly become an important source for biofuels and renewable industrial uses. However, inadequate supply of plant oils is a major challenge to broadening their biofuel and industrial applications. In most plant species storage lipids accumulate primarily in the form of triacylglycerol (TAG). TAG in plant seeds is synthesized primarily by the Kennedy pathway, which involves the action of three acyltransferases: glycerol-3-phosphate acyltransferase (GPAT), lysophosphatidic acid acyltransferase (LPAT), and diacylglycerol acyltransferase (DGAT). Genes encoding GPAT, LPAT, and DGAT, have been reported to positively influence oil content. Increased expression of acetyl-CoA carboxylase and glycerol-3-phosphate dehydrogenase have also been implicated in increasing oil content in oil seed rape. Expression of an LPAT from yeast in rapeseeds resulted in a modest increase in seed oil content in the field. Soybean expressing the yeast LPAT showed a maximal increase of 3.2% in oil content. Soy seeds expressing a DGAT 2A from the soil fungus Umbelopsis ramanniana resulted in a 1.5% increase in oil levels. Accordingly there remains a significant unmet need to create transgenic plants that exhibit significantly enhanced seed oil content.

Phospholipids, especially phosphatidic acid (PA) and phosphatidylcholine (PC), play pivotal roles in TAG production. PC serves as a substrate for fatty acid desaturation and other modifications and can also provide fatty acids directly to diacylglycerol (DAG) for TAG synthesis. A recent analysis indicates that PC contributes most DAG that is used for TAG formation in soybean. On the other hand, PA provides DAG for PC and TAG biosynthesis. PA also plays a role in acyl trafficking from plastids, where fatty acids are synthesized, to the endoplasmic reticulum (ER), where TAG is produced. Furthermore, PA has emerged as an important class of messengers in cell signaling, membrane trafficking, and cytoskeleton rearrangement.

According to prior studies, PLD is a multi-gene family of enzymes that hydrolyze phospholipids to produce PA. For example, the Arabidopsis genome has 12 identified PLDs that are classified into two subfamilies based on their protein domain structures, C2-PLDs and PX/PH-PLDs. Ten PLDs, α(3), β(2), γ(3), δ, and ε contain the Ca²⁺-dependent phospholipid-binding C2 domain, whereas PLDζ1 (zeta 1) and ζ2 (zeta 2) have N-terminal phox homology (PX) and pleckstrin homology (PH) domains. The C2-PLDs use various phospholipids as substrates whereas PLDζ hydrolyzes PC specifically. The expression of PLDζ1 is regulated by the homeobox gene, GLABRA 2 (GL2), which binds to the promoter region of PLDζ1 and suppresses its expression, and ablation of GL2 significantly increased Arabidopsis seed oil content.

However, the complex interplay of phospholipid regulation, both at the enzymatic and gene expression levels, as well as the established role of PA as a signal transduction molecule in its right, makes an a priori prediction as to the influence of over expression of phospholipase in general, and the specific issue of whether perturbation of PC and PA metabolism by PLD zeta 1 and 2 would affect overall seed oil accumulation challenging. Camelina sativa is an oilseed plant that has been little exploited in agriculture. It is similar in appearance to oilseed rape and similar in genetic characteristics to Arabidopsis thaliana. As Arabidopsis, it can be readily transformed by floral dip. Camelina is not a foodstuff plant and grows on marginal lands that are generally considered unsuitable for large scale food production. Camelina is being investigated as a winter crop for southern Missouri and could potentially be double-cropped with soy. These characteristics make Camelina an ideal candidate plant to be developed as a chemical factory, particularly if high level production and accumulation of chemicals can be demonstrated in seeds.

The current invention is based, at least in part, on the surprising discovery that the over expression of phospholipase Ds zeta 1 and zeta 2, in Camelina, and other plant seeds results in the high level accumulation of various triacylglycerols within the seeds. Without being limited to any particular theory of operation, it is believed that the over expression of PLD zeta 1 and zeta 2 results in the stimulated production of phosphatic acid, which stimulates the expression of the key lipid synthetic genes, including AAPT (aminoalcoholphosphotransferase) to up-regulate overall lipid accumulation in the plant, and specifically in the seeds. The resulting transgenic plants provide for an improved approach for the large scale commercial production of commercially important seed oils in plants, with the potential to directly provide a renewable source of hydrocarbons, suitable for use for the production of fuels, organic solvents, plastics and high value industrial raw materials.

### SUMMARY OF INVENTION

In one embodiment the current invention includes a method for increasing plant seed oil content comprising the steps of: 1) providing a plant seed, and 2) overexpressing one or multiple enzymes of the PLD zeta family in the seed under the control of expression control elements that drives PLD zeta expression in seeds. In one aspect of this method, the PLD zeta enzyme is selected from one or more enzymes listed in Table D1. In some embodiments of these methods the expression control elements comprise a promoter selected from the β-conglycinin promoter, oleosin promoter, and napin promoter. In some embodiments of these methods, the plant seed is selected from Arabidopsis, camelina and soybean.

In one embodiment the current invention includes a method for the production of a seed oil, comprising the step of: 1) transforming a plant cell with a nucleotide sequence encoding a PLD ζ operatively linked to expression control sequences that drive expression of the PLD ζ in the plant cell. In some aspects of this method, the amino acid sequence of the PLD ζ is selected from Table D1. In some aspects of this method, the method includes the further step of; 2) comprising regenerating stably transformed transgenic plants.

In some aspects of any of these methods, the expression control sequences comprise a cell type specific promoter. In some aspects the expression control sequences comprise a seed specific promoter. In some aspects the seed specific promoter is selected from the group consisting of soybean oleosin promoter, the rapeseed napin promoter and beta conglycinin promoter. In some aspects, plant cell is derived from a monocotyledonous plant. In some aspects, the plant cell is derived from a dicotyledonous plant. In some aspects, the plant cell is derived from Camelina. In some aspects, the plant cell is derived from Arabidopsis. In some aspects, the plant cell is derived from soybean. In some aspects of any of these methods, the method further comprises the step of growing the transgenic plant, and harvesting the seeds.

In one embodiment the current invention includes transgenic plant comprising within its genome, a nucleotide sequence encoding a protein comprising a PLD ζ operatively linked to expression control sequences that drive expression of the PLD ζ in the plant cell; wherein the PLD ζ is expressed primarily in the plant seeds. In some embodiments, the PLD ζ expression is increased compared to the corresponding wild type plant. In some embodiments the nucleotide sequence encoding a protein comprising a PLD ζ is heterologous. In some embodiments, the amino acid sequence of the PLD ζ is selected from Table D1. In some embodiments, the expression control sequences comprise a cell type specific promoter. In some embodiments, the expression control sequences comprise a seed specific promoter. In some embodiments, the seed specific promoter is selected from the group consisting of an oleosin promoter, a napin promoter and a beta conglycinin promoter. In some embodiments, the transgenic plant is derived from a monocotyledonous plant. In some embodiments, the transgenic plant is derived from a dicotyledonous plant. In some embodiments, the transgenic plant is derived from Camelina sativa. In some embodiments, the transgenic plant is derived from Arabidopsis. In some embodiments, the transgenic plant is derived from soybean.

In some embodiments, of any of these methods and transgenic plants, the transgenic plant is characterized by having an increased seed oil content compared to a corresponding wild type organism grown under similar conditions. In some embodiments, of any of these methods and transgenic plants the transgenic plant is characterized by having an increase in the relative levels (mol %) of linoleic (18:2), linolenic (18:3), and gondoic (20:1) fatty acids when compared to a corresponding wild type organism grown under similar conditions.

### DESCRIPTION OF DRAWINGS

FIG. 1 shows the impact of over expression of PLDζ1 and ζ2 in Arabidopsis on seed yield (mg) in WT and high-oil PLDζ over expressing transgenic lines. (a) shows the relative expression of PLDζ1 and ζ2 expression in seeds of WT (Col), pldζ1ζ2 double KO, PLDζ1-Over Expresser (OE) (line 3-4), and PLD ζ2-OE (line 4-3) cell lines as quantified by real-time PCR using RNA from developing seeds. The transcript level of PLDζ1 and ζ2 was expressed relative to that of UBQ10. Values are mean±SD (n=3). *P<0.05 difference from WT seeds by Student's t test. (b) Total seed yield (mg) per plant from T3 generation. Values are mean±SD (n=5).

FIG. 2 Shows the seed oil content in PLDζ1- and ζ2-altered Arabidopsis. (a) Oil content of PLDζ1 and ζ2 single and double knockout mutants in seeds in Col and WS ecotypes. Values are means±SD (n=5). (b) Immunoblotting of PLDζ1 or 2 in developing siliques in PLDζ-Over Expresser (OE) transgenic lines. Proteins (20 μg/lane) from siliques (15 days post-flowering) were separated by SDS-PAGE followed by blotting with anti-flag antibodies. (c) Oil content in PLDζ-OE lines. Values are means±SD (n=3 for each transgenic line; n=6 for WT). *P<0.05 significant difference from corresponding WT seeds by Student's t test.

FIG. 3 Shows the impact of changing the expression of PLDζ on fatty acid composition of seed oils in Arabidopsis. Lipids were extracted from dry seeds and transmethylated, followed by gas chromatography analysis. (a) Fatty acid composition in seed oils of Col-0 WT, pldζ1-KO, pldζ2-KO, and pldζ1ζ2-KO. (b) Fatty acid composition in seed oils of WT, PLD ζ1-Over Expresser 3-4, and PLD ζ2-Over Expresser 4-3 line. Values are means±SD (n=3). *P<0.05 difference from WT seeds by Student's t test.

FIG. 4 Shows the seed oil content and seed yield in PLDζ-OE camelina. (a) Oil content in seeds of three independent PLDζ2-Over Expresser (OE) camelina lines. Values are means±SD (n=5). *P<0.05 significant difference from WT seeds by Student's t test. (b) Staining for oil bodies in seeds by the dye nile red in WT and PLDζ2-OE (ζ2-1) seeds. Images were taken under confocal microscope using the same setting for both genotypes. Large oil bodies are shown by arrows. Bars=10 μm. (c) Total seed weight (g) per plants for WT and two independent PLDζ2-OE T2 camelina lines. Seeds were collected from plants grown in a greenhouse at the same time and under the same conditions as WT. Values are means±SD (n=12 for transgenic lines; n=6 for WT).

FIG. 5 Shows the overall growth performance and rate of seed germination of WT and PLDζ transgenic camelina. (a) Plant height and branch numbers. Data was collected from T3 matured plants (n=6 for WT; n=20 for each transgenic line). (b) Germination rate of WT and PLD transgenic camelina seeds. Values are mean±SD (n=150). For each transgenic line, 5 plants were used and 50 seeds from each plant were tested.

FIG. 6 Shows the oil, protein, and carbohydrate contents and seed yield in PLDζ-Over Expresser soybeans. (a) Oil content in different seed tissues in a transgenic line (J16) and the original cultivar Jack which was used for transformation. (b) Whole seed oil content of three independent PLDζ1-Over Expresser soybean lines and Jack. Inset, Immunoblotting of PLDζ1 in developing seeds (20 days post flowering). Proteins (20 μg/lane) were separated by SDS-PAGE followed by blotting with Flag antibodies. (c) Seed yields per plant relative to cultivar Jack. Seeds were collected from plants grown in a greenhouse at the same time as Jack. (d) Protein content in Jack and transgenic lines (n=10). (e) Cellulose, starch, and soluble sugar content of from 4 individual seeds. Values for all panels, excepted for those noted, are means±SD (n=5). *P<0.05 significant difference from WT seeds by Student's t test.

FIG. 7 Shows the growth performance in soybean cultivar Jack and PLDζ transgenic lines in T2 generation. (a) Plant height in matured plants. (b) The number of seed per plant. (c) Average weight per seed. For (a)-(c), Values are mean±SD (n=5 for WT, n=9 for transgenic lines. (d) Germination rate. Seeds were germinated for six days, and seeds which formed exposed radicle were recorded as germination. Values are mean±SD (n=20).

FIG. 8 Shows the oil content and seed yield in soybean cultivar Jack and PLD transgenic soybean in T3 generation. (a) Whole seed oil content in Jack and PLDζ1-Over Expresser lines. Values are means±SD (n=5). (b) Seed yield of Jack and seeds from T3 transgenic plants grown under the same condition. Values are means±SD (WT, n=5; J1A, n=12; J1B, n=5; J16, n=15)*P<0.05 difference from WT seeds by Student's t test.

FIG. 9 Shows a working model for the role of PLDs and PA in promoting TAG production in developing seeds. Increased expression of PLDζ increases the PC hydrolysis to produce PA that in turn increases PC synthesis by enhancing the expression of AAPTs. The enhanced production of PC and PA increases TAG production potentially via 1) more PA is converted to DAG that is incorporated to TAG by the Kennedy pathway, 2) PC is directly incorporated to TAG by PDAT, and 3) PC is converted by the reverse reaction of AAPT to DAG that is incorporated to TAG. AAPT, aminoalcoholphosphate transferase; CCT, choline-phosphate cytidylyltransferase; DGAT, diacylglycerol acyltransferase; DHAP, dihydroxyacetone-phosphate; Gly3P, glycerol-3-phosphate; GPAT, glycerol-3-phosphate acyltransferase; GPDH, glycerol 3 phosphate dehydrogenase; LPAAT, lysophosphatidic acid acyltransferase; PAP, PA phosphohydrolase; PDAT, PC: DAG acyltransferase.

FIG. 10 Shows PA and PC levels in developing seeds of PLDζ-altered Arabidopsis. (a) and (b), PA and PC contents in WT, pldζ1ζ2-KO, PLDζ1-Over Expresser 3-4, and PLD ζ2-Over Expresser 4-3 line during development. Siliques were used for 7 day-post-flowering samples whereas seeds were used for other stages. The last stage was mature seeds. Phospholipids were separated by TLC and quantified by GC analysis of fatty acid content. Values are means±SD (n=5). *P<0.05 difference from WT seeds by Student's t test.

FIG. 11 Shows the glycerolipid contents and PC and PA species in developing seeds of WT, PLD ζ1-Over Expresser 3-4, and PLD ζ2-Over Expresser 4-3 lines in Arabidopsis. (a) Polar glycerolipid composition in mol %. (b) Mol % of PC molecular species. (c) Mol % of PA molecular species. Developing seeds from 16 days post flowering were collected. Total lipids were extracted and profiled using tandem mass spectrometry. Numbers separated by colons refer to total acyl carbons:total acyl double bonds. Values are means±SD (n=5). *P<0.05 difference from WT seeds by Student's t test.

FIG. 12 Shows the expression of genes related to TAG biosynthesis in WT and PLDζ-altered Arabidopsis seeds. RNA was isolated from developing seeds (14 day post flowering) of WT (Col), pldζ1ζ2 double KO, PLD ζ1-Over Expresser 3-4, and PLD ζ2-Over Expresser 4-3 line. The transcript level of each gene was quantified by real-time PCR and expressed as relative to that of UBQ10. Values are means±SD (n=3). *P<0.05 difference from WT seeds by Student's t test. LPAAT, Lysophosphatidic acid acyltransferase; LPP, lipid phosphate phosphatase; DGAT, diacylglycerol acyltransferase; PDAT, PC: DAG acyltransferase; CCT, choline-phosphate cytidylyltransferase; AAPT, aminoalcoholphosphate transferase; ROD, reduced oleate desaturase; PAH, phosphatidic acid phosphohydrolase.

### DETAILED DESCRIPTION OF THE INVENTION

### Definitions

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The terms “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the terms “cell,” “cells,” “cell line,” “host cell,” and “host cells,” are used interchangeably and, encompass animal cells and include plant, invertebrate, non-mammalian vertebrate, insect, algal, and mammalian cells. All such designations include cell populations and progeny. Thus, the terms “transformants” and “transfectants” include the primary subject cell and cell lines derived therefrom without regard for the number of transfers.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

“Expression control sequences” are regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, that have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

As used herein, the term “fatty acids” refers to long chain aliphatic acids (alkanoic acids) of varying chain lengths, from about C₁₂ to C₂₂ (although both longer and shorter chain-length acids are known). The predominant chain lengths are between C₁₆ and C₂₂. The structure of a fatty acid is represented by a simple notation system of “X:Y”, where X is the total number of carbon (C) atoms in the particular fatty acid and Y is the number of double bonds. Additional details concerning the differentiation between “saturated fatty acids” versus “unsaturated fatty acids”, “monounsaturated fatty acids” versus “polyunsaturated fatty acids” (or “PUFAs”), and “omega-6 fatty acids” (ω-6 or n-6) versus “omega-3 fatty acids” (ω-3 or n-3) are provided in WO2004/101757. “PUFAs” can be classified into two major families (depending on the position (n) of the first double bond nearest the methyl end of the fatty acid carbon chain). Thus, the “ω-6 fatty acids” (ω-6 or n-6) have the first unsaturated double bond six carbon atoms from the omega (methyl) end of the molecule and additionally have a total of two or more double bonds, with each subsequent unsaturation occurring 3 additional carbon atoms toward the carboxyl end of the molecule. In contrast, the “co-3 fatty acids” (ω-3 or n-3) have the first unsaturated double bond three carbon atoms away from the omega end of the molecule and additionally have a total of three or more double bonds, with each subsequent unsaturation occurring 3 additional carbon atoms toward the carboxyl end of the molecule.

A “gene” is a sequence of nucleotides which code for a functional gene product. Generally, a gene product is a functional protein. However, a gene product can also be another type of molecule in a cell, such as RNA (e.g., a tRNA or an rRNA). A gene may also comprise expression control sequences (i.e., non-coding) sequences as well as coding sequences and introns. The transcribed region of the gene may also include untranslated regions including introns, a 5′-untranslated region (5′-UTR) and a 3′-untranslated region (3′-UTR).

The term “heterologous” refers to a nucleic acid or protein which has been introduced into an organism (such as a plant, animal, or prokaryotic cell), or a nucleic acid molecule (such as chromosome, vector, or nucleic acid construct), which are derived from another source, or which are from the same source, but are located in a different (i.e. non native) context.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention.

To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., (1987) Cell, 50:667). Such proteins (and their encoding nucleic acids) have sequence homology, as reflected by their sequence similarity, whether in terms of percent identity or by the presence of specific residues or motifs and conserved positions.

As used herein, the term “increase” or the related terms “increased”, “enhance” or “enhanced” refers to a statistically significant increase. For the avoidance of doubt, the terms generally refer to at least a 2% increase in a given parameter, and can encompass at least a 3% increase, 4% increase, 5% increase, 6% increase, 7% increase, 8% increase, 9% increase, 10% increase, 20% increase, 30% increase, 40% or even a 50% increase over the control value.

The term “isolated,” when used to describe a protein or nucleic acid, means that the material has been identified and separated and/or recovered from a component of its natural environment. Contaminant components of its natural environment are materials that would typically interfere with research, diagnostic or therapeutic uses for the protein or nucleic acid, and may include enzymes, hormones, and other proteinaceous or non-proteinaceous solutes. In some embodiments, the protein or nucleic acid will be purified to at least 95% homogeneity as assessed by SDS-PAGE under non-reducing or reducing conditions using Coomassie blue or, preferably, silver stain. Isolated protein includes protein in situ within recombinant cells, since at least one component of the protein of interest's natural environment will not be present. Ordinarily, however, isolated proteins and nucleic acids will be prepared by at least one purification step.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Molec. Biol. 215: 403-410 (1990) and Altschul et al. Nuc. Acids Res. 25: 3389-3402 (1997)).

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when the −27 cumulative alignment score falls off by the quantity X from its maximum achieved value, the cumulative score goes to zero or below due to the accumulation of one or more negative-scoring residue alignments, or the end of either sequence is reached. The BLAST algorithm parameters W. T. and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The term “oilseed plant” refers to plants that produce seeds or fruit with a high, i.e. have greater than about 10% oil content. Exemplary oil seed plants include for example, plants of the genus Camelina, coconut, cotton seed, peanut, rapeseed, safflower, sesame, soybean, sunflower, olive, corn and palm.

The terms “operably linked”, “operatively linked,” or “operatively coupled” and synonyms thereof are used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other embodiments, a nucleic acid molecule may additionally include one or more DNA or RNA nucleotide sequences chosen from: (a) a nucleotide sequence capable of increasing translation; (b) a nucleotide sequence capable of increasing secretion of the recombinant protein outside a cell; (c) a nucleotide sequence capable of increasing the mRNA stability, and (d) a nucleotide sequence capable of binding a trans-acting factor to modulate transcription or translation, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein; it is not necessarily contiguous with it. Operable linking of various nucleotide sequences is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

The terms “polynucleotide,” “nucleotide sequence” and “nucleic acid” are used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, a polynucleotide includes not only naturally occurring bases such as A, T, U, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example nopaline synthase (nos) promoter and octopine synthase (ocs) promoters carried on tumor-inducing plasmids of Agrobacterium tumefaciens and the caulimovirus promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), the Figwort Mosaic Virus (FMV) 35S promoter (U.S. Pat. No. 5,378,619), the cassaya vein mosaic virus (U.S. Pat. No. 7,601,885). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. Methods for purification are well-known in the art. As used herein, the term “substantially free” is used operationally, in the context of analytical testing of the material. Preferably, purified material substantially free of contaminants is at least 50% pure; more preferably, at least 75% pure, and more preferably still at least 95% pure. Purity can be evaluated by chromatography, gel electrophoresis, immunoassay, composition analysis, biological assay, and other methods known in the art. The term “substantially pure” indicates the highest degree of purity, which can be achieved using conventional purification techniques known in the art.

The term “sequence similarity” refers to the degree of identity or correspondence between nucleic acid or amino acid sequences that may or may not share a common evolutionary origin. However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

In particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

As used herein, the terms “triacylglycerol”, “TAGs” or “oil” refer to neutral lipids composed of three fatty acyl residues esterified to a glycerol molecule (and such terms will be used interchangeably throughout the present disclosure herein). Such oils can contain long chain poly unsaturated fatty acids, as well as shorter saturated and unsaturated fatty acids, longer chain saturated fatty acids and trace amounts of other lipophilic molecules including sterols, sterol esters, tocopherols, eicosanoids, glycoglycerolipids, glycosphingolipds, sphingolipids, and phospholipids. Thus, “oil biosynthesis” generically refers to the synthesis of TAGs in the cell. “Seed oils” are those oils naturally produced by plants during the development and maturation of seeds.

As used herein, a “transgenic plant” is one whose genome has been altered by the incorporation of heterologous genetic material, e.g. by transformation as described herein. The term “transgenic plant” is used to refer to the plant produced from an original transformation event, or progeny from later generations or crosses of a transgenic plant, so long as the progeny contains the heterologous genetic material in its genome.

The term “transformation” or “transfection” refers to the transfer of one or more nucleic acid molecules into a host cell or organism. Methods of introducing nucleic acid molecules into host cells include, for instance, calcium phosphate transfection, DEAE-dextran mediated transfection, microinjection, cationic lipid-mediated transfection, electroporation, scrape loading, ballistic introduction, or infection with viruses or other infectious agents.

“Transformed”, “transduced”, or “transgenic”, in the context of a cell, refers to a host cell or organism into which a recombinant or heterologous nucleic acid molecule (e.g., one or more DNA constructs or RNA, or siRNA counterparts) has been introduced. The nucleic acid molecule can be stably expressed (i.e. maintained in a functional form in the cell for longer than about three months) or non-stably maintained in a functional form in the cell for less than three months i.e. is transiently expressed. For example, “transformed,” “transformant,” and “transgenic” cells have been through the transformation process and contain foreign nucleic acid. The term “untransformed” refers to cells that have not been through the transformation process.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Buchanan et al., Biochemistry and Molecular Biology of Plants, Courier Companies, USA, 2000; Mild and Iyer, Plant Metabolism, 2^{nd} Ed. D. T. Dennis, D H Turpin, D D Lefebrve, D G Layzell (eds) Addison Wesly, Langgmans Ltd. London (1997); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### I. Overview

The present invention includes methods, DNA constructs, and transgenic plants that exhibit enhanced rates of oil production and improved oil content. In one aspect such methods and transgenic plants are created through the over expression of phospholipase D zeta. In certain embodiments the enzymes are expressed with seed tissues.

Accordingly, in one aspect the current invention includes a method for the production of a seed oil comprising the steps of: 1) transforming a plant cell with a nucleotide sequence encoding a PLDζ operatively linked to expression control sequences that drive expression of the PLDζ in the plant cell, 2) growing the transgenic plant, and 3) harvesting the seeds.

In another aspect, the current invention includes a method for increasing plant seed oil content comprising the steps of: i) providing a plant seed, and ii) overexpressing one or multiple enzymes of PLD zeta family in the seed under the control of a gene promoter that drives PLD expression in seeds

### II. Exemplary Phospholipase D Zeta Genes

In one embodiment, the phospholipase D zeta encodes an enzyme whose activity is substantially independent of calcium concentration, and which catalyzes the selective hydrolysis of phosphatidylcholine (PC) to produce phosphatic acid (PA). Such genes may be useful to selectively stimulate the production of PA within a developing seed, thereby up-regulating lipid synthesis.

In some embodiments, the enzyme is phospholipase D zeta 1 or zeta 2. In any of these methods, DNA constructs, and transgenic organisms, the terms “phospholipase D zeta”, or “PLD ζ” refers to all naturally-occurring and synthetic genes encoding a phospholipase D capable of selectively catalyzing the hydrolysis of PC to PA, in an essentially calcium independent fashion.

In one aspect the PLD ζ is from planta. In a further embodiment the PLD ζ is from Arabidopsis thaliana. Representative species and Gene bank accession numbers for various species of PLD ζ are listed below in Table D1, and genes from other species may be readily identified by standard homology searching of publicly available databases, based on the presence of the conserved HKD motif, and PX or PH domains common to all PLD ζs. (See generally Qin and Wang (2002) The Arabidopsis phospholipase D family. Characterization of a calcium-independent and phosphatidylcholine-selective PLD ζ with distinct regulatory domains. Plant Physiology 128 1057-1068).

TABLE D1
Exemplary phospholipase D zeta enzymes

| GenBank | | |
|---|---|---|
| Accession | | Seq. ID |
| Number and | | listing |
| organism | Sequence | No. |
| AAL06337.1 | MASEQLMSPA SGGGRYFQMQ PEQFPSMVSS LFSFAPAPTQ | SEQ. ID. |
| Arabidopsis | ETNRIFEELP KAVIVSVSRP DAGDISPVLL SYTIECQYKQ | No. 1 |
| thaliana | FKWQLVKKAS QVFYLHFALK KRAFIEEIHE KQEQVKEWLQ | |
| PLD zeta 1 | NLGIGDHPPV VQDEDADEVP LHQDESAKNR DVPSSAALPV | |
| | IRPLGRQQSI SVRGKHAMQE YLNHFLGNLD IVNSREVCRF | |
| | LEVSMLSFSP EYGPKLKEDY IMVKHLPKFS KSDDDSNRCC | |
| | GCCWFCCCND NWQKVWGVLK PGFLALLEDP FDAKLLDIIV | |
| | FDVLPVSNGN DGVDISLAVE LKDHNPLRHA FKVTSGNRSI | |
| | RIRAKNSAKV KDWVASINDA ALRPPEGWCH PHRFGSYAPP | |
| | RGLTDDGSQA QWFVDGGAAF AAIAAAIENA KSEIFICGWW | |
| | VCPELYLRRP FDPHTSSRLD NLLENKAKQG VQIYILIYKE | |
| | VALALKINSV YSKRRLLGIH ENVRVLRYPD HFSSGVYLWS | |
| | HHEKLVIVDN QVCFIGGLDL CFGRYDTFEH KVGDNPSVTW | |
| | PGKDYYNPRE SEPNTWEDAL KDELERKKHP RMPWHDVHCA | |
| | LWGPPCRDVA RHFVQRWNYA KRNKAPYEDS IPLLMPQHHM | |
| | VIPHYMGRQE ESDIESKKEE DSIRGIRRDD SFSSRSSLQD | |
| | IPLLLPHEPV DQDGSSGGHK ENGTNNRNGP FSFRKSKIEP | |
| | VDGDTPMRGF VDDRNGLDLP VAKRGSNAID SEWWETQDHD | |
| | YQVGSPDETG QVGPRTSCRC QIIRSVSQWS AGTSQVEESI | |
| | HSAYRSLIDK AEHFIYIENQ FFISGLSGDD TVKNRVLEAL | |
| | YKRILRAHNE KKIFRVVVVI PLLPGFQGGI DDSGAASVRA | |
| | IMHWQYRTIY RGHNSILTNL YNTIGVKAHD YISFYGLRAY | |
| | GKLSEDGPVA TSQVYVHSKI MIVDDRAALI GSANINDRSL | |
| | LGSRDSEIGV LIEDTELVDS RMAGKPWKAG KFSSSLRLSL | |
| | WSEHLGLRTG EIDQIIDPVS DSTYKEIWMA TAKTNTMIYQ | |
| | DVFSCVPNDL IHSRMAFRQS LSYWKEKLGH TTIDLGIAPE | |
| | KLESYHNGDI KRSDPMDRLK AIKGHLVSFP LDFMCKEDLR | |
| | PVFNESEYYA SPQVFH | |
| AAP68834.1 | MSTDKLLLPN GVKSDGVIRM TRADAAAAAA SSSLGGGSQI | SEQ. ID. |
| Arabidopsis | FDELPKAAIV SVSRPDTTDF SPLLLSYTLE LQYKQFKWTL | No. 2 |
| thaliana | QKKASQVLYL HFALKKRLII EELHDKQEQV REWLHSLGIF | |
| PLD zeta 2 | DMQGSVVQDD EEPDDGALPL HYTEDSIKNR NVPSRAALPI | |
| | IRPTIGRSET VVDRGRTAMQ GYLSLFLGNL DIVNSKEVCK | |
| | FLEVSRLSFA REYGSKMKEG YVTVKHLRDV PGSDGVRCCL | |
| | PTHCLGFFGT SWTKVWAVLK PGFLALLEDP FSGKLLDIMV | |
| | FDTLGLQGTK ESSEQPRLAE QVKEHNPLRF GFKVTSGDRT | |
| | VRLRTTSSRK VKEWVKAVDE AGCYSPHRFG SFAPPRGLTS | |
| | DGSQAQWFVD GHTAFEAIAF AIQNATSEIF MTGWWLCPEL | |
| | YLKRPFEDHP SLRLDALLET KAKQGVKIYI LLYKEVQIAL | |
| | KINSLYSKKR LQNIHKNVKV LRYPDHLSSG IYLWSHHEKI | |
| | VIVDYQVCFI GGLDLCFGRY DTAEHKIGDC PPYIWPGKDY | |
| | YNPRESEPNS WEETMKDELD RRKYPRMPWH DVHCALWGPP | |
| | CRDVARHFVQ RWNHSKRNKA PNEQTIPLLM PHHHMVLPHY | |
| | LGTREIDIIA AAKPEEDPDK PVVLARHDSF SSASPPQEIP | |
| | LLLPQETDAD FAGRGDLKLD SGARQDPGET SEESDLDEAV | |
| | NDWWWQIGKQ SDCRCQIIRS VSQWSAGTSQ PEDSIHRAYC | |
| | SLIQNAEHFI YIENQFFISG LEKEDTILNR VLEALYRRIL | |
| | KAHEENKCFR VVIVIPLLPG FQGGIDDFGA ATVRALMHWQ | |
| | YRTISREGTS ILDNLNALLG PKTQDYISFY GLRSYGRLFE | |
| | DGPIATSQIY VHSKLMIVDD RIAVIGSSNI NDRSLLGSRD | |
| | SEIGVVIEDK EFVESSMNGM KWMAGKFSYS LRCSLWSEHL | |
| | GLHAGEIQKI EDPIKDATYK DLWMATAKKN TDIYNQVFSC | |
| | IPNEHIRSRA ALRHNMALCK DKLGHTTIDL GIAPERLESC | |
| | GSDSWEILKE TRGNLVCFPL QFMCDQEDLR PGFNESEFYT | |
| | APQVFH | |
| XP_002883027.1 | MASEQLMSPA SGGGGRYFQM QPEQFPSMVS SLFSFAPAPT | SEQ. ID. |
| Arabidopsis | QESNRIFEEL PKAVIVSVSR PDAGDISPVL LSYTIECQYK | No. 3 |
| lyrata | QFKWQLVKKA SQVFYLHFAL KKRAFIEEIH EKQEQVKEWL | |
| subsp | QNLGIGDHAP VVQDEDADEV PLHQDESAKN RDVPSSAALP | |
| | VIRPLGRQQS ISVRGKHAMQ EYLNHFLGNL DIVNSREVCR | |
| | FLEVSMLSFS PEYGPKLKED YIMVKHLPKF SKSDDDSNRC | |
| | CGCCWFCCCN DNWQKVWGVL KPGFLALLED PFDAKLLDII | |
| | VFDVLPVSNG NDGVDVSLAV ELKDHNPLRH AFKVTSGNRS | |
| | IRIRAKSSAK VKDWVASIND AALRPPEGWC HPHRFGSYAP | |
| | PRGLTDDGSQ AQWFVDGGAA FAAIAAAIEN AKSEIFICGW | |
| | WVCPELYLRR PFDPHTSSRL DNLLENKAKQ GVQIYILLYK | |
| | EVALALKINS VYSKRRLLGI HENVRVLRYP DHFSSGVYLW | |
| | SHHEKLVIVD NQVCFIGGLD LCFGRYDTFE HKVGDNPSVT | |
| | WPGKDYYNPR ESEPNTWEDA LKDELNRKKH PRMPWHDVHC | |
| | ALWGPPCRDV ARHFVQRWNY AKRNKAPYED SIPLLMPQHH | |
| | MVIPHYMGRQ EESDTESKKD EDSIKGIRRD DSFSSRSSLQ | |
| | DIPLLLPQEP VDQDGSSRGH KENGTNNRNG PFSFRKLKIE | |
| | PVDGDTPMRG FVDDRNGLDL PVAKRGSNAI DSEWWETQEH | |
| | DYQVGSPDET GQVGPRTSCR CQIIRSVSQW SAGTSQVEES | |
| | IHSAYRSLID KAEHFIYIEN QFFISGLSGD DTIKNRILEA | |
| | LYKRILRAHN EKKSFRVVVV IPLLPGFQGG IDDSGAASVR | |
| | AIMHWQYRTI YRGHNSILTN LYNTIGAKAH DYISFYGLRA | |
| | YGKLSEDGPV ATSQVYVHSK IMIIDDRAAL IGSANINDRS | |
| | LLGSRDSEIG VLIEDTEFVD SRMAGKPWKA GKFSSSLRLS | |
| | LWSEHLGLRT GEIDQIIDPV SDSTYKEIWM ATAKTNTMIY | |
| | QDVFSCVPND LIHSRMAFRQ SLSYWKEKLG HTTIDLGIAP | |
| | EKLESYHNGD IKRSDPMDRL KSIKGHLVSF PLDFMCKEDL | |
| | RPVFNESEYY ASPQVFH | |
| XP_002272864.1 | MASEDLMSGA GARYIQMQSE PMPSTISSFF SFRQSPESTR | SEQ. ID. |
| Vitis vinifera | IFDELPKATI VFVSRPDASD ISPALLTYTI EFRYKQFKWR | No. 4 |
| | LIKKASQVFF LHFALKKRVI IEEIQEKQEQ VKEWLQNIGI | |
| | GEHTAVVHDD DEPDEETVPL HHDESVKNRD IPSSAALPII | |
| | RPALGRQNSV SDRAKVAMQG YLNLFLGNLD IVNSREVCKF | |
| | LEVSKLSFSP EYGPKLKEDY VMVKHLPKIP KEDDTRKCCP | |
| | CPWFSCCNDN WQKVWAVLKP GFLALLEDPF HPQPLDIIVF | |
| | DLLPASDGNG EGRLSLAKEI KERNPLRHAL KVTCGNRSIR | |
| | LRAKSSAKVK DWVAAINDAG LRPPEGWCHP HRFGSFAPPR | |
| | GLSEDGSLAQ WFVDGRAAFE AIASAIEEAK SEIFICGWWV | |
| | CPELYLRRPF HSHASSRLDA LLEAKAKQGV QIYILLYKEV | |
| | ALALKINSVY SKRKLLSIHE NVRVLRYPDH FSTGVYLWSH | |
| | HEKLVIVDYQ ICFIGGLDLC FGRYDTLEHK VGDHPPLMWP | |
| | GKDYYNPRES EPNSWEDTMK DELDRGKYPR MPWHDVHCAL | |
| | WGPPCRDVAR HFVQRWNYAK RNKAPNEQAI PLLMPQQHMV | |
| | IPHYMGRSRE MEVEKKNVEN NYKDIKKLDS FSSRSSFQDI | |
| | PLLLPQEPDG LDSPHGESKL NGRSLSFSFR KSKIEPVPDM | |
| | PMKGFVDDLD TLDLKGKMSS DIMAQPGMRT CDREWWETQE | |
| | RGNQVLSADE TGQVGPCVPC RCQVIRSVSQ WSAGTSQVED | |
| | STHNAYCSLI EKAEHFIYIE NQFFISGLSG DEIIRNRVLE | |
| | VLYRRIMQAY NDKKCFRVII VIPLLPGFQG GLDDGGAASV | |
| | RAIMHWQYRT ICRGNNSILQ NLYDVIGHKT HDYISFYGLR | |
| | AYGRLFDGGP VASSQVYVHS KIMIVDDCTT LIGSANINDR | |
| | SLLGSRDSEI GVLIEDKELV DSYMGGKPKK AGKFAHSLRL | |
| | SLWSEHLGLR GGEIDQIKDP VVDSTYRDVW MATAKTNSTI | |
| | YQDVFSCIPN DLIHSRAAMR QHMAIWKEKL GHTTIDLGIA | |
| | PMKLESYDNG DMKTIEPMER LESVKGHLVY FPLDFMCKED | |
| | LRPVFNESEY YASPQVFH | |
| XP_002516974.1 | MASSEQLMNG SNGPRYVQMQ SEPSTPQHNQ QQLQQQHPSS | SEQ. ID. |
| Ricinus | MLSSFFSFTH GVTPESTRIF DELPTATIVS VSRPDAGDIS | No. 5 |
| communis | PVLLTYTIEF KWQLSKKAAQ VFYLHFALKR RAFFEEIHEK | |
| | QEQVKEWLQN LGIGDHTPVV QDDDDADDET ILLHNEESAK | |
| | NRNVPSRAAL PVIRPALGRQ HSMSDRAKVA MQEYLNHFLG | |
| | NLDIVNSREV CKFLEVSKLS FSLEYGPKLK EDYVMARHLP | |
| | PIPTNDDSGK CCACHWFSCC NDNWQKVWAV LKPGFLALLA | |
| | DPFDAKPLDI IVFDVLPASD GSGEGRISLA METKERNPLR | |
| | HAFKVTCGVR SIKLRTKTGA RVKDWVAAIN DAGLRPPEGW | |
| | CHPHRFGSFA PPRGLTEDGS QAQWFIDGMA AFDAIASSIE | |
| | DAKSEIFICG WWLCPELYLR RPFHAHASSR LDDLLEAKAK | |
| | QGVQIYILLY KEVALALKIN SVYSKRKLLS IHENVRVLRY | |
| | PDHFSSGVYL WSHHEKLVIV DYQICFIGGL DLCFGRYDTR | |
| | EHRVGDCPPF VWPGKDYYNP RESEPNSWED TMKDELDRKK | |
| | YPRMPWHDVH CALWGPPCRD VARHFVQRWN YAKRNKAPYE | |
| | EAIPLLMPQH HMVIPHYRGS SKDLEVETKN GEDDSKGIKR | |
| | EDSFSSRSSL QDIPLLLPQE AEGTDGSGRG PKLNGLDSTP | |
| | GRSRSYAFRK SKFEAVVPDT PMKGFVDDHN ILDLHVKISP | |
| | DILPQSGTKT SHLEWWETQE RGDQVGFGDE TGQVGPRTSC | |
| | RCQVIRSVSQ WSAGTSQVEE SIHCAYRSLI EKAEHFIYIE | |
| | NQFFISGLSG DEIIRNRVLE SLYRRIMRAH NEKKCFRVII | |
| | VIPLIPGFQG GLDDSGAASV RAIMHWQYRT ICRGQNSIFH | |
| | NLYDVLGPKT HDYISFYGLR AYGKLFDGGP VATSQVYVHS | |
| | KIMIIDDCAT LIGSANINDR SLLGSRDSEI AVLIEDKEMV | |
| | DSFMGGRHWK AGKFSLSLRL SLWSEHLGLN AKEMKQIIDP | |
| | VIDSTYKDIW IATAKTNTTI YQDVFSCIPN DLMHSRAALR | |
| | QNMAFWKERL GHTTIDLGIA PEKLESYENG DIKKHDPMER | |
| | LQAVRGHLVS FPLDFMCRED LRPVFNESEY YASQVFY | |
| XP_002328619.1 | MQSEPSTPLQ PPSSSIISSF FSFRQGSTPE SGRIFDELPQ | SEQ. ID. |
| Populus | ATIVSVSRPD PSDISPVQLS YTIEVQYKQF KWRLLKKAAQ | No. 6 |
| trichocarpa | VFYLHFALKK RVFFEEILEK QEQVKEWLQN LGIGDHTPMV | |
| | NDDDDADDET IPLHHDESAK NRDVPSSAAL PVIRPALGRQ | |
| | NSMSDRAKVT MQQYLNHFLG NMDIVNSREV CKFLEVSKLS | |
| | FSPEYGPKLK EEYVMVKHLP RIVKDDDSRK CCACSWFSCC | |
| | NDNWQKVWAV LKPGFLALLA DPFDTKLLDI IVFDVLPASD | |
| | GSGEGRVSLA AEIKERNPLR HGFKVACGNR SIDLRSKNGA | |
| | RVKDWVATIN DAGLRPPEGW CHPHRFASFA PPRGLSEDGS | |
| | QAQWFVDGRA AFEAIALSIE DAKSEIFICG WWLCPELYLR | |
| | RPFRAHASSR LDSLLEAKAK QGVQIYILLY KEVALALKIN | |
| | SVYSKTKLLS IHENVRVLRY PDHFSTGVYL WSHHEKLVIV | |
| | DHQICFIGGL DLCFGRYDTC EHRVGDCPPQ VWPGKDYYNP | |
| | RESEPNSWED MMKDELDRGK YPRMPWHDVH CALWGPPCRD | |
| | VARHFVQRWN YAKRSKAPYE EAIPLLMPQQ HMVIPHYMGQ | |
| | NREMEVERKG IKDDVKGIKR QDSFSSRSSL QDIPLLLPQE | |
| | AEGPDDSGVG PKLNGMDSTP GRSLPHAFWK SKIELVVPDI | |
| | SMTSFVDNNG SDLHVKMSSD FSAQPGTKAS DLEWWETQER | |
| | VDQVGSPDES GQVGPRVSCH CQVIRSVSQW SAGTSQIEES | |
| | IHCAYCSLIE KAEHFVYIEN QFLISGLSGD DIIRNRVLEA | |
| | LYRRIMRAFN DKKCFRVIIV IPLLPGFQGG VDDGGAASVR | |
| | AIMHWQYRTI CRGQNSILHN LYDHLGPKTH DYISFYGLRS | |
| | YGRLFDGGPV ATSQVYVHSK IMIIDDRTTL IGSANINDRS | |
| | LLGSRDSEIG VLIEDKELVD SLMGGKPRKA GKFTLSLRLS | |
| | LWSEHLGLHS KAINKVIDPV IDSTYKDIWM STAKTNTMIY | |
| | QDVFSCVPND LIHTRAALRQ SMVSRKDRLG HTTIDLGIAP | |
| | QKLESYQNGD IKNTDPLERL QSTRGHLVSF PLEFMCKEDL | |
| | RPVFNESEYY ASQVFH | |

It is well established that the genetic code is degenerate and that some amino acids have multiple codons, and accordingly, multiple polynucleotides can encode the PLD ζ of the invention. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

In general, non-native nucleic acids that encode PLD ζ proteins can be obtained from by “back-translation” (for example by using Computer programs such as “BackTranslate” (GCG™ Package, Acclerys, Inc. San Diego, Calif.) of the deduced coding sequences derived from PLD ζ genomic clones, from cDNA or EST sequences, or any of the sequences listed in Table D1.

Examples of nucleic acids that contain mature PLD ζ protein-encoding nucleotide sequences include but are not limited to a sequence with at least 70%, at least 80%, at least 85%, at least 90%, at least 95%, at least 97%, at least 99%, or 100% sequence identity to SEQ. ID. No. 7, or SEQ. ID. No, 8 as listed in Table D2 below.

TABLE D2
Exemplary PLD zeta nucleic acids

| Clone | | SEQ. ID. |
|---|---|---|
| Name | Sequence | No. |
| PLD ζ | ATGGCATCTG AGCAGTTGAT GTCTCCCGCC AGTGGTGGTG GACGCTACTT | SEQ. ID. |
| 1 | TCAGATGCAG CCTGAGCAAT TTCCTTCGAT GGTCTCTTCG CTCTTCTCTT | No. 7 |
| | TCGCGCCGGC TCCTACGCAG GAGACTAATC GTATTTTTGA AGAATTACCA | |
| | AAAGCAGTGA TCGTCTCTGT CTCTCGCCCT GATGCCGGCG ATATTAGCCC | |
| | TGTACTCTTG TCTTACACCA TTGAGTGCCA ATACAAGCAG TTCAAGTGGC | |
| | AGCTTGTGAA GAAAGCATCT CAAGTCTTTT ATTTGCATTT TGCATTGAAG | |
| | AAACGTGCTT TTATTGAAGA AATTCACGAG AAGCAGGAAC AGGTTAAAGA | |
| | ATGGCTTCAA AATCTAGGAA TAGGGGATCA TCCACCCGTT GTGCAAGATG | |
| | AAGATGCTGA TGAAGTTCCG CTACATCAAG ATGAAAGTGC CAAAAATAGA | |
| | GATGTTCCTT CGAGCGCTGC TTTGCCAGTC ATTCGTCCTT TGGGAAGACA | |
| | GCAGTCCATA TCAGTTAGAG GAAAGCATGC AATGCAAGAA TATCTGAATC | |
| | ATTTTCTGGG GAATCTTGAT ATCGTCAATT CACGGGAGGT TTGCAGGTTT | |
| | TTGGAGGTTT CGATGTTGTC ATTCTCACCA GAGTATGGGC CCAAATTGAA | |
| | AGAAGACTAT ATCATGGTAA AACATCTACC GAAGTTTTCA AAGAGTGATG | |
| | ATGATTCTAA TAGATGTTGT GGGTGCTGTT GGTTCTGTTG CTGCAACGAT | |
| | AATTGGCAAA AGGTGTGGGG GGTACTAAAG CCAGGTTTTC TCGCCTTATT | |
| | GGAAGATCCA TTTGATGCGA AGCTATTAGA TATAATTGTT TTTGATGTCC | |
| | TACCAGTTTC TAATGGAAAT GATGGTGTGG ATATATCACT AGCAGTAGAA | |
| | CTGAAGGATC ATAATCCTTT GCGGCATGCA TTCAAGGTAA CATCTGGAAA | |
| | CCGGAGTATA AGAATAAGGG CAAAGAATAG TGCAAAAGTT AAAGATTGGG | |
| | TGGCTTCTAT TAACGATGCT GCTCTTAGAC CTCCTGAGGG TTGGTGCCAT | |
| | CCCCATCGCT TTGGCTCATA TGCTCCGCCG AGGGGTTTGA CGGATGACGG | |
| | AAGTCAAGCC CAGTGGTTTG TAGATGGTGG AGCAGCTTTT GCAGCCATTG | |
| | CTGCAGCGAT TGAAAATGCT AAATCTGAGA TTTTCATCTG TGGCTGGTGG | |
| | GTGTGCCCAG AACTCTATCT TAGGCGTCCT TTTGACCCGC ATACTTCATC | |
| | CAGACTTGAT AACTTGTTGG AGAATAAAGC TAAGCAAGGA GTTCAGATAT | |
| | ACATCCTTAT CTACAAGGAG GTTGCTCTTG CTTTAAAGAT CAACAGTGTA | |
| | TATAGCAAAC GCAGGCTTCT TGGCATTCAT GAGAATGTGC GGGTACTTCG | |
| | TTATCCTGAT CATTTCTCAA GTGGTGTCTA CCTCTGGTCT CACCATGAAA | |
| | AACTCGTCAT CGTCGATAAT CAGGTTTGCT TTATCGGAGG GCTAGACTTG | |
| | TGTTTTGGCC GATATGACAC GTTTGAACAT AAAGTTGGAG ATAACCCTTC | |
| | TGTGACATGG CCTGGAAAGG ACTATTACAA CCCCAGAGAG TCTGAACCCA | |
| | ATACTTGGGA GGATGCTCTG AAAGATGAAT TAGAGCGTAA AAAGCATCCA | |
| | CGGATGCCTT GGCATGATGT GCATTGTGCT TTATGGGGAC CACCTTGCCG | |
| | TGATGTGGCT AGGCACTTTG TTCAACGCTG GAACTATGCT AAGAGAAACA | |
| | AAGCACCATA TGAGGATTCA ATTCCGCTTC TTATGCCTCA ACATCACATG | |
| | GTTATACCCC ACTACATGGG AAGGCAAGAG GAGTCAGACA TTGAAAGCAA | |
| | GAAAGAGGAA GACAGTATTA GAGGGATTAG AAGAGATGAT TCATTTTCTT | |
| | CTAGATCATC TTTGCAGGAC ATTCCATTAC TTTTGCCTCA CGAACCAGTT | |
| | GATCAGGATG GTTCGAGTGG GGGGCATAAA GAAAATGGAA CAAACAACAG | |
| | AAATGGTCCT TTCTCTTTCC GGAAATCAAA AATTGAACCA GTTGATGGAG | |
| | ATACTCCTAT GAGGGGCTTT GTAGATGATC GTAATGGGCT AGATCTTCCA | |
| | GTAGCAAAGC GTGGTTCTAA TGCAATAGAT TCAGAGTGGT GGGAAACACA | |
| | AGATCATGAT TATCAGGTTG GGTCGCCAGA TGAGACTGGG CAAGTCGGTC | |
| | CGAGAACTTC ATGCCGCTGT CAGATTATAC GAAGTGTCAG TCAGTGGTCT | |
| | GCCGGTACAA GCCAAGTTGA AGAGAGTATC CATTCTGCTT ACCGTTCTCT | |
| | CATTGACAAA GCTGAACATT TTATCTACAT TGAGAATCAG TTTTTCATAT | |
| | CAGGCCTTTC TGGAGATGAC ACAGTAAAGA ACCGTGTCTT AGAAGCATTG | |
| | TACAAGAGGA TTTTGCGTGC CCATAACGAG AAGAAAATTT TCAGGGTTGT | |
| | TGTTGTTATA CCTCTCCTCC CCGGTTTCCA GGGAGGTATT GACGACAGTG | |
| | GTGCAGCATC TGTTAGAGCC ATAATGCATT GGCAGTATCG AACCATATAC | |
| | AGAGGACATA ACTCAATATT GACTAATCTT TACAATACTA TTGGCGTAAA | |
| | GGCTCATGAT TATATTTCCT TCTATGGCCT TAGGGCATAT GGTAAACTTT | |
| | CTGAGGATGG ACCTGTCGCC ACTAGTCAGG TGTATGTTCA CAGTAAAATC | |
| | ATGATAGTTG ATGACCGTGC TGCATTGATT GGATCTGCCA ATATTAACGA | |
| | CCGGAGTTTG CTTGGCTCAA GAGATTCTGA GATTGGAGTA CTAATCGAAG | |
| | ACACAGAGTT AGTAGATTCT CGCATGGCAG GAAAACCATG GAAGGCTGGA | |
| | AAATTTTCTT CAAGTCTTAG GCTCTCTTTG TGGTCCGAAC ACCTTGGACT | |
| | TCGTACTGGA GAGATCGACC AGATTATTGA TCCCGTCTCT GATTCAACCT | |
| | ACAAGGAGAT ATGGATGGCA ACCGCAAAGA CAAACACAAT GATATACCAG | |
| | GATGTCTTCT CTTGTGTGCC CAATGATCTC ATCCATTCAA GAATGGCCTT | |
| | CAGACAAAGC CTATCGTATT GGAAAGAGAA GCTGGGACAC ACAACGATCG | |
| | ATTTGGGAAT AGCACCAGAG AAGCTGGAGT CTTACCACAA TGGAGACATC | |
| | AAGAGAAGCG ATCCAATGGA CAGACTAAAG GCGATAAAAG GACATCTCGT | |
| | CTCTTTCCCT TTAGATTTCA TGTGCAAAGA AGATCTAAGA CCGGTCTTCA | |
| | ATGAGAGTGA ATACTACGCC TCCCCTCAAG TCTTCCATTG A | |
| PLD ζ | ATGTCGACGG ATAAATTACT ACTTCCTAAC GGCGTTAAGT CAGACGGAGT | SEQ. ID. |
| 2 | CATCAGAATG ACCAGAGCTG ATGCTGCGGC GGCGGCAGCT TCTTCTTCTC | No. 8 |
| | TCGGCGGTGG AAGTCAAATA TTCGACGAGC TTCCCAAGGC TGCGATCGTC | |
| | TCGGTCTCGA GACCTGACAC CACCGATTTT AGTCCCTTGC TTCTTTCTTA | |
| | CACCTTGGAG CTTCAGTATA AACAGTTCAA GTGGACATTA CAAAAGAAGG | |
| | CTTCTCAAGT TCTGTACTTA CATTTTGCGT TGAAGAAACG TTTGATCATT | |
| | GAAGAACTTC ACGACAAGCA AGAACAGGTT AGAGAGTGGC TACACAGCTT | |
| | GGGGATTTTT GATATGCAAG GATCAGTTGT GCAAGATGAT GAAGAACCTG | |
| | ACGATGGTGC TCTTCCTCTG CACTATACTG AAGATAGTAT CAAGAACAGG | |
| | AATGTTCCTT CCCGTGCAGC GCTTCCAATC ATTCGTCCAA CGATAGGCCG | |
| | GTCAGAGACA GTTGTAGATC GTGGGAGAAC CGCAATGCAA GGCTACTTGA | |
| | GTCTCTTTCT AGGGAACTTG GACATTGTAA ACTCCAAAGA GGTCTGCAAG | |
| | TTCCTAGAAG TTTCTAGACT CTCATTTGCT AGAGAGTACG GTTCCAAGAT | |
| | GAAAGAAGGG TATGTCACAG TGAAGCACTT GAGGGACGTC CCAGGTTCTG | |
| | ATGGTGTCCG ATGCTGTCTT CCTACACACT GTCTCGGTTT CTTCGGAACT | |
| | AGCTGGACAA AGGTTTGGGC GGTTCTGAAA CCAGGATTTT TGGCGTTACT | |
| | AGAAGATCCA TTCAGCGGAA AGCTTCTAGA TATAATGGTG TTCGACACAT | |
| | TGGGGTTGCA AGGTACTAAA GAGTCTTCTG AACAACCGCG TTTGGCTGAA | |
| | CAGGTGAAGG AACACAACCC ATTGCGTTTT GGCTTTAAAG TTACTAGTGG | |
| | GGACCGAACC GTGAGGCTGA GAACAACGAG CAGCAGGAAA GTTAAAGAGT | |
| | GGGTTAAGGC CGTGGACGAA GCTGGTTGTT ACAGTCCACA TCGGTTTGGT | |
| | TCGTTTGCAC CACCTAGAGG CTTGACATCG GACGGAAGCC AGGCACAGTG | |
| | GTTCGTAGAC GGTCACACTG CGTTTGAAGC TATCGCGTTT GCAATCCAAA | |
| | ACGCAACATC AGAGATATTT ATGACTGGTT GGTGGTTATG TCCGGAGCTA | |
| | TATCTCAAAC GCCCCTTTGA AGATCATCCA TCATTGCGGC TCGATGCATT | |
| | GCTGGAGACA AAAGCAAAAC AGGGCGTTAA GATATATATT CTTCTGTATA | |
| | AGGAAGTCCA AATCGCGCTG AAAATCAACA GCTTGTACAG CAAGAAACGG | |
| | CTTCAAAACA TTCACAAGAA CGTCAAAGTT CTTCGTTATC CAGACCATCT | |
| | CTCCTCCGGC ATTTACCTCT GGTCGCACCA CGAGAAAATA GTGATTGTAG | |
| | ATTACCAAGT TTGTTTCATT GGAGGGTTAG ATCTCTGTTT TGGGCGGTAC | |
| | GATACAGCGG AGCACAAGAT TGGAGATTGC CCTCCTTATA TATGGCCTGG | |
| | AAAAGATTAC TACAATCCTA GAGAATCTGA ACCAAATTCG TGGGAAGAAA | |
| | CGATGAAAGA TGAGTTAGAC AGGAGAAAGT ACCCGCGAAT GCCGTGGCAC | |
| | GATGTCCACT GCGCTCTATG GGGACCGCCT TGTCGGGATG TGGCTCGACA | |
| | TTTTGTCCAG CGGTGGAACC ACTCTAAGAG AAACAAGGCA CCTAATGAAC | |
| | AGACGATTCC ATTGTTGATG CCTCACCACC ACATGGTTCT TCCTCACTAC | |
| | TTGGGAACTA GAGAGATCGA TATAATCGCA GCGGCTAAAC CAGAGGAAGA | |
| | CCCTGACAAA CCTGTCGTTC TTGCCAGACA TGACTCTTTC TCTTCTGCCT | |
| | CACCGCCCCA AGAAATCCCT TTGCTTCTCC CACAAGAAAC CGATGCAGAT | |
| | TTCGCCGGCA GAGGAGATCT GAAGTTAGAC AGCGGTGCAA GACAAGATCC | |
| | TGGGGAAACT TCAGAGGAAA GCGATCTGGA CGAGGCTGTG AACGACTGGT | |
| | GGTGGCAGAT TGGGAAGCAG AGTGATTGCC GGTGTCAAAT AATCAGAAGT | |
| | GTTAGCCAAT GGTCTGCTGG GACGAGCCAG CCTGAAGATA GCATTCATAG | |
| | AGCTTATTGT TCGCTTATCC AGAACGCTGA ACATTTTATC TACATAGAGA | |
| | ACCAATTCTT CATCTCCGGG CTAGAAAAAG AGGACACGAT CCTAAACCGC | |
| | GTTCTAGAAG CGTTATACAG ACGCATTCTG AAGGCTCATG AAGAGAACAA | |
| | GTGCTTCCGC GTTGTGATCG TTATTCCGCT ACTCCCTGGA TTTCAGGGAG | |
| | GTATTGATGA CTTCGGAGCA GCCACGGTTC GAGCACTGAT GCATTGGCAA | |
| | TACCGTACGA TCTCTAGAGA AGGAACTTCG ATTCTTGACA ACCTTAACGC | |
| | TTTGCTCGGT CCCAAGACGC AAGATTACAT CTCTTTCTAT GGTTTGAGAT | |
| | CGTACGGACG GCTGTTTGAG GACGGTCCAA TTGCCACTAG CCAGATTTAC | |
| | GTGCATAGCA AGTTAATGAT TGTTGATGAC CGGATCGCAG TGATCGGATC | |
| | TTCTAATATA AACGATAGGA GCTTACTAGG TTCACGAGAC TCTGAGATCG | |
| | GTGTTGTGAT TGAAGACAAA GAATTCGTGG AATCTTCGAT GAACGGAATG | |
| | AAGTGGATGG CCGGGAAGTT CTCTTACAGT CTTAGATGTT CCTTGTGGTC | |
| | AGAGCATCTC GGCCTTCACG CCGGAGAGAT TCAGAAGATC GAAGATCCAA | |
| | TCAAAGATGC AACATACAAA GACTTATGGA TGGCAACAGC TAAGAAAAAC | |
| | ACGGACATCT ACAACCAAGT CTTCTCGTGC ATCCCGAATG AACATATACG | |
| | CTCAAGAGCT GCATTGAGAC ACAATATGGC TCTTTGTAAA GACAAGTTGG | |
| | GTCACACTAC GATCGACCTT GGCATTGCAC CGGAGAGGCT AGAATCATGC | |
| | GGCAGCGACT CGTGGGAGAT TCTGAAGGAG ACAAGAGGGA ACCTTGTGTG | |
| | CTTCCCATTA CAGTTCATGT GTGATCAAGA AGATCTCAGA CCAGGTTTCA | |
| | ACGAATCTGA GTTCTACACT GCTCCTCAAG TCTTCCACTA A | |

In some embodiments, the non-native PLD ζ-encoding nucleotide sequence can designed so that it will be highly expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the non-native PLD ζ-encoding nucleotide sequence are codons that are more abundant in monocot and/or dicot plant genes. Codon usage in various monocot or dicot genes have been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the non-native PLD ζ-encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the PLD ζ polynucleotide sequences listed in Table D2. Polynucleotide “variants” may contain one or more substitutions, additions, deletions and/or insertions in relation to a reference polynucleotide. Generally, variants of the PLD ζ reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence (i.e. to any of the sequences in Table D2) or their corresponding genomic clones (SEQ. ID. No. 9, and SEQ. ID. No. 10) as determined by sequence alignment programs described elsewhere herein using default parameters.

In some embodiments the PLD ζ which may be used in any of the methods and plants of the invention may have amino acid sequences which are substantially homologous, or substantially similar to any of the native PLD ζ amino acid sequences, for example, to any of the native PLD ζ amino acid sequences encoded by the genes listed in Table D1.

For use in the present invention, the PLD ζ may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of PLD ζ, are also specifically included in any of the methods of the invention including for example, pyroglutamyl, iso-aspartyl, proteolytic, phosphorylated, glycosylated, reduced, oxidatized, isomerized, and deaminated variants of the PLD ζ.

Alternatively, the PLD ζ may have an amino acid sequence having at least 30% preferably at least 40, 50, 60, 70, 75, 80, 85, 90, 95, 98, or 99% identity with a PLD ζ encoded by a gene listed in Table D1. In a preferred embodiment, the PLD ζ for use in any of the methods and plants of the present invention is at least 80% identical to the mature PLD ζ 1 or PLD ζ 2 from Arabidopsis thaliana (SEQ. ID. NO. 1 or 2).

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations can be introduced into PLD ζ and are considered within the scope of the invention. Mutations of PLD ζ that increase the activity of the protein are known and may be used in the methods and plants of the invention. The PLD ζ may thus include one or more amino acid deletions, additions, insertions, and/or substitutions based on any of the naturally-occurring isoforms of PLD ζ. These may be contiguous or non-contiguous. Representative variants may include those having 1 to 8, or more preferably 1 to 4, 1 to 3, or 1 or 2 amino acid substitutions, insertions, and/or deletions as compared to any of sequences listed in Table D1.

The variants, derivatives, and fusion proteins of PLD ζ are functionally equivalent in that they have detectable PLD ζ activity. More particularly, they exhibit at least 5%, at least 10%, at least 20%, at least 30%, at least 40%, preferably at least 60%, more preferably at least 80% of the activity of PLD ζ 1 or 2 from Arabidopsis thaliana, and are thus they are capable of substituting for PLD ζ from Arabidopsis.

All such variants, derivatives, fusion proteins, or fragments of PLD ζ are included, and may be used in any of the polynucleotides, vectors, host cell and methods disclosed and/or claimed herein, and are subsumed under the term “PLD ζ”. Suitable assays for determining functional PLD ζ activity are well known in the art, and are described for example in Qin and Wang (2002) Plant Physiology 128 1057-1068).

### III. DNA Constructs

In some embodiments, the DNA constructs, and expression vectors of the invention include expression vectors comprising a nucleic acid encoding a PLD ζ operatively coupled to a promoter, and transcriptional terminator for efficient expression in the organism of interest. In one aspect of any of these expression vectors, the PLD ζ is codon optimized for expression in the organism of interest. In one aspect of any of these expression vectors, the PLD ζ is operatively coupled to a seed specific promoter. In some embodiments, the nucleic acid encoding the PLD ζ-encodes an amino acid sequence which is at least 80% identical to a PLD ζ from Table D1. In some embodiments, the nucleic acid encoding the PLD ζ is at least 80% identical to a DNA sequence listed in Table D2.

In some embodiments, the PLD ζ DNA constructs and expression vectors of the invention further comprise polynucleotide sequences encoding one or more of the following elements i) a selectable marker gene to enable antibiotic selection, ii) a screenable marker gene to enable visual identification of transformed cells, and iii) T-element DNA sequences to enable Agrobacterium tumefaciens mediated transformation. Exemplary expression cassettes are described in the Examples.

Those of skill in the art will appreciate that the foregoing descriptions of expression cassettes represents only illustrative examples of expression cassettes that could be readily constructed, and is not intended to represent an exhaustive list of all possible DNA constructs or expression cassettes that could be constructed.

Moreover expression vectors suitable for use in expressing the claimed DNA constructs in plants, and methods for their construction are generally well known, and need not be limited. These techniques, including techniques for nucleic acid manipulation of genes such as subcloning a subject promoter, or nucleic acid sequences encoding a gene of interest into expression vectors, labeling probes, DNA hybridization, and the like, and are described generally in Sambrook, et al., Molecular Cloning—A Laboratory Manual (2nd Ed.), Vol. 1-3, Cold Spring Harbor Laboratory, Cold Spring Harbor, N.Y., 1989, which is incorporated herein by reference. For instance, various procedures, such as PCR, or site directed mutagenesis can be used to introduce a restriction site at the start codon of a heterologous gene of interest. Heterologous DNA sequences are then linked to a suitable expression control sequences such that the expression of the gene of interest are regulated (operatively coupled) by the promoter.

DNA constructs comprising an expression cassette for the gene of interest can then be inserted into a variety of expression vectors. Such vectors include expression vectors that are useful in the transformation of plant cells. Many other such vectors useful in the transformation of plant cells can be constructed by the use of recombinant DNA techniques well known to those of skill in the art as described above.

Exemplary expression vectors for expression in protoplasts or plant tissues include pUC 18/19 or pUC 118/119 (GIBCO BRL, Inc., MD); pBluescript SK (+/−) and pBluescript KS (+/−) (STRATAGENE, La Jolla, Calif.); pT7Blue T-vector (NOVAGEN, Inc., WI); pGEM-3Z/4Z (PROMEGA Inc., Madison, Wis.), and the like vectors, such as is described herein.

Exemplary vectors for expression using Agrobacterium tumefaciens-mediated plant transformation include for example, pBin 19 (CLONETECH), Frisch et al, Plant Mol. Biol., 27:405-409, 1995; pCAMBIA 1200 and pCAMBIA 1201 (Center for the Application of Molecular Biology to International Agriculture, Can berra, Australia); pGA482, An et al, EMBO J., 4:277-284, 1985; pCGN1547, (CALGENE Inc.) McBride et al, Plant Mol. Biol., 14:269-276, 1990, and the like vectors, such as is described herein.

Expression Control Sequences:

DNA constructs will typically include expression control sequences comprising promoters to drive expression of the PLD ζ within the photosynthetic organism. Promoters may provide ubiquitous, cell type specific, constitutive promoter or inducible promoter expression. Basal promoters in plants typically comprise canonical regions associated with the initiation of transcription, such as CAAT and TATA boxes. The TATA box element is usually located approximately 20 to 35 nucleotides upstream of the initiation site of transcription. The CAAT box element is usually located approximately 40 to 200 nucleotides upstream of the start site of transcription. The location of these basal promoter elements result in the synthesis of an RNA transcript comprising nucleotides upstream of the translational ATG start site. The region of RNA upstream of the ATG is commonly referred to as a 5′ untranslated region or 5′ UTR. It is possible to use standard molecular biology techniques to make combinations of basal promoters, that is, regions comprising sequences from the CAAT box to the translational start site, with other upstream promoter elements to enhance or otherwise alter promoter activity or specificity.

In some aspects promoters may be altered to contain “enhancer DNA” to assist in elevating gene expression. As is known in the art certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are particularly useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (U.S. Pat. No. 5,593,874), the maize shrunken 1 gene, the light sensitive 1 gene of Solanum tuberosum, and the heat shock protein 70 gene of Petunia hybrida (U.S. Pat. No. 5,659,122).

Depending upon the host cell system utilized, any one of a number of suitable promoters can be used. Promoter selection can be based on expression profile and expression level. The following are representative non-limiting examples of promoters that can be used in the expression cassettes.

Constitutive Expression:

Constitutive promoters typically provide for the constant and substantially uniform production of proteins in all tissues. Exemplary constitutive promoters include for example, the core promoter of the Rsyn7 (U.S. patent application Ser. No. 08/661,601), the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. patent application Ser. No. 08/409,297), and the like. Other constitutive promoters include, for example those disclosed in, U.S. Pat. Nos. 5,608,149; 5,608,144; 5,604,121; 5,569,597; 5,466,785; 5,399,680; 5,268,463; and 5,608,142.

Tissue Specific Expression:

Tissue-specific promoters include those described in Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc. Natl. Acad. Sci. U.S.A. 90(20):9586-9590; and Guevara-Garcia et al. (1993) Plant J. 4(3):495-505. Root specific promoters include, for example, those disclosed in Hire, et al (1992) Plant Mol. Biology, 20(2): 207-218; Keller and Baumgartner, (1991) The Plant Cell, 3(10): 1051-1061; Sanger et al. (1990) Plant Mol. Biology, 14(3): 433-443; Miao et al. (1991) The Plant Cell, 3(1): 11-22; Bogusz et al. (1990) The Plant Cell, 2(7): 633-641. Seed-preferred promoters includes both seed-specific promoters (those promoters active during seed development) as well as seed-germinating promoters (those promoters active during seed germination). Such promoters include beta conglycinin, (Fujiwara & Beachy (1994) Plant. Mol. Biol. 24 261-272); Ciml (cytokinin-induced message); cZ19B1 (maize 19 KDa zein); mi1ps (myo-inositol-1-phosphate synthase); celA (cellulose synthase); end1 (Hordeum verlgase mRNA clone END1); and imp3 (myo-inositol monophosphate-3). For dicots, particular promoters include phaseolin, napin, β-conglycinin, soybean lectin, and the like. For monocots, particular promoters include maize 15 Kd zein, 22 KD zein, 27 kD zein, waxy, shrunken 1, shrunken 2, globulin 1, etc. In certain embodiments the DNA constructs, transgenic plants and methods use the oleosin promoter and/or napin promoter.

Inducible Expression:

Chemically Inducible Promoters. A chemically induced promoter element can be used to replace, or in combination with any of the foregoing promoters to enable the chemically inducible expression of the PLD ζ throughout a plant, or within a specific tissue. For example the expression of trans factor comprising the ecdysone receptor operatively coupled to a GAL4 DNA binding domain and VP16 activation domain can be used to regulate the expression of a second gene that is operatively coupled to a minimal promoter and GAL4 (5×UAS sequences) in a ligand depend fashion. A number of useful EcRs are known in the art, and have been used to develop ligand regulated gene switches. Specific examples of EcR based gene switches include for example those disclosed in U.S. Pat. No. 6,723,531, U.S. Pat. No. 5,514,578, U.S. Pat. No. 6,245,531, U.S. Pat. No. 6,504,082, U.S. Pat. No. 7,151,168, U.S. Pat. No. 7,205,455, U.S. Pat. No. 7,238,859, U.S. Pat. No. 7,456,315, U.S. Pat. No. 7,563,928, U.S. Pat. No. 7,091,038, U.S. Pat. No. 7,531,326, U.S. Pat. No. 7,776,587, U.S. Pat. No. 7,807,417, U.S. Pat. No. 7,601,508, U.S. Pat. No. 7,829,676, U.S. Pat. No. 7,919,269, U.S. Pat. No. 7,563,879, U.S. Pat. No. 7,297,781, U.S. Pat. No. 7,312,322, U.S. Pat. No. 6,379,945, U.S. Pat. No. 6,610,828, U.S. Pat. No. 7,183,061 and U.S. Pat. No. 7,935,510. In addition, other chemical regulators can also be employed to induce expression of the selected coding sequence in the plants transformed according to the presently disclosed subject matter, including the benzothiadiazole, isonicotinic acid, salicylic acid, for example as disclosed in U.S. Pat. Nos. 5,523,311, 5,614,395, and 5,880,333 herein incorporated by reference.

The promoter of choice is preferably excised from its source by restriction enzymes, but can alternatively be PCR-amplified using primers that carry appropriate terminal restriction sites.

The selected target gene coding sequence can be inserted into this vector, and the fusion products (i.e., promoter-gene-terminator) can subsequently be transferred to any selected transformation vector, including those described below.

Transcriptional Terminators:

A variety of transcriptional terminators are available for use in the DNA constructs of the invention. These are responsible for the termination of transcription beyond the transgene and its correct polyadenylation.

Appropriate transcriptional terminators are those that are known to function in the relevant plant system. Representative plant transcriptional terminators include the CaMV 35S terminator, the tml terminator, the nopaline synthase terminator (NOS ter), and the pea rbcS E9 terminator. In certain embodiments, the inventions utilize the oleosin terminator and/or napin terminator. With regard to RNA polymerase III terminators, these terminators typically comprise a −52 run of 5 or more consecutive thymidine residues. In one embodiment, an RNA polymerase III terminator comprises the sequence TTTTTTT. These can be used in both monocotyledons and dicotyledons.

Sequences for the Enhancement or Regulation of Expression:

Numerous sequences have been found to enhance the expression of an operatively lined nucleic acid sequence, and these sequences can be used in conjunction with the nucleic acids of the presently disclosed subject matter to increase their expression in transgenic plants.

Various intron sequences have been shown to enhance expression, particularly in monocotyledonous cells. For example, the introns of the maize Adbl gene have been found to significantly enhance the expression of the wild-type gene under its cognate promoter when introduced into maize cells. Intron 1 was found to be particularly effective and enhanced expression in fusion constructs with the chloramphenicol acetyltransferase gene. In the same experimental system, the intron from the maize bronzes gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMY) have been shown to be effective in enhancing expression.

Selectable Markers:

For certain target species, different antibiotic or herbicide selection markers can be included in the methods, DNA constructs, and transgenic organisms of the invention. Selection markers used routinely in transformation include the npt II gene (Kan), which confers resistance to kanamycin and related antibiotics, the bar gene, which confers resistance to the herbicide phosphinothricin, the hph gene, which confers resistance to the antibiotic hygromycin, the dhfr gene, which confers resistance to methotrexate, and the EPSP synthase gene, which confers resistance to glyphosate (U.S. Pat. Nos. 4,940,935 and 5,188,642).

Screenable Markers:

Screenable markers may also be employed in the methods, DNA constructs and transgenic organisms of the present invention, including for example the β-glucuronidase or uidA gene (the protein product is commonly referred to as GUS), isolated from E. coli, which encodes an enzyme for which various chromogenic substrates are known; an R-locus gene, which encodes a product that regulates the production of anthocyanin pigments (red color) in plant tissues; a β-lactamase gene, which encodes an enzyme for which various chromogenic substrates are known (e.g., PADAC, a chromogenic cephalosporin); a xylE gene, which encodes a catechol dioxygenase that can convert chromogenic catechols; an α-amylase gene; a tyrosinase gene which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene, which allows for bioluminescence detection; an aequorin gene, which may be employed in calcium-sensitive bioluminescence detection; or a gene encoding for green fluorescent protein (PCT Publication WO 97/41228).

The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which has the genotype r-g, b, P1. Alternatively, any genotype of maize can be utilized if the C1 and R alleles are introduced together.

In some aspects, screenable markers provide for visible light emission as a screenable phenotype. A screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for population screening for bioluminescence, such as on tissue culture plates, or even for whole plant screening. The gene which encodes green fluorescent protein (GFP) is contemplated as a particularly useful reporter gene (PCT Publication WO 97/41228). Expression of green fluorescent protein may be visualized in a cell or plant as fluorescence following illumination by particular wavelengths of light. Where use of a screenable marker gene such as lux or GFP is desired, the inventors contemplated that benefit may be realized by creating a gene fusion between the screenable marker gene and a selectable marker gene, for example, a GFP—NPTII gene fusion (PCT Publication WO 99/60129). This could allow, for example, selection of transformed cells followed by screening of transgenic plants or seeds. In a similar manner, it is possible to utilize other readily available fluorescent proteins such as red fluorescent protein (CLONTECH, Palo Alto, Calif.).

Transformation: Techniques for transforming a wide variety of plant species are well known and described in the technical and scientific literature. See, for example, Weising et al, (1988) Ann. Rev. Genet., 22:421-477. As described herein, the DNA constructs of the present invention typically contain a marker gene which confers a selectable phenotype on the plant cells. For example, the marker may encode biocide resistance, particularly antibiotic resistance, such as resistance to kanamycin, G418, bleomycin, hygromycin, or herbicide resistance, such as resistance to chlorsulfuron or Basta. Such selective marker genes are useful in protocols for the production of transgenic plants.

DNA constructs can be introduced into the genome of the desired plant host by a variety of conventional techniques. For example, the DNA construct may be introduced directly into the plant cell using techniques such as electroporation and microinjection of plant cell protoplasts. Alternatively, the DNA constructs can be introduced directly to plant tissue using biolistic methods, such as DNA micro-particle bombardment. In addition, the DNA constructs may be combined with suitable transfer DNA (T-DNA) flanking regions and introduced into a conventional Agrobacterium tumefaciens Ti Plasmid. The T-DNA of the Ti plasmid will be transferred into plant cell through Agrobacterium-mediated transformation system.

Microinjection techniques are known in the art and well described in the scientific and patent literature. The introduction of DNA constructs using polyethylene glycol precipitation is described in Paszkowski et al, (1984) EMBO J., 3:2717-2722. Electroporation techniques are described in Fromm et al, (1985) Proc. Natl. Acad. Sci. USA, 82:5824. Biolistic transformation techniques are described in Klein et al, (1987) Nature 327:70-7. The full disclosures of all references cited are incorporated herein by reference.

A variation involves high velocity biolistic penetration by small particles with the nucleic acid either within the matrix of small beads or particles, or on the surface (Klein et al., (1987), Nature, 327:70-73). Although typically only a single introduction of a new nucleic acid segment is required, this method particularly provides for multiple introductions.

Agrobacterium tumefaciens-meditated transformation techniques are well described in the scientific literature. See, for example Horsch et al, (1984) Science, 233:496-498, and Fraley et al, (1983) Proc. Natl. Acad. Sci. USA, 90:4803.

More specifically, a plant cell, an explant, a meristem or a seed is infected with Agrobacterium tumefaciens transformed with the segment. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., (1984), Science, 233:496-498; Fraley et al., (1983), Proc. Nat'l. Acad. Sci. U.S.A., 80:4803).

Ti plasmids contain two regions essential for the production of transformed cells. One of these, named transfer DNA (T DNA), induces tumor formation. The other, termed virulent region, is essential for the introduction of the T DNA into plants. The transfer DNA region, which transfers to the plant genome, can be increased in size by the insertion of the foreign nucleic acid sequence without its transferring ability being affected. By removing the tumor-causing genes so that they no longer interfere, the modified Ti plasmid can then be used as a vector for the transfer of the gene constructs of the invention into an appropriate plant cell, such being a “disabled Ti vector”.

All plant cells which can be transformed by Agrobacterium and whole plants regenerated from the transformed cells can also be transformed according to the invention so as to produce transformed whole plants which contain the transferred foreign nucleic acid sequence. There are various ways to transform plant cells with Agrobacterium, including: (1) co-cultivation of Agrobacterium with cultured isolated protoplasts, (2) co-cultivation of cells or tissues with Agrobacterium, or (3) transformation of developing embryos, leaves, apices, or meristems with Agrobacterium.

Method (1) requires an established culture system that allows culturing protoplasts and plant regeneration from cultured protoplasts. Method (2) requires (a) that the plant cells or tissues can be transformed by Agrobacterium and (b) that the transformed cells or tissues can be induced to regenerate into whole plants. Method (3) requires micropropagation.

In the binary system, to have infection, two plasmids are needed: a T-DNA containing plasmid and a vir plasmid. Any one of a number of T-DNA containing plasmids can be used, the only requirement is that one be able to select independently for each of the two plasmids. After transformation of the plant cell or plant, those plant cells or plants transformed by the Ti plasmid so that the desired DNA segment is integrated can be selected by an appropriate phenotypic marker. These phenotypic markers include, but are not limited to, antibiotic resistance, herbicide resistance or visual observation. Other phenotypic markers are known in the art and may be used in this invention.

The present invention embraces use of the claimed modified PLD ζ constructs in transformation of any plant, including both dicots and monocots. Transformation of dicots is described in references above. Transformation of monocots is known using various techniques including electroporation (e.g., Shimamoto et al., (1992), Nature, 338:274-276); ballistics (e.g., European Patent Application 270,356); and Agrobacterium (e.g., Bytebier et al., (1987), Proc. Nat'l Acad. Sci. USA, 84:5345-5349).

Transformed plant cells which are derived by any of the above transformation techniques can be cultured to regenerate a whole plant which possesses the desired transformed phenotype. Such regeneration techniques rely on manipulation of certain phytohormones in a tissue culture growth medium typically relying on a biocide and/or herbicide marker which has been introduced together with the nucleotide sequences. Plant regeneration from cultured protoplasts is described in Evans et al, Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally by Klee et al, (1987) Ann Rev. Plant Phys., 38:467-486. Additional methods for producing a transgenic plant useful in the present invention are described in U.S. Pat. Nos. 5,188,642; 5,202,422; 5,384,253; 5,463,175; and 5,639,947. The methods, compositions, and expression vectors of the invention have use over a broad range of types of plants, including the creation of transgenic plant species belonging to virtually any species including for example, canola, camelina, flax, alfalfa, soybean, cotton, corn, rice, wheat, barley and etc.

Selection:

Typically DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin, G418 and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase (hpt).

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

One example of an herbicide which is useful for selection of transformed cell lines in the practice of the invention is the broad spectrum herbicide glyphosate. Glyphosate inhibits the action of the enzyme EPSPS, which is active in the aromatic amino acid biosynthetic pathway Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, PCT Publication WO 97/04103. The best characterized mutant EPSPS gene conferring glyphosate resistance comprises amino acid changes at residues 102 and 106, although it is anticipated that other mutations will also be useful (PCT Publication WO 97/04103). Furthermore, a naturally occurring glyphosate resistant EPSPS may be used, e.g., the CP4 gene isolated from Agrobacterium encodes a glyphosate resistant EPSPS (U.S. Pat. No. 5,627,061).

To use the bar-bialaphos or the EPSPS-glyphosate selective systems, tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is believed that ranges of 0.1-50 mg/l bialaphos or 0.1-50 mM glyphosate will find utility in the practice of the invention. Bialaphos and glyphosate are provided as examples of agents suitable for selection of transformants, but the technique of this invention is not limited to them.

Another herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism. Synthetic PPT, the active ingredient in the herbicide LIBERTY™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity. The bar gene has been cloned and expressed in transgenic tobacco, tomato, potato, Brassica and maize (U.S. Pat. No. 5,550,318). In previous reports, some transgenic plants which expressed the resistance gene were completely resistant to commercial formulations of PPT and bialaphos in greenhouses.

It further is contemplated that the herbicide dalapon, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (U.S. Pat. No. 5,780,708).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468 and U.S. Pat. No. 6,118,047.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells that are expressing luciferase and manipulate cells expressing in real time. Another screenable marker which may be used in a similar fashion is the gene coding for green fluorescent protein (GFP) or a gene coding for other fluorescing proteins such as DSRED® (Clontech, Palo Alto, Calif.).

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase or GFP would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene (WO 99/60129).

Regeneration and Seed Production:

Cells that survive the exposure to the selective agent, or cells that have been scored positive in a screening assay, may be cultured in media that supports regeneration of plants. In an exemplary embodiment, MS and N6 media may be modified by including further substances such as growth regulators. Preferred growth regulators for plant regeneration include cytokins such as 6-benzylamino pierine, zeahin or the like, and abscisic acid. Media improvement in these and like ways has been found to facilitate the growth of cells at specific developmental stages. Tissue may be maintained on a basic media with auxin type growth regulators until sufficient tissue is available to begin plant regeneration efforts, or following repeated rounds of manual selection, until the morphology of the tissue is suitable for regeneration, then transferred to media conducive to maturation of embryoids. Cultures are transferred every 1-4 weeks, preferably every 2-3 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets were transferred to soiless plant growth mix, and hardened off, e.g., in an environmentally controlled chamber at about 85% relative humidity, 600 ppm CO₂, and 25-250 microeinsteins m⁻²s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous expressible gene. Note however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

Characterization:

To confirm the presence of the exogenous DNA or “transgene(s)” in the regenerating plants, a variety of assays, known in the art may be performed. Such assays include, for example, “molecular biological” assays, such as Southern and Northern blotting and PCR; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

DNA Integration, RNA Expression and Inheritance:

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR). Using this technique discreet fragments of DNA are amplified and detected by gel electrophoresis. This type of analysis permits one to determine whether a gene is present in a stable transformant, but does not necessarily prove integration of the introduced gene into the host cell genome. Typically, DNA has been integrated into the genome of all transformants that demonstrate the presence of the gene through PCR analysis. In addition, it is not possible using PCR techniques to determine whether transformants have exogenous genes introduced into different sites in the genome, i.e., whether transformants are of independent origin. Using PCR techniques it is possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition, it is possible through Southern hybridization to demonstrate the presence of introduced genes in high molecular weight DNA, i.e., confirm that the introduced gene has been integrated into the host cell genome. The technique of Southern hybridization provides information that is obtained using PCR, e.g., the presence of a gene, but also demonstrates integration into the genome and characterizes each individual transformant.

It is contemplated that using the techniques of dot or slot blot hybridization, which are modifications of Southern hybridization techniques, one could obtain the same information that is derived from PCR, e.g., the presence of a gene.

Both PCR and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR techniques, referred to as RT-PCR, also may be used for detection and quantification of RNA produced from introduced genes. In this application of PCR it is first necessary to reverse transcribe RNA into DNA, using enzymes such as reverse transcriptase, and then through the use of conventional PCR techniques amplify the DNA. In most instances PC techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

It is further contemplated that TAQMAN® technology (Applied Biosystems, Foster City, Calif.) may be used to quantitate both DNA and RNA in a transgenic cell.

Gene Expression:

While Southern blotting and PCR may be used to detect the gene(s) in question, they do not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following an increase in fluorescence as anthranilate is produced, to name two.

Very frequently the expression of a gene product is determined by evaluating the phenotypic results of its expression. These assays also may take many forms, including but not limited to, analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

Event Specific Transgene Assay:

Southern blotting, PCR and RT-PCR techniques can be used to identify the presence or absence of a given transgene but, depending upon experimental design, may not specifically and uniquely identify identical or related transgene constructs located at different insertion points within the recipient genome. To more precisely characterize the presence of transgenic material in a transformed plant, one skilled in the art could identify the point of insertion of the transgene and, using the sequence of the recipient genome flanking the transgene, develop an assay that specifically and uniquely identifies a particular insertion event. Many methods can be used to determine the point of insertion such as, but not limited to, GENOME WALKER™ technology (CLONTECH, Palo Alto, Calif.), VECTORETTE™ technology (Sigma, St. Louis, Mo.), restriction site oligonucleotide PCR, uneven PCR (Chen and Wu, (1997), Gene, 185: 195-1199) and generation of genomic DNA clones containing the transgene of interest in a vector such as, but not limited to, lambda phage.

Once the sequence of the genomic DNA directly adjacent to the transgenic insert on either or both sides has been determined, one skilled in the art can develop an assay to specifically and uniquely identify the insertion event. For example, two oligonucleotide primers can be designed, one wholly contained within the transgene and one wholly contained within the flanking sequence, which can be used together with the PCR technique to generate a PCR product unique to the inserted transgene. In one embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the transgene. In another embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the genomic sequence adjacent to the insertion site. Confirmation of the PCR reaction may be monitored by, but not limited to, size analysis on gel electrophoresis, sequence analysis, hybridization of the PCR product to a specific radiolabeled DNA or RNA probe or to a molecular beacon, or use of the primers in conjugation with a TAQMAN™ probe and technology (Applied Biosystems, Foster City, Calif.).

Site Specific Integration or Excision of DNA Sequences:

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome and multiple copies of a construct may integrate. This random insertion of introduced DNA into the genome of host cells can be detrimental to the cell if the foreign DNA inserts into an essential gene. In addition, the expression of a transgene may be influenced by “position effects” caused by the surrounding genomic DNA. Further, because of difficulties associated with plants possessing multiple transgene copies, including gene silencing, recombination and unpredictable inheritance, it is typically desirable to control the copy number of the inserted DNA, often only desiring the insertion of a single copy of the DNA sequence. Furthermore, site-specific integration or excision offers a means to create a mutated gene of interest by adding or deleting sequences as designed for example to modify the expression of a native PLD ζ gene in a plant species of interest.

Site-specific integration can be achieved in plants by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a host plant cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus only those DNA sequences located between the two regions sharing genomic homology become integrated into the genome.

Although introduced sequences can be targeted for insertion into a specific genomic site via homologous recombination, in higher eukaryotes homologous recombination is a relatively rare event compared to random insertion events. Thus random integration of transgenes is more common in plants. To maintain control over the copy number and the location of the inserted DNA, randomly inserted DNA sequences can be removed. One manner of removing these random insertions is to utilize a site-specific recombinase system (U.S. Pat. No. 5,527,695).

A recently invented synthetic zinc finger nuclease (ZFNs) technology provides a powerful tool to modify the genome of given species by adding or deleting DNA sequences. ZFNs function as dimers with each monomer composed of a synthetic zinc finger domain fused with a nonspecific cleavage domain of the Fold endonuclease. The zinc finger domain in each of the monomers recognizes and binds to specific sequences in the genome as designed, typically 18 or 24 by depending on the number of zinc fingers in the synthetic zinc finger domain. Two ZFN monomer recognition sites are spaced by 5 to 7 bp. The zinc finger domain in the ZFN monomers will direct the Fold to the two adjacent DNA recognition sites of the ZFN monomers, form a functional Fold dimer and generate a DNA double-strand break (DSB) in the spacer sequence between the two zinc finger recognition sites (Zhang et al., (2010), Proc. Nat'l Acad. Sci. USA 107:12028-1203; Cui et al. (2011), Nature Biotechnology 29: 64-68). During the process of repairing chromosome breaks, nonhomologous end-joining or homologous recombination will occur which will greatly enhance the frequencies of targeted integration or deletion of DNA sequences. This method has been demonstrated very effective in Arabidopsis (Zhang et al., (2010) PNAS 107:12028-1203) and can be employed to create mutants of an endogenous PLD gene in a plant species of interest, for example to increase its expression in seeds.

A number of different site specific recombinase systems could be employed in accordance with the instant invention, including, but not limited to, the Cre/lox system of bacteriophage P1 (U.S. Pat. No. 5,658,772, specifically incorporated herein by reference in its entirety), the FLP/FRT system of yeast, the Gin recombinase of phage Mu, the Pin recombinase of E. coli, and the R/RS system of the pSR1 plasmid. The bacteriophage P1 Cre/10× and the yeast FLP/FRT systems constitute two particularly useful systems for site specific integration or excision of transgenes. In these systems, a recombinase (Cre or FLP) will interact specifically with its respective site-specific recombination sequence (10× or FRT, respectively) to invert or excise the intervening sequences. The sequence for each of these two systems is relatively short (34 by for 10× and 47 by for FRT) and therefore, convenient for use with transformation vectors.

The FLP/FRT recombinase system has been demonstrated to function efficiently in plant cells. Experiments on the performance of the FLP/FRT system in both maize and rice protoplasts indicate that FRT site structure, and amount of the FLP protein present, affects excision activity. In general, short incomplete FRT sites leads to higher accumulation of excision products than the complete full-length FRT sites. The systems can catalyze both intra- and intermolecular reactions in maize protoplasts, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between lox sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, (1987), Mol. Cell. Biol. 7:2087-2096). Cre was expressed from an inducible yeast GAL1 promoter and this Cre gene was located on an autonomously replicating yeast vector.

Since the lox site is an asymmetrical nucleotide sequence, lox sites on the same DNA molecule can have the same or opposite orientation with respect to each other. Recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

Deletion of Sequences Located within the Transgenic Insert:

During the transformation process it is often necessary to include ancillary sequences, such as selectable marker or reporter genes, for tracking the presence or absence of a desired trait gene transformed into the plant on the DNA construct. Such ancillary sequences often do not contribute to the desired trait or characteristic conferred by the phenotypic trait gene. Homologous recombination is a method by which introduced sequences may be selectively deleted in transgenic plants.

It is known that homologous recombination results in genetic rearrangements of transgenes in plants. Repeated DNA sequences have been shown to lead to deletion of a flanked sequence in various dicot species, e.g. Arabidopsis thaliana and Nicotiana tabacum. One of the most widely held models for homologous recombination is the double-strand break repair (DSBR) model.

Deletion of sequences by homologous recombination relies upon directly repeated DNA sequences positioned about the region to be excised in which the repeated DNA sequences direct excision utilizing native cellular recombination mechanisms. The first fertile transgenic plants are crossed to produce either hybrid or inbred progeny plants, and from those progeny plants, one or more second fertile transgenic plants are selected which contain a second DNA sequence that has been altered by recombination, preferably resulting in the deletion of the ancillary sequence. The first fertile plant can be either hemizygous or homozygous for the DNA sequence containing the directly repeated DNA which will drive the recombination event.

The directly repeated sequences are located 5′ and 3′ to the target sequence in the transgene. As a result of the recombination event, the transgene target sequence may be deleted, amplified or otherwise modified within the plant genome. In the preferred embodiment, a deletion of the target sequence flanked by the directly repeated sequence will result.

Alternatively, directly repeated DNA sequence mediated alterations of transgene insertions may be produced in somatic cells. Preferably, recombination occurs in a cultured cell, e.g., callus, and may be selected based on deletion of a negative selectable marker gene, e.g., the periA gene isolated from Burkholderia caryolphilli which encodes a phosphonate ester hydrolase enzyme that catalyzes the hydrolysis of glyceryl glyphosate to the toxic compound glyphosate (U.S. Pat. No. 5,254,801).

Iv. Trans Genic Organisms

In one aspect the invention also contemplates a transgenic organism comprising: a nucleic acid sequence comprising a polynucleotide sequence encoding a heterologous PLD ζ gene, or portion thereof; wherein the heterologous PLD ζ gene is over expressed in the transgenic organism compared to the wild type organism. In some embodiments, the heterologous PLD ζ gene is operatively coupled to a seed specific promoter.

The transgenic organisms therefore can contain one or more DNA constructs as defined herein as a part of the organism, the DNA constructs having been introduced by transformation of the organism.

In one aspect such transgenic organisms are characterized by having a seed oil content which is at least about 2% higher, at least about 3% higher, at least about 4% higher, at least about 5% higher, at least about 6% higher, at least about 8% higher, or at least about 10% higher than corresponding wild type organism.

In another aspect such transgenic organisms are characterized by having an increase in the relative levels (mol %) of linoleic (18:2), linolenic (18:3), and gondoic (20:1) fatty acids when compared to the fatty acid composition of WT seed oils.

In another aspect such transgenic organisms are characterized by having a decrease in palmitic (16:0), stearic (18:0), and oleic (18:1) when compared to the fatty acid composition of WT seed oils.

In some embodiments of these transgenic organisms the protein content of the seeds are approximately the same (i.e. within about 10 to about 20%) to the protein content of wild type seeds. In some embodiments of these transgenic organisms the carbohydrate content of the seeds are decreased by about 2% to about 5%.

In any of these transgenic characteristics, it will be understood that the transgenic organism will be grown using standard growth conditions as disclosed in the Examples, and compared to the equivalent wild type species.

In some embodiments the transgenic organism is from planta. In some embodiments the transgenic plant is an oilseed plant. In some embodiments the transgenic plant is from the family Brassicaceae. In some embodiments the transgenic plant is from the genus Camelina. In different aspects, the transgenic plant is selected from Camelina alyssum, Camelina microcarpa, Camelina runelica and Camelina sativa.

In some embodiments the transgenic plant is from the bean family Fabaceae. In some embodiments the transgenic plant is from the genus Glycine. In different aspects, the transgenic plant is selected from Glycine albicans, Glycine aphyonota, Glycine arenaria, Glycine argyrea, Glycine canescens, Glycine clandestine, Glycine curvata, Glycine cyrtoloba, Glycine falcate, Glycine gracei, Glycine hirticaulis, Glycine hirticaulis subsp. Leptosa, Glycine lactovirens, Glycine latifolia, Glycine latrobeana, Glycine microphylla, Glycine montis-douglas, Glycine peratosa, Glycine pescadrensis, Glycine pindanica, Glycine pullenii, Glycine rubiginosa, Glycine stenophita, Glycine syndetika, Glycine tabacina, Glycine tomentella, Glycine soja and Glycine max.

In certain embodiments of the transgenic plants, the PLD ζ gene is expressed primarily in the seed tissue of the transgenic plant. In this context, the term “primarily” means that the relative expression of the PLD ζ is at least about 150%, or at least about 200%, or at least about 300%, or at least about 400%, or at least about 500% higher in the seed tissue (on a dry weight by dry weight basis) compared to any other plant tissue, in the mature full developed plant, when grown under standard growth conditions.

### EXAMPLES

### General Experimental Methods

Plant Materials and Genetic Manipulation.

Isolation and verification of homozygous single and double mutant plants from the two Arabidopsis thaliana ecotypes, Columbia-0 (Col) and Wassilewskija (WS), are previously described. To overexpress PLDζ1 and PLDζ2, the full-length cDNA-coding region was amplified using the PLD cDNAs cloned from Col-1 and primers (forward 5′-CGGGCGGCCGCGGAAGACTTGAGGGGAGGCG (SEQ. ID. No. 11) and reverse 5′-CGGGCGGCCGCAGAGAAATGGCATCTGAGCA (SEQ. ID. No. 12) for PLDζ1; forward 5′-CGGGCGGCCGCAGTGGAAGACTTGAGGAGCA (SEQ. ID. No. 13) and reverse 5′-CGGGCGGCCGCGACGACGGTTTGGGGAGTTA (SEQ. ID. No. 14) for PLDζ2). The PCR product was cloned into BetaConSoyhyg vector that contains the seed-specific β-conglycinin promoter. The β-conglycinin promoter plus coding region was amplified using the forward primer, 5′-CGGGGTACCCGCGCCAAGCTTTTGATCCA (SEQ. ID. No. 15) for both PLDζs, and reverse primer 5′-CGCGGATCCGGAAGACTTGAGGGGAGGCG (SEQ. ID. No. 16) for PLDζ1 and 5′-CGCGGATCCGGTGGAAGACTTGAGGAGCA (SEQ. ID. No. 17) for PLD. The products were cloned into the binary vector p35S-FAST which has a C-terminal flag/strep tag fusion. The vector was transformed into the Agrobacterium strain GV3101 by a freezing and thawing method and then introduced into wild-type Arabidopsis Col-0 for overexpression via floral dipping. Transgenic plants were selected on 50 mg/L kanamycin. For mutant isolation and routine plant growth, seeds were sown in soil and treated at 4° C. for 2 days to break dormancy. Plants were grown in growth chambers under 16-hr-day/8-hr-night and 21° C./18° C. cycle.

The same PLDζ constructs were introduced into Camelina sativa using vacuum infiltration as described²⁷. The transgenic plants were selected on media containing 1% sucrose, 1×MS salts (pH adjusted to 5.7 using KOH), 0.28% Phytoblend (Caisson Laboratories), 50 mg/L kanamycin, and 150 mg/L carbenicillin. The putative transgenic seedlings were transferred to soil and leaves were collected for confirmation of the presence of PLDζ transgene by PCR. Developing seeds were then collected for immunoblotting for the presence of the introduced PLDζ protein. Camelina was grown in greenhouse at 21° C. with approximately 14 hr light.

For constructing the PLDζ vector for soybean transformation, the β-conglycinin promoter plus coding region and terminator was amplified using the forward primer, 5′-GCGGGCGCGCCCGCGCCAAGCTTTTGATCCAT (SEQ. ID. No. 18), and reverse primer 5′-ATGGCGCGCCAGTCACGACGTTGTA (SEQ. ID. No. 19) for both PLDζs. The cassette was digested with Asc I and ligated to the binary vector pZY101-ASCI which harbored a gene for Basta resistance. The resulting vectors were transformed into the Agrobacterium strain EHA101 by a freeze-thaw method. Soybean (cv. Jack) was subjected to Agrobacterium-mediated cotyledonary node transformation and transformants were selected by resistance to the herbicide glufosinate²⁸. Soybean was cultivated in greenhouse with supplemental lighting with 16-hr-day/8-hr-night and 30° C./18° C. cycle.

PLDζ immunoblotting and activity assays. Total proteins were extracted from developing siliques of Arabidopsis or camelina using buffer A (50 mM Tri-HCl, pH 7.5, 10 mM KCl, 1 mM EDTA, 2 mM DTT, and 0.5 mM phenylmethylsulfonyl fluoride). After centrifugation at 3000 g for 10 min, proteins in the supernatant were separated by 8% SDS-PAGE and transferred onto a polyvinylidene difluoride membrane. The membrane was blotted with anti-flag antibody (1:2000) overnight, followed by incubation with a second antibody (1:5000) conjugated with horseradish peroxidase for 1 hr. The membrane was washed with TBS/T four times and then incubated in LumiGLO substrate for 1 min, followed by exposure to X-ray film.

Total protein from developing seeds (20 days post-flowering) was extracted by grinding in an ice-chilled mortar and pestle with buffer A. The homogenate was centrifuged at 1000 g for 10 min, and the resulting supernatant was centrifuged at 100,000 g for 60 min The microsomal, pellet fraction was used for PLDζ activity assay, using the reaction mixture containing 100 mM Tris-HCl (pH 7.0), 80 mM KCl, 2 mM EGTA and 2 mM EDTA, 0.4 mM lipid vesicles, and 30 μg of protein in a total volume of 100 μl. Lipid vesicles were composed of 35 mM of PE, 3 mM PIP₂, and 2 mM PC containing dipalmitoylglycero-3-phospho[methyl-³H]choline as substrate⁹. The reaction was initiated by adding microsomal proteins and was incubated at 30° C. for 30 min in a shaking water bath. The reactions were stopped by adding 1 ml of chloroform:methanol (2:1) and then 100 μl of 2 M KCl, and the release of [³H]choline into the aqueous phase was quantified by scintillation counting.

Real-Time PCR.

Total RNA was isolated using a rapid cetyl-trimethyl-ammonium bromide method²¹. RNA was digested with RNase-free DNase I, and the absence of genomic DNA contamination was confirmed by PCR, using the treated RNA without reverse transcription (RT). The first-strand cDNA was synthesized from 1 μg of total RNA using an iScript cDNA synthesis kit in a total reaction volume of 20 μL according to the manufacturer's instructions (Bio-Rad). The efficiency of the cDNA synthesis was assessed by real-time PCR amplification of a control gene encoding UBQ10 (At4g05320), and the UBQ10 gene threshold cycle (Cₜ) value was 20±0.5. Only cDNA preparations that yielded similar Ct values for the control genes were used for determination of gene expression. The level of individual gene expression was normalized to that of UBQ10 by subtracting the Ct value of UBQ10 from the tested genes. PCR was performed with a MyiQ sequence detection system (Bio-Rad) using SYBR green to monitor double-stranded DNA synthesis. Each reaction contained 7.5 μL 2×SYBR green master mix reagent, 1.0 ng cDNA, and 200 nM of each gene-specific primer in a final volume of 15 μL. Primers for each gene are listed in Table E1.

TABLE E1
Real Time PCR primers

| | Primer | | |
|---|---|---|---|
| Gene | Sequences | SEQ.ID. NO. | Gene |
| DGAT1 | At2g19450 | Forward 5′-GGTTCATCTTCTGCATTTTCGGA-3′ | SEQ.ID. NO. 20 |
| | | Reverse 5′-TTTTCGGTTCATCAGGTCGTGGT-3′ | SEQ.ID. NO. 21 |
| DGAT2 | At3g51520 | Forward 5′-TGTTTGAGAGGCACAAGTCCCGA-3′ | SEQ.ID. NO. 22 |
| | | Reverse 5′-AGTCCAAATCCAGCTCCAAGGTA-3′ | SEQ.ID. NO. 23 |
| PDAT1 | At5g13640 | Forward 5′-GGAGTGGGGATACCAACGGAACG-3′ | SEQ.ID. NO. 24 |
| | | Reverse 5′-GAAAGGGGATGCAACTGTCGGGA-3′ | SEQ.ID. NO. 25 |
| PDAT2 | At3g44830 | Forward 5′-CTAAGATGATGAGACGAGCCGAA-3′ | SEQ.ID. NO. 26 |
| | | Reverse 5′-ATCTCTCGGTCGGAATCCCTACT-3′ | SEQ.ID. NO. 27 |
| CCT1 | At2g32260 | Forward 5′-GCCACTTCTACTAAACTCCC-3′ | SEQ.ID. NO. 28 |
| | | Reverse 5′-CACACACAAACAAACACATC-3′ | SEQ.ID. NO. 29 |
| CCT2 | At4g15130 | Forward 5′-CTGACGATTTCCAAAGACAA-3′ | SEQ.ID. NO. 30 |
| | | Reverse 5′-TTCAATCCCTTTGTTGCTCA-3′ | SEQ.ID. NO. 31 |
| AAPT1 | At1g13560 | Forward 5′-GCCCTTGGAATCTACTGCTT-3′ | SEQ.ID. NO. 32 |
| | | Reverse 5′-ACATAACTTCACCTATCCTG-3′ | SEQ.ID. NO. 33 |
| AAPT2 | At3g25585 | Forward 5′-CGAACCAAAAGGATTGAAAA-3′ | SEQ.ID. NO. 34 |
| | | Reverse 5′-TCCACAAGAGGAACCCCGTC-3′ | SEQ.ID. NO. 35 |
| PLDζ1 | At3g16785 | Forward 5′-TGGATGGCAACCGCAAAGACAA-3′ | SEQ.ID. NO. 36 |
| | | Reverse 5′-ATCGTTGTGTGTCCCAGCTTCT-3′ | SEQ.ID. NO. 37 |
| PLDζ2 | At3g05630 | Forward 5′-TTTGAGGACGGTCCAATTGCCA-3′ | SEQ.ID. NO. 38 |
| | | Reverse 5′-ACAACACCGATCTCAGAGTCTCGT-3′ | SEQ.ID. NO. 39 |
| UBQ10 | At4g05320 | Forward 5′-CACACTCCACTTGGTCTTGCGT-3′ | SEQ.ID. NO. 40 |
| | | Reverse 5′-TGGTCTTTCCGGTGAGAGTCTTCA-3′ | SEQ.ID. NO. 41 |

The following standard thermal profile was used for all PCRs: 95° C. for 3 min; and 50 cycles of 95° C. for 30 s, 55° C. for 30 s, and 72° C. for 30 s.

Fatty Acid Composition and Oil Content.

Dried Arabidopsis or camelina seeds (10-30 mg/sample) were placed in glass tubes with Teflon-lined screw caps, 1.5 ml 5% (v/v) H₂SO₄ in MeOH, and 0.2% butylated hydroxyl toluene. The tubes were incubated at 90° C. for 2 h for oil extraction and transmethylation. Fatty acid methyl esters (FAMEs) were extracted with hexane. For soybean seed oil quantification, a small portion of cotyledon tissue was taken from the opposite side of the seed embryo. The oil in the weighed soybean chips was extracted and transmethylated as previously described. The remaining portion of the seeds were grown for further analysis. FAMEs were quantified using gas chromatography supplied with a hydrogen flame ionization detector and a capillary column SUPELCOWAX-10 (30 m; 0.25 mm) with He carrier at 20 ml/min. The oven temperature was maintained at 170° C. for 1 min and then increased in steps to 210° C., raising the temperature by 3° C. every min FAMEs from TAG were identified by comparing their retention times with known standards. Heptacanoic acid (17:0) was used as the internal standard to quantify the amounts of individual lipids. The statistical significance was evaluated by t-test.

Phospholipid Analysis.

Seeds from siliques at different days after flowering were collected under an anatomic microscope except for the very early stage in which whole silique was used for lipid extraction. Half of the seeds or siliques were used for lipid extraction and the other half were dried in an oven at 105° C. overnight and weighed for dry weight. The procedures for extraction, analysis, and quantification of lipids were performed as described²⁹. Briefly, developing seeds or siliques collected at specified stages were immersed immediately into 3 ml 75° C. isopropanol with 0.01% butylated hydroxytoluene to inhibit lipolytic activities. Phospholipids were separated by TLC (Silica Gel 60; Merck) with the solvent of chloroform/methanol/acetic acid/water (85:15:12.5:3.5, v/v). TAGs were separated from the total lipids by developing the TLC plates in hexane/diethyl ether/acetic acid (70:30:1; v/v/v). Individual lipids were made visible by spraying the plates with 0.01% primuline in acetone/H₂O (60:40; v/v) and examining the plates under ultraviolet (360 nm) light. Lipids were quantified by GC analysis of fatty acid content. A sample of the extracted lipds was used to profile polar glycerolipid sepecies using a tendem mass spectromery-based method³°.

Lipid Visualization by Nile Red.

Lipids in mature seeds was visualized using the dye nile red, 9-diethylamino-5H-benzo[alpha]phenoxazine-5-one, based on a method previously described. A stock of 1 mg/ml of nile red in acetone was prepared and kept in dark at −20° C., and the solution was diluted 100× in water prior to use. Seed coats were removed from camelina seeds to allow better penetration of the dye. Coatless seeds were incubated in nile red for five minutes before observation under a Zeiss LSM 700 confocal microscope.

Protein Content Determination.

Total protein content in cultivar Jack and transgenic lines was determined by nitrogen analysis. The nitrogen content in soybean was analyzed at Duke Environmental Stable Isotope Laboratory, by using CE Instruments NC 2100 elemental analyzer (ThermoQuest Italia, Milan). 3-4 mg of pulverized seeds were accurately weighed and used. Total nitrogen derived from the analysis is converted into protein by multiplying the nitrogen-protein conversion factor of 6.25.

### Example 1

### Evaluation of Role of PLD Zeta on Oil Seed Oil Accumulation by Creation of Insertional Knock Out Mutants

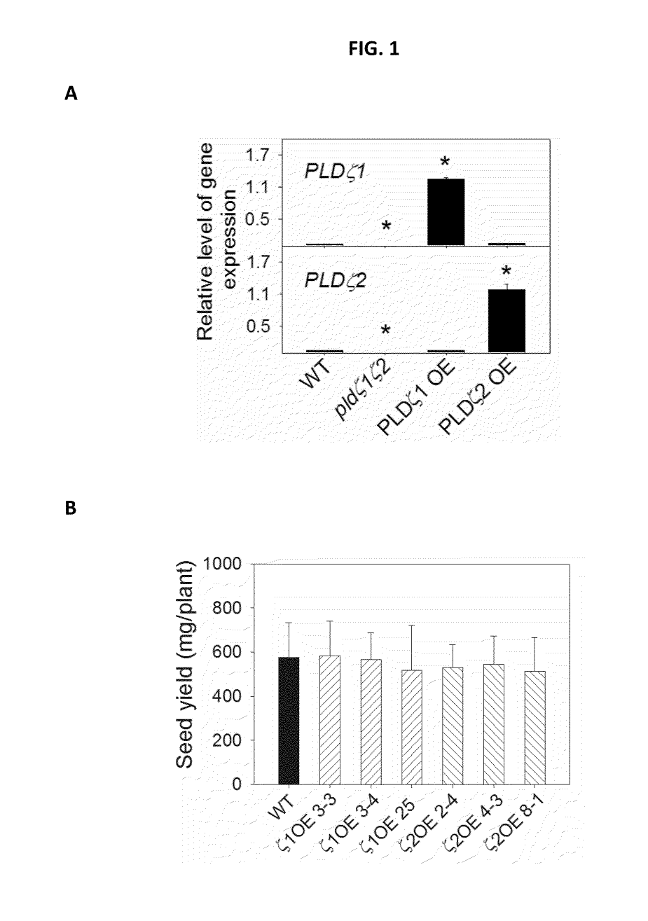

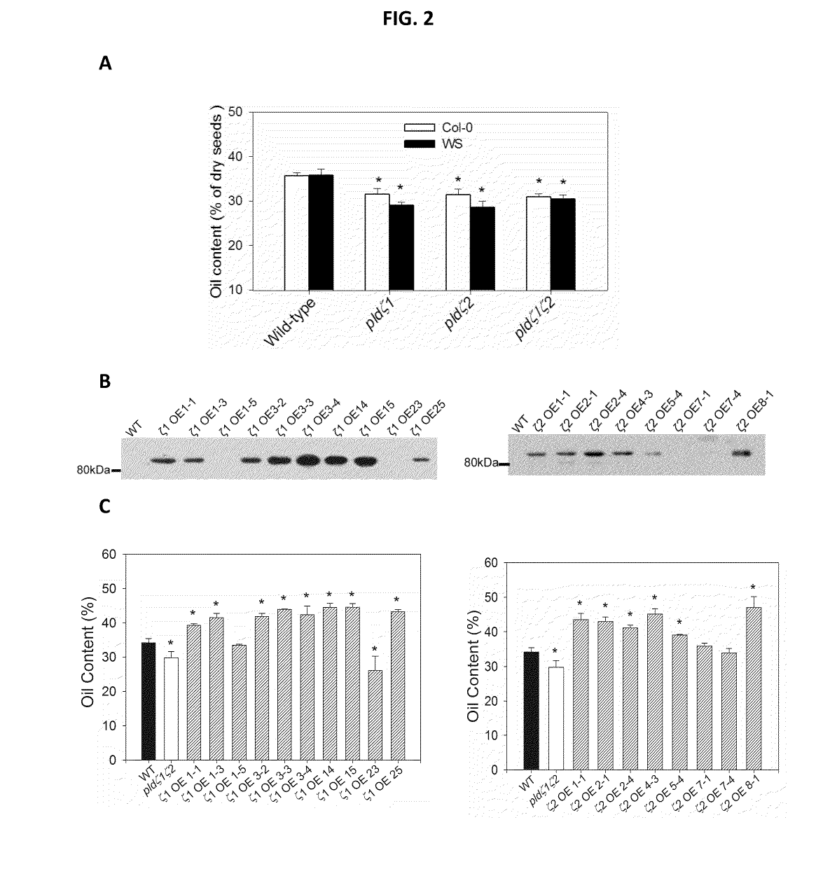

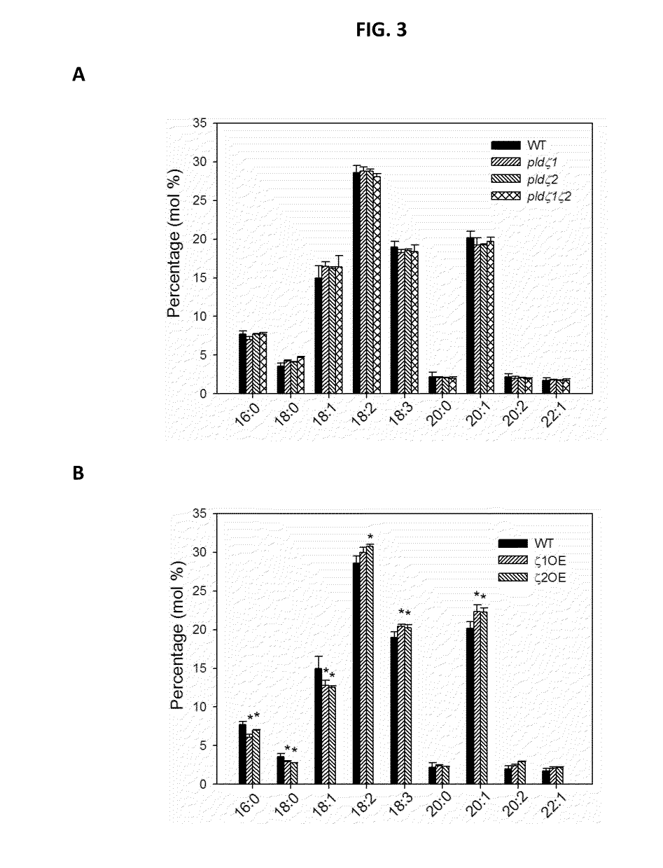

To determine the effect of PLDζs on seed oil accumulation, we isolated T-DNA insertional knockout mutants of PLDζs from two ecotypes: pldζ1-1ws and pldζ2-1ws from Wassilewskija (WS), and pldζ1-1 and pldζ2-1 from Columbia (Col-0). The double knockout mutant pldζ1pldζ2 was produced by crossing PLDζ single mutants²². The expression of PLDζ1 and PLDζ2 was abrogated in the T-DNA insertional PLDζ mutants as confirmed by real-time PCR analysis (FIG. 1). Arabidopsis plants deficient in PLDζ1, ζ2, or both PLDζs grew and developed normally under regular laboratory growth conditions. Ablation of either PLDζ1 or PLDζ2 decreased seed oil content (FIG. 2A). Both Col-0 and WS seeds contained approximately 35% oil, whereas the oil content was 31% for pldζ1-1 and pldζ2-1 seeds, and 29% for pldζ1-1ws and pldζ2-1ws seeds (FIG. 2A). Double knockout of pldζ1pldζ2 did not further decrease seed oil content, suggesting that the effect of PLDζ1 and ζ2 on seed oil content is non-additive (FIG. 2A). Except for a slight increase in stearic acid, fatty acid composition of the PLDζ-KO seeds was comparable to that of WT seeds (FIG. 3). The results suggest that PLD ζ plays an important regulatory role in seed oil production.

### Example 2

### Evaluation of PLD Zeta Over Expression on Oil Seed Oil Accumulation in Arabidopsis

To further examine the role of PLDζs in oil production, PLDζs were specifically in Arabidopsis seeds by placing PLDζ1 and ζ2 cDNAs under the control of the seed-specific promoter of β-conglycinin²³. The presence of transgenic PLDζ protein was detected by immunoblotting with antibodies against the flag tag that was fused to PLDζ1 and PLDζ2 at the C terminus (FIG. 2B). Substantial increases in PLDζ1 and PLDζ2 transcript levels were detected by real-time PCR (FIG. 1). The seed oil content was increased in most of the PLDζ1-Over Expressers or PLDζ2-Over Expresser plants; the increases ranged from 2 to 10% and were associated with the presence of PLDζ-flag protein (FIGS. 2B and C). There was no difference in seed yield per plant between WT and high-oil transgenic lines (FIG. 1B). Seed oils of PLDζ1-OE or PLD2-OE displayed an increase in the relative levels (mol %) of linoleic (18:2), linolenic (18:3), and gondoic (20:1) acids but a decrease in palmitic (16:0), stearic (18:0), and oleic (18:1) when compared to the fatty acid composition of WT seed oils (FIG. 3B).

### Example 3

### Evaluation of PLD Zeta Over Expression on Oil Seed Oil Accumulation in Camelina

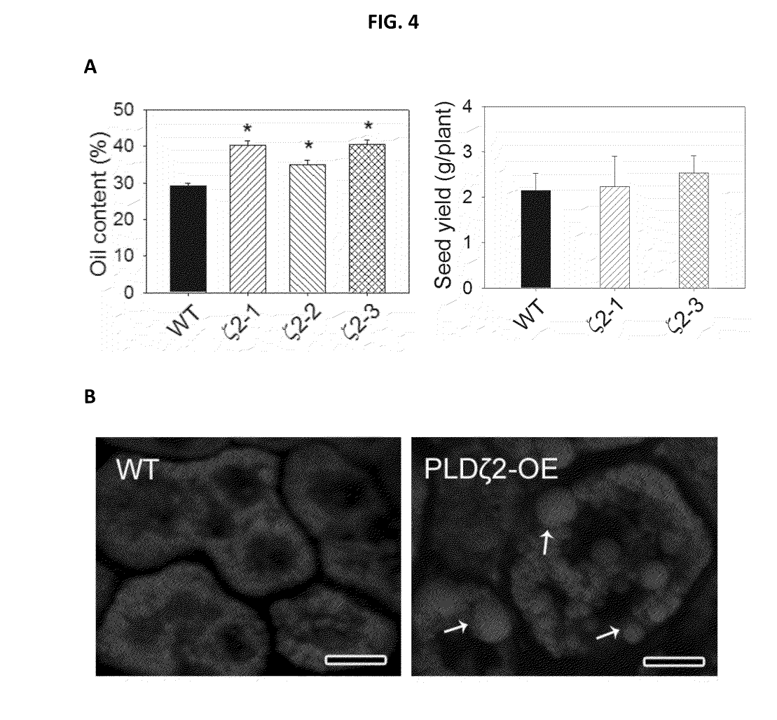

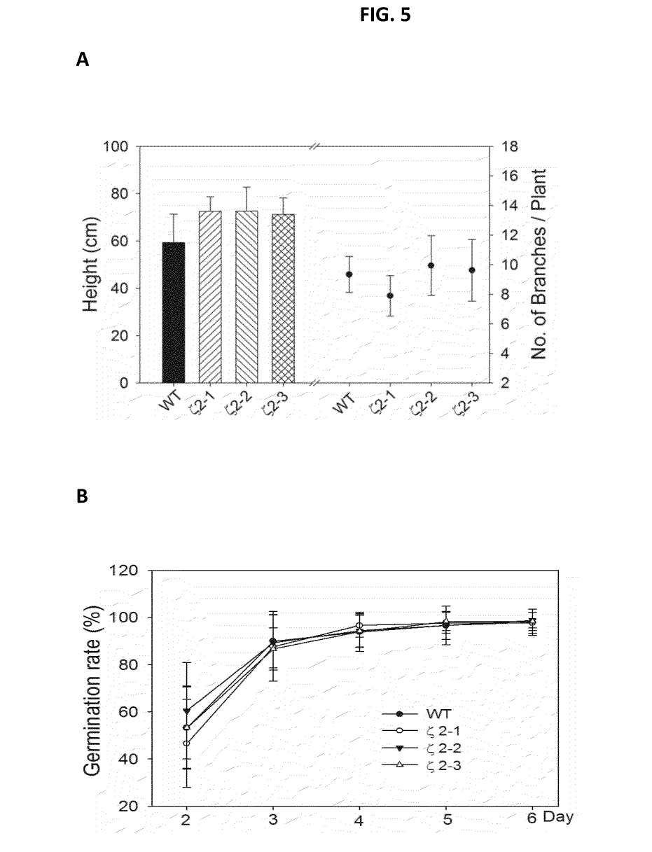

To test whether the effect of PLDζs on oil increases is applicable to other plant species, we transformed the same PLDζ2 gene constructs into Camelina sativa, a promising low-input oilseed crop²⁴. Camelina is a short-seasoned, fast-growing crop that requires less water and fertilizer than many other crops. Seed oil levels in PLDζ2-Over Expressers (OE) were significantly higher than the 30% oil content in untransformed or empty-vector-transformed camelina. The oil content reached 38-40% in some PLDζ2-OE plants, representing an 8-10% increase in oil content (FIG. 4A). When mature seeds were stained with the fluorescent dye nile red (9-diethylamino-5H-benzo(α)phenoxazine-5-one), which has been used for the rapid detection of lipids²⁵, PLDζ2-OE camelina seeds yielded brighter fluorescence and had larger oil bodies than WT seeds (FIG. 4B). Transgenic plants grew and developed normally, and there was no significant difference from WT in total seed weight per plant (FIG. 4C), plant height, numbers of branches, or germination rate (Supplemental FIG. 5).

### Example 4

### Evaluation of PLD Zeta Over Expression on Oil Seed Oil Accumulation in Soybean

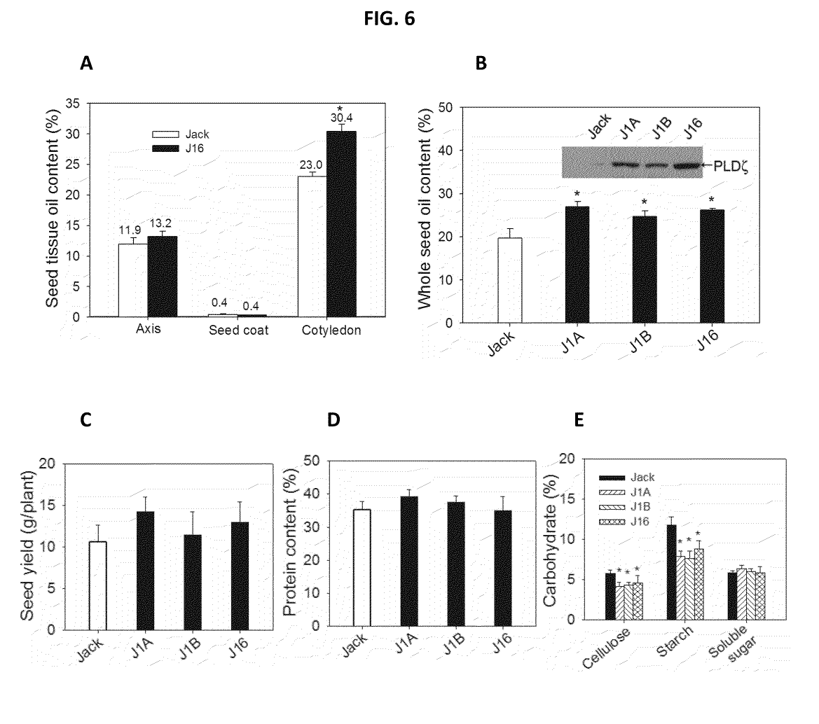

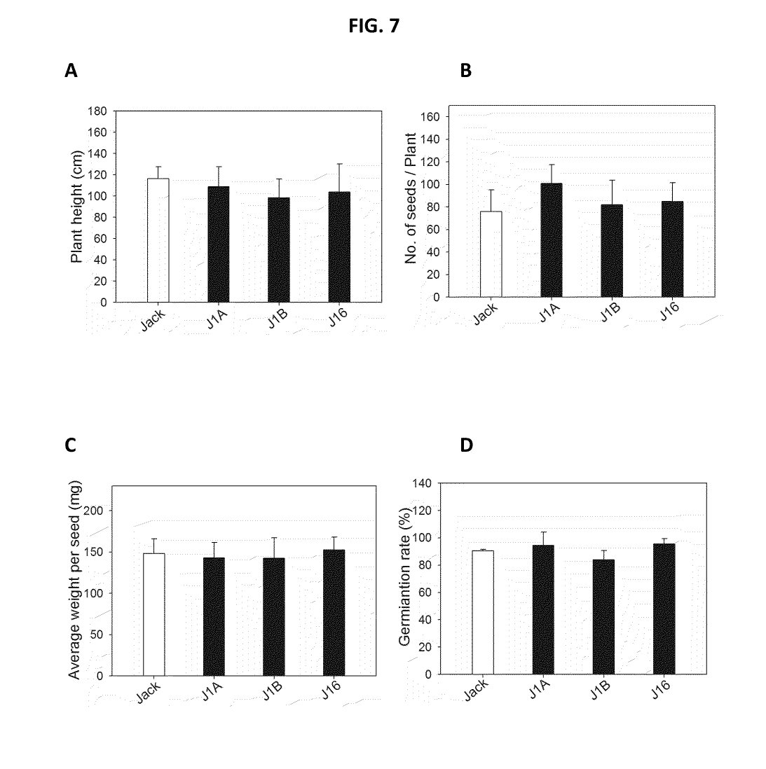

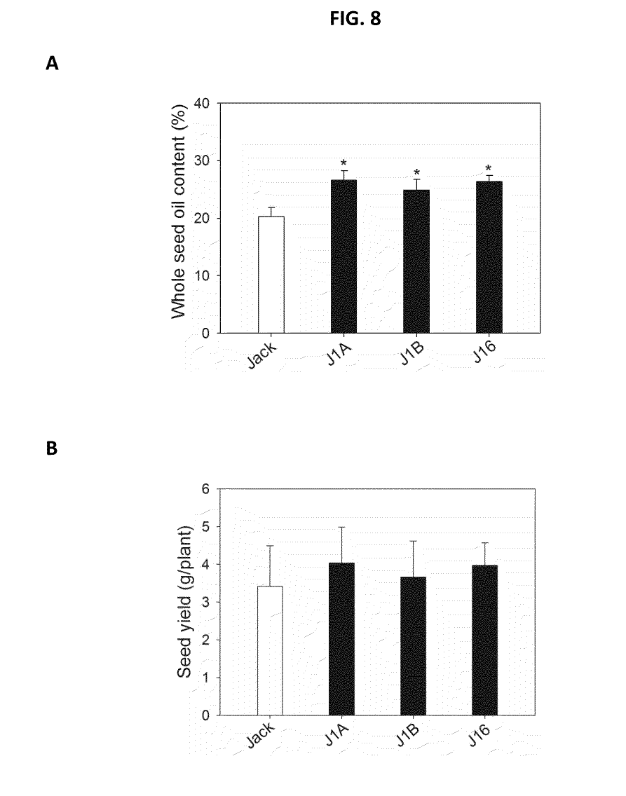

To determined whether the effect of PLDζs over expression on seed oil content could go beyond the Brassicaceae members. The PLDζ seed-specific constructs were transferred into soybean using Agrobacterium-mediated transformation. Multiple transgenic soybean lines carrying PLDζ1 genes were produced and confirmed by their resistance to the herbicide Basta, PCR, and immunoblotting of the introduced PLD (FIG. 6). Oil analysis was performed on individual seeds from T1 plants by taking a small portion of the cotyledon, and the remaining seeds were germinated for next generation confirmation of altered oil content and the presence of the PLD transgene. The high and low oil seeds segregated in a 3:1 ratio in most of PLDζ1-transgenic lines. Soybean plants germinated from the lower end of oil content were sensitive to Basta whereas those from the higher oil content were Basta-resistant and harbored PLDζ transgene. The oil increase was confined to cotyledons, and no increase occurred in seed coat or axis (hypocotyl, radical, and epicotyls; FIG. 6A). The oil content of seed cotyledons for some T1 PLDζ1-OE plants reached 30%, whereas untransformed cultivar Jack cotyledons contained approximately 23% oil (FIG. 6A). In T2 generation, the whole seed oil content in three PLDζ1-OE lines varied from 25-28% whereas that in Jack was 20% (FIG. 6B). The total seed oil content is lower than that of cotyledons because seed coats that have less than 0.5% oil constitute about 8-10% of seed weight. The high oil PLDζ1-OE lines and Jack had similar seed yield as measured by total seed weight per plant (FIG. 6C), seed number, seed weight, and germination rate (FIG. 7). The similar magnitude of oil increase was observed in the T3 generation of seeds from these lines, with no significant difference in seed yield (g/per plant) among these genotypes (FIGS. 8A and B).

One problem plaguing the effort of increasing soy oil content has been the inverse relationship between oil and protein content. Because protein is the major product of soy seeds, improving oil content without compromising protein content is highly desirable. We compared the protein content of the high oil PLDζ1 transgenic lines with that of Jack, and no significant difference was observed (FIG. 6D). When seed carbohydrate content was assessed, the PLDζ1-OE T2 seeds exhibited an approximate decrease of 2% in cellulose and 3.5% in starch, whereas the content of soluble sugar in the transgenic seeds was similar to that of Jack (FIG. 6E). The data indicate that the increase in oil content is primarily at the expense of carbohydrates.

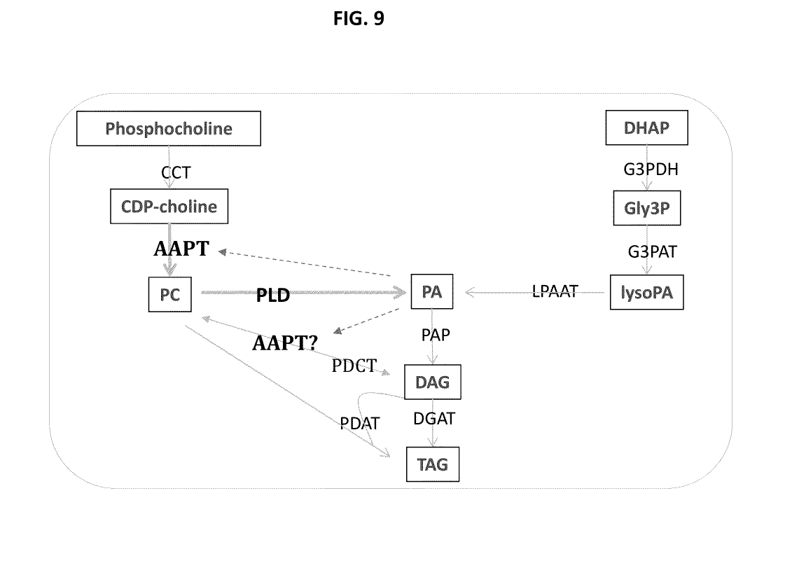

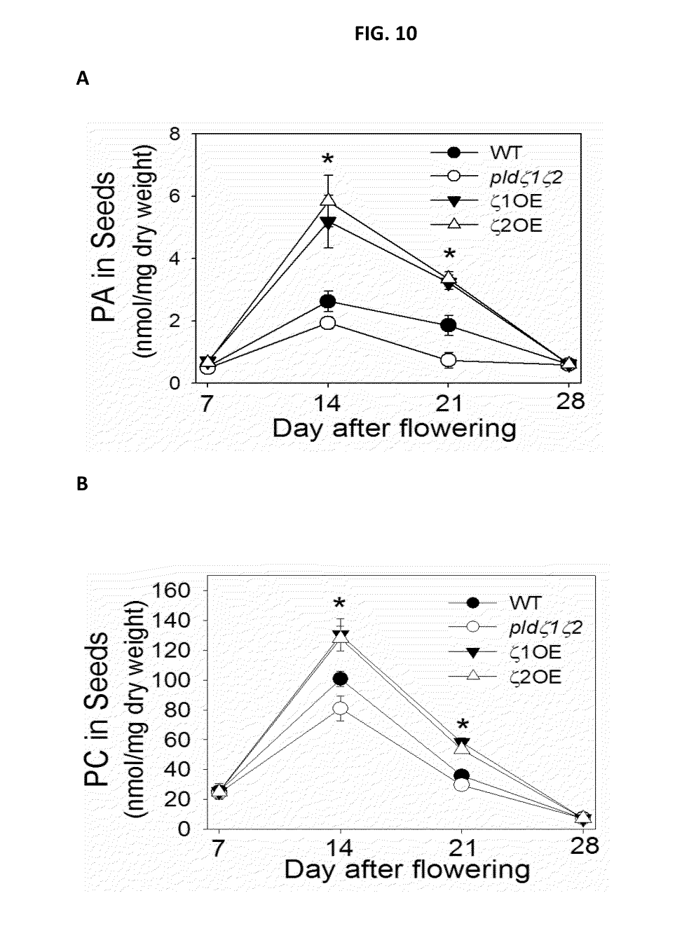

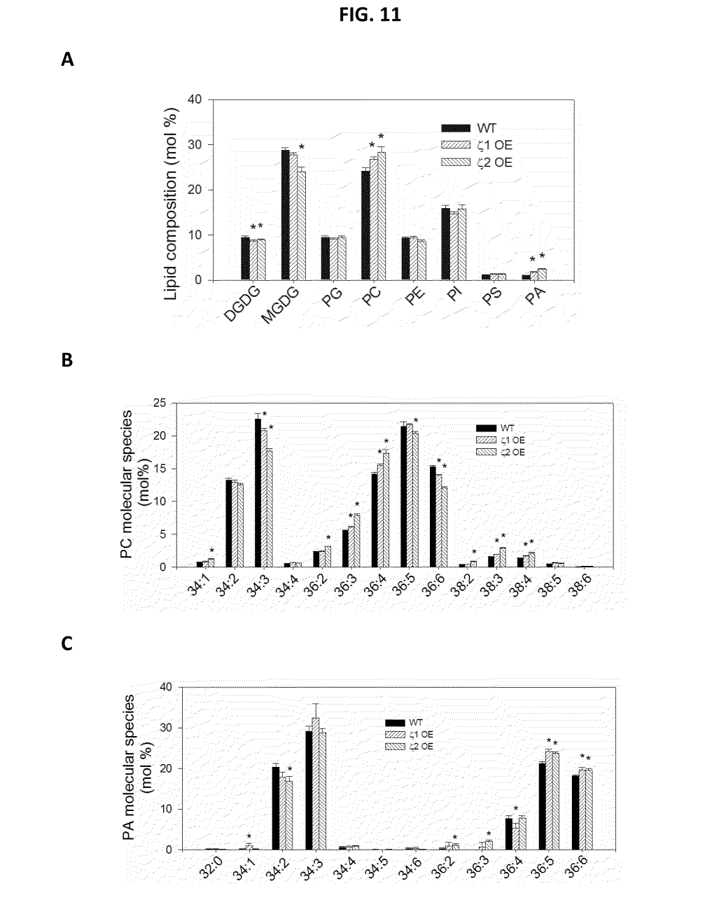

The results from Arabidopsis, camelina, and soybean indicate that PLDζs play important roles in seed oil accumulation. The increased expression of PLDζs may promote the turnover of PC to produce PA that can be dephosphorylated to DAG to produce TAG by DGAT (FIG. 9). Indeed, an increase in PA occurred in developing Arabidopsis seeds. The difference was specific to developmental stages, with the greatest increase at 14 days post flowering (FIG. 10A). Because PLDζ hydrolyzes PC to PA, one might expect that the increase in PA was accompanied by a decrease of PC in PLDζ-OE seeds. However, PC was increased substantially in developing seeds, and the change in PC followed the same trend as PA in developing seeds (FIG. 10B). Profiling membrane lipids by mass spectrometry also revealed a significant increase in PA and PC in developing seeds (FIG. 11). The increase of both PA and PC in PLDζ-OE seeds indicates that the effects of altered PLDζ expression on glycerolipid metabolism are broad, and not limited to the hydrolysis of PC and resulting production of PA.

### Example 5

### Quantification of the Expression of Genes Involved in TAG and PC Biosynthesis

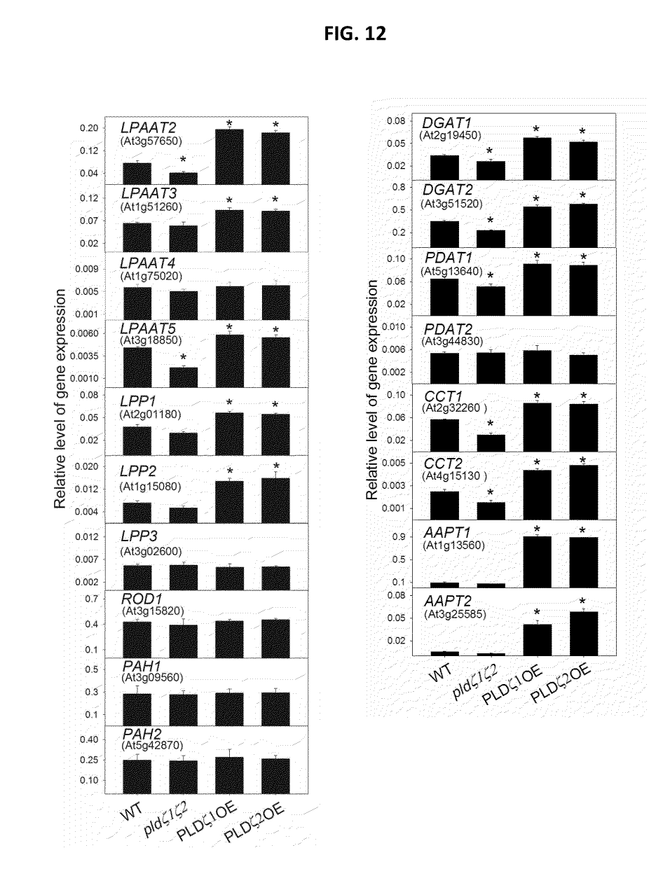

To examine the impact of PLD zeta on the expression of genes involved in TAG and PC biosynthesis was analyzed by real time PCR analysis. The results demonstrated that here were increases in expression for some of the genes in the Kennedy pathways of TAG and PC synthesis, but most noticeable was the approximately ten-fold increase in expression for two aminoalcoholphosphotransferase genes, AAPT1 and AAPT2 in PLDζ-OE Arabidopsis seeds (FIG. 12). AAPTs catalyze the last step of PC biosynthesis by transferring phosphocholine from CDP-choline to DAG (FIG. 9). The increased expression of AAPTs may explain in part the increased PC production. The marked increase in AAPT expression in PLDζ-OE seeds could mean that the PLDζ-derived PA directly or indirectly regulates the expression of AAPT genes. AAPT activity has also been suggested to release DAG for TAG synthesis via its reverse reaction (FIG. 9). PC can also serve as a direct substrate for the production of DAG by PC:DAG cholinephosphotransferase (PDCT)¹⁴ and for TAG biosynthesis by PDAT that transfers the sn-2 acyl chain from PC to DAG, forming lyso-PC and TAG¹² (FIG. 9). Recent studies indicate that fatty acids are incorporated directly into PC by acyl editing¹³. PC then becomes more unsaturated as it acts as substrates for unsaturation to produce polyunsaturated fatty acids. The DAG moiety of PC is combined with a third acyl chain to form most TAG species¹⁵. The increase in 18:2 and 18:3 fatty acids in TAG in PLDζ-OE seeds is consistent with the notion that most fatty acids in TAG come from PC. Thus, increased PC production has multifaceted effects which enhance overall TAG formation (FIG. 9).

The increase in the expression of several genes in the Kennedy pathway for TAG formation (FIG. 12) suggests possible feed-forward stimulation by PC and PA for the TAG biosynthesis. Besides the metabolic effect, PLDζs may have regulatory functions in seed oil production (FIG. 9). PA has been shown to interact directly with a number of proteins including transcriptional factors, protein kinases, and phosphatases^{16, 18, 26}. I yeast, an increase in PA tethers the transcriptional repressor Opilp to the ER, preventing it from reaching the nucleus, thus allowing the expression of genes for phospholipid biosynthesis to increase¹⁶. A recent study in Arabidopsis suggests that PA may also act in a similar way in plants to regulate PC production²⁷. Our results indicate that an increase in PA promotes TAG accumulation. In addition, PLDζs have been implicated in promoting vesicle trafficking in roots and leaves^{20, 28}. Such a role in developing seeds could lead to increase membrane trafficking and lipid body biogenesis, thereby increasing TAG sink and oil deposition.

In summary, the present study has identified PLDζs as novel regulators of seed oil production. We propose that the increased PLDζ expression stimulates PA production, and this PLDζ-derived PA promotes AAPT expression. Thus, PC production and the increases in PA and PC metabolism enhance TAG synthesis and accumulation in seeds (FIG. 9). PLDζ1 and PLDζ2 likely have unique and overlapping functions in the cell and their effects are both metabolic and regulatory. It would be of interest in further studies to identify the direct targets of PA in developing seeds and determine the precise mechanism by which PLD and PA promote AAPT expression and the biosynthesis of PC and TAG. The results with Arabidopsis and the oil crops camelina and soybean suggest that the seed-specific manipulation of PLDζ has the potential to increase seed oil content and vegetable oil production in crops.

While the invention has been described in connection with specific embodiments thereof, it will be understood that the inventive device is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth and as follows in scope of the appended claims.

### REFERENCES

1. Durrett, T. P., Benning, C. & Ohlrogge, J. Plant triacylglycerols as feedstocks for the production of biofuels. Plant J. 54, 593-607 (2008).
2. Weselake, R. J. et al. Increasing the flow of carbon into seed oil. Biotechnol. Adv. 27, 866-878 (2009).
3. Jain, R. K., Coffey, M., Lai, K., Kumar, A. & MacKenzie, S. L. Enhancement of seed oil content by expression of glycerol-3-phosphate acyltransferase genes. Biochem. Soc. Trans. 28, 958-961 (2000).
4. Zou, J. et al. Modification of seed oil content and acyl composition in the brassicaceae by expression of a yeast sn-2 acyltransferase gene. Plant Cell 9, 909-923 (1997).
5. Jako, C. et al. Seed-specific over-expression of an Arabidopsis cDNA encoding a diacylglycerol acyltransferase enhances seed oil content and seed weight. Plant Physiol. 126, 861-874 (2001).
6. Bouvier-Nave, P., Benveniste, P., Oelkers, P., Sturley, S. L. & Schaller, H. Expression in yeast and tobacco of plant cDNAs encoding acyl CoA:diacylglycerol acyltransferase. Eur. J. Biochem. 267, 85-96 (2000).
7. Zheng, P. et al. A phenylalanine in DGAT is a key determinant of oil content and composition in maize. Nat. Genet. 40, 367-372 (2008).
8. Roesler, K., Shintani, D., Savage, L., Boddupalli, S. & Ohlrogge, J. Targeting of the Arabidopsis homomeric acetyl-coenzyme A carboxylase to plastids of rapeseeds. Plant Physiol. 113, 75-81 (1997).
9. Vigeolas, H., Waldeck, P., Zank, T. & Geigenberger, P. Increasing seed oil content in oil-seed rape (Brassica napus L.) by over-expression of a yeast glycerol-3-phosphate dehydrogenase under the control of a seed-specific promoter. Plant Biotechnol. J. 5, 431-441 (2007).
10. Taylor, D. C. et al. Field testing of transgenic rapeseed cv. Hero transformed with a yeast sn-2 acyltransferase results in increased oil content, erucic acid content and seed yield. Mol. Breed. 8, 317-322 (2001).
11. Rao, S. S. & Hildebrand, D. Changes in oil content of transgenic soybeans expressing the yeast SLC1 gene. Lipids 44, 945-951 (2009).
12. Lardizabal, K. et al. Expression of Umbelopsis ramanniana DGAT2A in seed increases oil in soybean. Plant Physiol. 148, 89-96 (2008).
13. Dahlqvist, A. et al. Phospholipid:diacylglycerol acyltransferase: an enzyme that catalyzes the acyl-CoA-independent formation of triacylglycerol in yeast and plants. Proc. Natl. Acad. Sci. USA 97, 6487-6492 (2000).
14. Lu, C., Xin, Z., Ren, Z., Miguel, M. & Browse, J. An enzyme regulating triacylglycerol composition is encoded by the ROD1 gene of Arabidopsis. Proc Natl Acad Sci USA 106, 18837-1842 (2009).
15. Bates, P. D., Durrett, T. P., Ohlrogge, J. B. & Pollard, M. Analysis of acyl fluxes through multiple pathways of triacylglycerol synthesis in developing soybean embryos. Plant Physiol. 150, 55-72 (2009).
16. Carman, G. M. & Henry, S. A. Phosphatidic acid plays a central role in the transcriptional regulation of glycerophospholipid synthesis in Saccharomyces cerevisiae. J. Biol. Chem. 282, 37293-37297 (2007).
17. Awai, K., Xu, C., Tamot, B. & Benning, C. A phosphatidic acid-binding protein of the chloroplast inner envelope membrane involved in lipid trafficking. Proc. Natl. Acad. Sci. USA 103, 10817-10822 (2006).
18. Li, M., Hong, Y. & Wang, X. Phospholipase D- and phosphatidic acid-mediated signaling in plants. Biochim. Biophys. Acta 1791, 927-935 (2009).
19. Qin, C. & Wang, X. The Arabidopsis phospholipase D family. Characterization of a calcium-independent and phosphatidylcholine-selective PLD zeta 1 with distinct regulatory domains. Plant Physiol. 128, 1057-1068 (2002).
20. Ohashi, Y. et al. Modulation of phospholipid signaling by GLABRA2 in root-hair pattern formation. Science 300, 1427-1430 (2003).
21. Shen, B., Sinkevicius, K. W., Selinger, D. A. & Tarczynski, M. C. The homeobox gene GLABRA2 affects seed oil content in Arabidopsis. Plant Mol. Biol. 60, 377-387 (2006).
22. Li, M., Qin, C., Welti, R. & Wang, X. Double knockouts of phospholipases Dζ1 and Dζ2 in Arabidopsis affect root elongation during phosphate-limited growth but do not affect root hair patterning. Plant Physiol. 140, 761-770 (2006).
23. Fujiwara, T. & Beachy, R. N. Tissue-specific and temporal regulation of a beta-conglycinin gene: roles of the RY repeat and other cis-acting elements. Plant Mol. Biol. 24, 261-272 (1994).
24. Gehringer, A., Friedt, W., Luhs, W. & Snowdon, R. J. Genetic mapping of agronomic traits in false flax (Camelina sativa subsp. sativa). Genome 49, 1555-1563 (2006).
25. Greenspan, P., Mayer, E. P. & Fowler, S. D. Nile red: a selective fluorescent stain for intracellular lipid droplets. J. Cell Biol. 100, 965-973 (1985).
26. Zhang, W., Qin, C., Zhao, J. & Wang, X. Phospholipase Dα1-derived phosphatidic acid interacts with ABI1 phosphatase 2C and regulates abscisic acid signaling. Proc. Natl. Acad. Sci. USA 101, 9508-9513 (2004).
27. Eastmond, P. J., Quettier, A. L., Kroon, J. T., Craddock, C., Adams, N. & Slabas, A. R. Phosphatidic acid phosphohydrolase 1 and 2 regulate phospholipid synthesis at the endoplasmic reticulum in Arabidopsis. Plant Cell. 22:2796-2811 (2010).
28. Li, G. & Xue, H. W. Arabidopsis PLDzeta2 regulates vesicle trafficking and is required for auxin response. Plant Cell 19, 281-295 (2007).
29. Lu, C. & Kang, J. Generation of transgenic plants of a potential oilseed crop Camelina sativa by Agrobacterium-mediated transformation. Plant Cell Rep. 27, 273-278 (2008).
30. Zeng, P., Vadnais, D. A., Zhang, Z. & Polacco, J. C. Refined glufosinate selection in Agrobacterium-mediated transformation of soybean [Glycine max (L.) Merrill]. Plant Cell Rep. 22, 478-482 (2004).
31. Wanjie, S. W., Welti, R., Moreau, R. A. & Chapman, K. D. Identification and quantification of glycerolipids in cotton fibers: reconciliation with metabolic pathway predictions from DNA databases. Lipids 40, 773-785 (2005).
32. Welti, R. et al. Profiling membrane lipids in plant stress responses. Role of phospholipase Da in freezing-induced lipid changes in Arabidopsis. J. Biol. Chem. 277, 31994-32002 (2002).
33. Updegraff, D. M. Semimicro determination of cellulose in biological materials. Anal. Biochem. 32, 420-424 (1969).
34. Jones, M. G., Outlaw, W. H. & Lowry, O. H. Enzymic assay of 10⁻⁷ to 10⁻¹⁴ moles of sucrose in plant tissues. Plant Physiol. 60, 379-383 (1977).
35. Eimert, K., Wang, S. M., Lue, W. I. & Chen, J. Monogenic recessive mutations causing both late floral initiation and excess starch accumulation in Arabidopsis. Plant Cell 7, 1703-1712 (1995).

1-30. (canceled)

31. A method for increasing plant seed oil content, comprising overexpressing one or multiple PLD zeta family enzymes in a seed.

32. The method of claim 31, wherein said PLD zeta family enzyme is selected from PLDζ1, PLDζ2, or a combination thereof.

33. The method of claim 31, wherein said PLD zeta family enzyme comprises an amino acid sequence selected from the group consisting of a sequence having at least 99% sequence identity to SEQ ID NO:1, a sequence having at least 99% sequence identity to SEQ ID NO:2, SEQ ID NO:3, SEQ ID NO:4, SEQ ID NO:5, and SEQ ID NO:6.

34. The method of claim 31, wherein said one or multiple PLD zeta family enzymes is expressed under the control of a promoter that drives PLD zeta expression in a seed.

35. The method of claim 34, wherein said promoter is selected from the group consisting of β-conglycinin promoter, soybean oleosin promoter, and rapeseed napin promoter.

36. The method of claim 31, wherein said seed is a seed of Arabidopsis, Camelina, or soybean.

37. A method for producing seed oil, comprising transforming a plant cell with a nucleotide sequence encoding a PLD zeta enzyme operatively linked to expression control sequences that drive expression of said nucleotide sequence in said plant cell.

38. The method of claim 37, wherein said PLD zeta enzyme comprises an amino acid sequence selected from the group consisting of a sequence having at least 99% sequence identity to SEQ ID NO:1, a sequence having at least 99% sequence identity to SEQ ID NO:2, SEQ ID NO:3, SEQ ID NO:4, SEQ ID NO:5, and SEQ ID NO:6.

39. The method of claim 37, wherein said expression control sequences comprise a cell type specific promoter.

40. The method of claim 37, wherein said expression control sequences comprise a seed specific promoter.

41. The method of claim 40, wherein said seed specific promoter is selected from the group consisting of soybean oleosin promoter, rapeseed napin promoter, and β-conglycinin promoter.

42. The method of claim 37, wherein said plant cell is a monocotyledonous plant cell.

43. The method of claim 37, wherein said plant cell is a dicotyledonous plant cell.

44. The method of claim 43, wherein said dicotyledonous plant cell is selected from the group consisting of a Camelina plant cell, an Arabidopsis plant cell, and a soybean plant cell.

45. The method of claim 37, further comprising regenerating a stably transformed transgenic plant from said transformed plant cell.

46. The method of claim 45, further comprising growing said transgenic plant and harvesting seeds thereof.

47. The method of claim 46, further comprising recovering oil from said harvested seeds.

48. Oil recovered by the method of claim 47.

49. A transgenic plant, comprising a cell having within its genome a heterologous nucleotide sequence encoding a protein comprising a PLD zeta enzyme, operatively linked to expression control sequences that drive expression of said heterologous nucleotide sequence in said plant cell.

50. The transgenic plant of claim 49, wherein said PLD zeta enzyme comprises an amino acid sequence selected from the group consisting of a sequence having at least 99% sequence identity to SEQ ID NO:1, a sequence having at least 99% sequence identity to SEQ ID NO:2, SEQ ID NO:3, SEQ ID NO:4, SEQ ID NO:5, and SEQ ID NO:6.

51. The transgenic plant of claim 49, wherein said expression control sequences comprise a cell type specific promoter.

52. The transgenic plant of claim 49, wherein said expression control sequences comprise a seed specific promoter.

53. The transgenic plant of claim 52, wherein said seed specific promoter is selected from the group consisting of soybean oleosin promoter, rapeseed napin promoter, and beta conglycinin promoter.

54. The transgenic plant of claim 49, which is a monocotyledonous plant.

55. The transgenic plant of claim 49, which is a dicotyledonous plant.

56. The transgenic dicotyledonous plant of claim 55, which is selected from the group consisting of Camelina, Arabidopsis, and soybean.

57. The transgenic plant of claim 49, wherein said heterologous nucleotide sequence is expressed primarily in a seed of said transgenic plant.

58. The transgenic plant of claim 49, having an increased seed oil content compared to that of a corresponding wild type, non-transgenic plant grown under similar conditions.

59. The transgenic plant of claim 49, having an increase in the relative levels (mol %) of linoleic (18:2), linolenic (18:3), and gondoic (20:1) fatty acids compared to the levels thereof of a corresponding wild type, non-transgenic plant grown under similar conditions.

60. A seed of said transgenic plant of claim 59.

61. Oil recovered from said seed of claim 60.


SEQUENCE LISTING (41 sequences)

SEQ ID NO: 1 - protein, 1096 aa, Artificial Sequence
  Synthesized
MASEQLMSPASGGGRYFQMQPEQFPSMVSSLFSFAPAPTQETNRIFEELPKAVIVSVSRPDAGDISPVLLSYTIECQYKQFKWQLVKKASQVFYLHFALKKRAFIEEIHEKQEQVKEWLQNLGIGDHPPVVQDEDADEVPLHQDESAKNRDVPSSAALPVIRPLGRQQSISVRGKHAMQEYLNHFLGNLDIVNSREVCRFLEVSMLSFSPEYGPKLKEDYIMVKHLPKFSKSDDDSNRCCGCCWFCCCNDNWQKVWGVLKPGFLALLEDPFDAKLLDIIVFDVLPVSNGNDGVDISLAVELKDHNPLRHAFKVTSGNRSIRIRAKNSAKVKDWVASINDAALRPPEGWCHPHRFGSYAPPRGLTDDGSQAQWFVDGGAAFAAIAAAIENAKSEIFICGWWVCPELYLRRPFDPHTSSRLDNLLENKAKQGVQIYILIYKEVALALKINSVYSKRRLLGIHENVRVLRYPDHFSSGVYLWSHHEKLVIVDNQVCFIGGLDLCFGRYDTFEHKVGDNPSVTWPGKDYYNPRESEPNTWEDALKDELERKKHPRMPWHDVHCALWGPPCRDVARHFVQRWNYAKRNKAPYEDSIPLLMPQHHMVIPHYMGRQEESDIESKKEEDSIRGIRRDDSFSSRSSLQDIPLLLPHEPVDQDGSSGGHKENGTNNRNGPFSFRKSKIEPVDGDTPMRGFVDDRNGLDLPVAKRGSNAIDSEWWETQDHDYQVGSPDETGQVGPRTSCRCQIIRSVSQWSAGTSQVEESIHSAYRSLIDKAEHFIYIENQFFISGLSGDDTVKNRVLEALYKRILRAHNEKKIFRVVVVIPLLPGFQGGIDDSGAASVRAIMHWQYRTIYRGHNSILTNLYNTIGVKAHDYISFYGLRAYGKLSEDGPVATSQVYVHSKIMIVDDRAALIGSANINDRSLLGSRDSEIGVLIEDTELVDSRMAGKPWKAGKFSSSLRLSLWSEHLGLRTGEIDQIIDPVSDSTYKEIWMATAKTNTMIYQDVFSCVPNDLIHSRMAFRQSLSYWKEKLGHTTIDLGIAPEKLESYHNGDIKRSDPMDRLKAIKGHLVSFPLDFMCKEDLRPVFNESEYYASPQVFH

SEQ ID NO: 2 - protein, 1046 aa, Artificial Sequence
  Synthesized
MSTDKLLLPNGVKSDGVIRMTRADAAAAAASSSLGGGSQIFDELPKAAIVSVSRPDTTDFSPLLLSYTLELQYKQFKWTLQKKASQVLYLHFALKKRLIIEELHDKQEQVREWLHSLGIFDMQGSVVQDDEEPDDGALPLHYTEDSIKNRNVPSRAALPIIRPTIGRSETVVDRGRTAMQGYLSLFLGNLDIVNSKEVCKFLEVSRLSFAREYGSKMKEGYVTVKHLRDVPGSDGVRCCLPTHCLGFFGTSWTKVWAVLKPGFLALLEDPFSGKLLDIMVFDTLGLQGTKESSEQPRLAEQVKEHNPLRFGFKVTSGDRTVRLRTTSSRKVKEWVKAVDEAGCYSPHRFGSFAPPRGLTSDGSQAQWFVDGHTAFEAIAFAIQNATSEIFMTGWWLCPELYLKRPFEDHPSLRLDALLETKAKQGVKIYILLYKEVQIALKINSLYSKKRLQNIHKNVKVLRYPDHLSSGIYLWSHHEKIVIVDYQVCFIGGLDLCFGRYDTAEHKIGDCPPYIWPGKDYYNPRESEPNSWEETMKDELDRRKYPRMPWHDVHCALWGPPCRDVARHFVQRWNHSKRNKAPNEQTIPLLMPHHHMVLPHYLGTREIDIIAAAKPEEDPDKPVVLARHDSFSSASPPQEIPLLLPQETDADFAGRGDLKLDSGARQDPGETSEESDLDEAVNDWWWQIGKQSDCRCQIIRSVSQWSAGTSQPEDSIHRAYCSLIQNAEHFIYIENQFFISGLEKEDTILNRVLEALYRRILKAHEENKCFRVVIVIPLLPGFQGGIDDFGAATVRALMHWQYRTISREGTSILDNLNALLGPKTQDYISFYGLRSYGRLFEDGPIATSQIYVHSKLMIVDDRIAVIGSSNINDRSLLGSRDSEIGVVIEDKEFVESSMNGMKWMAGKFSYSLRCSLWSEHLGLHAGEIQKIEDPIKDATYKDLWMATAKKNTDIYNQVFSCIPNEHIRSRAALRHNMALCKDKLGHTTIDLGIAPERLESCGSDSWEILKETRGNLVCFPLQFMCDQEDLRPGFNESEFYTAPQVFH

SEQ ID NO: 3 - protein, 1097 aa, Artificial Sequence
  Synthesized
MASEQLMSPASGGGGRYFQMQPEQFPSMVSSLFSFAPAPTQESNRIFEELPKAVIVSVSRPDAGDISPVLLSYTIECQYKQFKWQLVKKASQVFYLHFALKKRAFIEEIHEKQEQVKEWLQNLGIGDHAPVVQDEDADEVPLHQDESAKNRDVPSSAALPVIRPLGRQQSISVRGKHAMQEYLNHFLGNLDIVNSREVCRFLEVSMLSFSPEYGPKLKEDYIMVKHLPKFSKSDDDSNRCCGCCWFCCCNDNWQKVWGVLKPGFLALLEDPFDAKLLDIIVFDVLPVSNGNDGVDVSLAVELKDHNPLRHAFKVTSGNRSIRIRAKSSAKVKDWVASINDAALRPPEGWCHPHRFGSYAPPRGLTDDGSQAQWFVDGGAAFAAIAAAIENAKSEIFICGWWVCPELYLRRPFDPHTSSRLDNLLENKAKQGVQIYILLYKEVALALKINSVYSKRRLLGIHENVRVLRYPDHFSSGVYLWSHHEKLVIVDNQVCFIGGLDLCFGRYDTFEHKVGDNPSVTWPGKDYYNPRESEPNTWEDALKDELNRKKHPRMPWHDVHCALWGPPCRDVARHFVQRWNYAKRNKAPYEDSIPLLMPQHHMVIPHYMGRQEESDTESKKDEDSIKGIRRDDSFSSRSSLQDIPLLLPQEPVDQDGSSRGHKENGTNNRNGPFSFRKLKIEPVDGDTPMRGFVDDRNGLDLPVAKRGSNAIDSEWWETQEHDYQVGSPDETGQVGPRTSCRCQIIRSVSQWSAGTSQVEESIHSAYRSLIDKAEHFIYIENQFFISGLSGDDTIKNRILEALYKRILRAHNEKKSFRVVVVIPLLPGFQGGIDDSGAASVRAIMHWQYRTIYRGHNSILTNLYNTIGAKAHDYISFYGLRAYGKLSEDGPVATSQVYVHSKIMIIDDRAALIGSANINDRSLLGSRDSEIGVLIEDTEFVDSRMAGKPWKAGKFSSSLRLSLWSEHLGLRTGEIDQIIDPVSDSTYKEIWMATAKTNTMIYQDVFSCVPNDLIHSRMAFRQSLSYWKEKLGHTTIDLGIAPEKLESYHNGDIKRSDPMDRLKSIKGHLVSFPLDFMCKEDLRPVFNESEYYASPQVFH

SEQ ID NO: 4 - protein, 1098 aa, Artificial Sequence
  Synthesized
MASEDLMSGAGARYIQMQSEPMPSTISSFFSFRQSPESTRIFDELPKATIVFVSRPDASDISPALLTYTIEFRYKQFKWRLIKKASQVFFLHFALKKRVIIEEIQEKQEQVKEWLQNIGIGEHTAVVHDDDEPDEETVPLHHDESVKNRDIPSSAALPIIRPALGRQNSVSDRAKVAMQGYLNLFLGNLDIVNSREVCKFLEVSKLSFSPEYGPKLKEDYVMVKHLPKIPKEDDTRKCCPCPWFSCCNDNWQKVWAVLKPGFLALLEDPFHPQPLDIIVFDLLPASDGNGEGRLSLAKEIKERNPLRHALKVTCGNRSIRLRAKSSAKVKDWVAAINDAGLRPPEGWCHPHRFGSFAPPRGLSEDGSLAQWFVDGRAAFEAIASAIEEAKSEIFICGWWVCPELYLRRPFHSHASSRLDALLEAKAKQGVQIYILLYKEVALALKINSVYSKRKLLSIHENVRVLRYPDHFSTGVYLWSHHEKLVIVDYQICFIGGLDLCFGRYDTLEHKVGDHPPLMWPGKDYYNPRESEPNSWEDTMKDELDRGKYPRMPWHDVHCALWGPPCRDVARHFVQRWNYAKRNKAPNEQAIPLLMPQQHMVIPHYMGRSREMEVEKKNVENNYKDIKKLDSFSSRSSFQDIPLLLPQEPDGLDSPHGESKLNGRSLSFSFRKSKIEPVPDMPMKGFVDDLDTLDLKGKMSSDIMAQPGMRTCDREWWETQERGNQVLSADETGQVGPCVPCRCQVIRSVSQWSAGTSQVEDSTHNAYCSLIEKAEHFIYIENQFFISGLSGDEIIRNRVLEVLYRRIMQAYNDKKCFRVIIVIPLLPGFQGGLDDGGAASVRAIMHWQYRTICRGNNSILQNLYDVIGHKTHDYISFYGLRAYGRLFDGGPVASSQVYVHSKIMIVDDCTTLIGSANINDRSLLGSRDSEIGVLIEDKELVDSYMGGKPKKAGKFAHSLRLSLWSEHLGLRGGEIDQIKDPVVDSTYRDVWMATAKTNSTIYQDVFSCIPNDLIHSRAAMRQHMAIWKEKLGHTTIDLGIAPMKLESYDNGDMKTIEPMERLESVKGHLVYFPLDFMCKEDLRPVFNESEYYASPQVFH

SEQ ID NO: 5 - protein, 1117 aa, Artificial Sequence
  Synthesized
MASSEQLMNGSNGPRYVQMQSEPSTPQHNQQQLQQQHPSSMLSSFFSFTHGVTPESTRIFDELPTATIVSVSRPDAGDISPVLLTYTIEFKWQLSKKAAQVFYLHFALKRRAFFEEIHEKQEQVKEWLQNLGIGDHTPVVQDDDDADDETILLHNEESAKNRNVPSRAALPVIRPALGRQHSMSDRAKVAMQEYLNHFLGNLDIVNSREVCKFLEVSKLSFSLEYGPKLKEDYVMARHLPPIPTNDDSGKCCACHWFSCCNDNWQKVWAVLKPGFLALLADPFDAKPLDIIVFDVLPASDGSGEGRISLAMETKERNPLRHAFKVTCGVRSIKLRTKTGARVKDWVAAINDAGLRPPEGWCHPHRFGSFAPPRGLTEDGSQAQWFIDGMAAFDAIASSIEDAKSEIFICGWWLCPELYLRRPFHAHASSRLDDLLEAKAKQGVQIYILLYKEVALALKINSVYSKRKLLSIHENVRVLRYPDHFSSGVYLWSHHEKLVIVDYQICFIGGLDLCFGRYDTREHRVGDCPPFVWPGKDYYNPRESEPNSWEDTMKDELDRKKYPRMPWHDVHCALWGPPCRDVARHFVQRWNYAKRNKAPYEEAIPLLMPQHHMVIPHYRGSSKDLEVETKNGEDDSKGIKREDSFSSRSSLQDIPLLLPQEAEGTDGSGRGPKLNGLDSTPGRSRSYAFRKSKFEAVVPDTPMKGFVDDHNILDLHVKISPDILPQSGTKTSHLEWWETQERGDQVGFGDETGQVGPRTSCRCQVIRSVSQWSAGTSQVEESIHCAYRSLIEKAEHFIYIENQFFISGLSGDEIIRNRVLESLYRRIMRAHNEKKCFRVIIVIPLIPGFQGGLDDSGAASVRAIMHWQYRTICRGQNSIFHNLYDVLGPKTHDYISFYGLRAYGKLFDGGPVATSQVYVHSKIMIIDDCATLIGSANINDRSLLGSRDSEIAVLIEDKEMVDSFMGGRHWKAGKFSLSLRLSLWSEHLGLNAKEMKQIIDPVIDSTYKDIWIATAKTNTTIYQDVFSCIPNDLMHSRAALRQNMAFWKERLGHTTIDLGIAPEKLESYENGDIKKHDPMERLQAVRGHLVSFPLDFMCREDLRPVFNESEYYASQVFY

SEQ ID NO: 6 - protein, 1096 aa, Artificial Sequence
  Synthesized
MQSEPSTPLQPPSSSIISSFFSFRQGSTPESGRIFDELPQATIVSVSRPDPSDISPVQLSYTIEVQYKQFKWRLLKKAAQVFYLHFALKKRVFFEEILEKQEQVKEWLQNLGIGDHTPMVNDDDDADDETIPLHHDESAKNRDVPSSAALPVIRPALGRQNSMSDRAKVTMQQYLNHFLGNMDIVNSREVCKFLEVSKLSFSPEYGPKLKEEYVMVKHLPRIVKDDDSRKCCACSWFSCCNDNWQKVWAVLKPGFLALLADPFDTKLLDIIVFDVLPASDGSGEGRVSLAAEIKERNPLRHGFKVACGNRSIDLRSKNGARVKDWVATINDAGLRPPEGWCHPHRFASFAPPRGLSEDGSQAQWFVDGRAAFEAIALSIEDAKSEIFICGWWLCPELYLRRPFRAHASSRLDSLLEAKAKQGVQIYILLYKEVALALKINSVYSKTKLLSIHENVRVLRYPDHFSTGVYLWSHHEKLVIVDHQICFIGGLDLCFGRYDTCEHRVGDCPPQVWPGKDYYNPRESEPNSWEDMMKDELDRGKYPRMPWHDVHCALWGPPCRDVARHFVQRWNYAKRSKAPYEEAIPLLMPQQHMVIPHYMGQNREMEVERKGIKDDVKGIKRQDSFSSRSSLQDIPLLLPQEAEGPDDSGVGPKLNGMDSTPGRSLPHAFWKSKIELVVPDISMTSFVDNNGSDLHVKMSSDFSAQPGTKASDLEWWETQERVDQVGSPDESGQVGPRVSCHCQVIRSVSQWSAGTSQIEESIHCAYCSLIEKAEHFVYIENQFLISGLSGDDIIRNRVLEALYRRIMRAFNDKKCFRVIIVIPLLPGFQGGVDDGGAASVRAIMHWQYRTICRGQNSILHNLYDHLGPKTHDYISFYGLRSYGRLFDGGPVATSQVYVHSKIMIIDDRTTLIGSANINDRSLLGSRDSEIGVLIEDKELVDSLMGGKPRKAGKFTLSLRLSLWSEHLGLHSKAINKVIDPVIDSTYKDIWMSTAKTNTMIYQDVFSCVPNDLIHTRAALRQSMVSRKDRLGHTTIDLGIAPQKLESYQNGDIKNTDPLERLQSTRGHLVSFPLEFMCKEDLRPVFNESEYYASQVFH

SEQ ID NO: 7 - DNA, 3291 bp, Artificial Sequence
  Synthesized
atggcatctgagcagttgatgtctcccgccagtggtggtggacgctactttcagatgcagcctgagcaatttccttcgatggtctcttcgctcttctctttcgcgccggctcctacgcaggagactaatcgtatttttgaagaattaccaaaagcagtgatcgtctctgtctctcgccctgatgccggcgatattagccctgtactcttgtcttacaccattgagtgccaatacaagcagttcaagtggcagcttgtgaagaaagcatctcaagtcttttatttgcattttgcattgaagaaacgtgcttttattgaagaaattcacgagaagcaggaacaggttaaagaatggcttcaaaatctaggaataggggatcatccacccgttgtgcaagatgaagatgctgatgaagttccgctacatcaagatgaaagtgccaaaaatagagatgttccttcgagcgctgctttgccagtcattcgtcctttgggaagacagcagtccatatcagttagaggaaagcatgcaatgcaagaatatctgaatcattttctggggaatcttgatatcgtcaattcacgggaggtttgcaggtttttggaggtttcgatgttgtcattctcaccagagtatgggcccaaattgaaagaagactatatcatggtaaaacatctaccgaagttttcaaagagtgatgatgattctaatagatgttgtgggtgctgttggttctgttgctgcaacgataattggcaaaaggtgtggggggtactaaagccaggttttctcgccttattggaagatccatttgatgcgaagctattagatataattgtttttgatgtcctaccagtttctaatggaaatgatggtgtggatatatcactagcagtagaactgaaggatcataatcctttgcggcatgcattcaaggtaacatctggaaaccggagtataagaataagggcaaagaatagtgcaaaagttaaagattgggtggcttctattaacgatgctgctcttagacctcctgagggttggtgccatccccatcgctttggctcatatgctccgccgaggggtttgacggatgacggaagtcaagcccagtggtttgtagatggtggagcagcttttgcagccattgctgcagcgattgaaaatgctaaatctgagattttcatctgtggctggtgggtgtgcccagaactctatcttaggcgtccttttgacccgcatacttcatccagacttgataacttgttggagaataaagctaagcaaggagttcagatatacatccttatctacaaggaggttgctcttgctttaaagatcaacagtgtatatagcaaacgcaggcttcttggcattcatgagaatgtgcgggtacttcgttatcctgatcatttctcaagtggtgtctacctctggtctcaccatgaaaaactcgtcatcgtcgataatcaggtttgctttatcggagggctagacttgtgttttggccgatatgacacgtttgaacataaagttggagataacccttctgtgacatggcctggaaaggactattacaaccccagagagtctgaacccaatacttgggaggatgctctgaaagatgaattagagcgtaaaaagcatccacggatgccttggcatgatgtgcattgtgctttatggggaccaccttgccgtgatgtggctaggcactttgttcaacgctggaactatgctaagagaaacaaagcaccatatgaggattcaattccgcttcttatgcctcaacatcacatggttataccccactacatgggaaggcaagaggagtcagacattgaaagcaagaaagaggaagacagtattagagggattagaagagatgattcattttcttctagatcatctttgcaggacattccattacttttgcctcacgaaccagttgatcaggatggttcgagtggggggcataaagaaaatggaacaaacaacagaaatggtcctttctctttccggaaatcaaaaattgaaccagttgatggagatactcctatgaggggctttgtagatgatcgtaatgggctagatcttccagtagcaaagcgtggttctaatgcaatagattcagagtggtgggaaacacaagatcatgattatcaggttgggtcgccagatgagactgggcaagtcggtccgagaacttcatgccgctgtcagattatacgaagtgtcagtcagtggtctgccggtacaagccaagttgaagagagtatccattctgcttaccgttctctcattgacaaagctgaacattttatctacattgagaatcagtttttcatatcaggcctttctggagatgacacagtaaagaaccgtgtcttagaagcattgtacaagaggattttgcgtgcccataacgagaagaaaattttcagggttgttgttgttatacctctcctccccggtttccagggaggtattgacgacagtggtgcagcatctgttagagccataatgcattggcagtatcgaaccatatacagaggacataactcaatattgactaatctttacaatactattggcgtaaaggctcatgattatatttccttctatggccttagggcatatggtaaactttctgaggatggacctgtcgccactagtcaggtgtatgttcacagtaaaatcatgatagttgatgaccgtgctgcattgattggatctgccaatattaacgaccggagtttgcttggctcaagagattctgagattggagtactaatcgaagacacagagttagtagattctcgcatggcaggaaaaccatggaaggctggaaaattttcttcaagtcttaggctctctttgtggtccgaacaccttggacttcgtactggagagatcgaccagattattgatcccgtctctgattcaacctacaaggagatatggatggcaaccgcaaagacaaacacaatgatataccaggatgtcttctcttgtgtgcccaatgatctcatccattcaagaatggccttcagacaaagcctatcgtattggaaagagaagctgggacacacaacgatcgatttgggaatagcaccagagaagctggagtcttaccacaatggagacatcaagagaagcgatccaatggacagactaaaggcgataaaaggacatctcgtctctttccctttagatttcatgtgcaaagaagatctaagaccggtcttcaatgagagtgaatactacgcctcccctcaagtcttccattga

SEQ ID NO: 8 - DNA, 3141 bp, Artificial Sequence
  Synthesized
atgtcgacggataaattactacttcctaacggcgttaagtcagacggagtcatcagaatgaccagagctgatgctgcggcggcggcagcttcttcttctctcggcggtggaagtcaaatattcgacgagcttcccaaggctgcgatcgtctcggtctcgagacctgacaccaccgattttagtcccttgcttctttcttacaccttggagcttcagtataaacagttcaagtggacattacaaaagaaggcttctcaagttctgtacttacattttgcgttgaagaaacgtttgatcattgaagaacttcacgacaagcaagaacaggttagagagtggctacacagcttggggatttttgatatgcaaggatcagttgtgcaagatgatgaagaacctgacgatggtgctcttcctctgcactatactgaagatagtatcaagaacaggaatgttccttcccgtgcagcgcttccaatcattcgtccaacgataggccggtcagagacagttgtagatcgtgggagaaccgcaatgcaaggctacttgagtctctttctagggaacttggacattgtaaactccaaagaggtctgcaagttcctagaagtttctagactctcatttgctagagagtacggttccaagatgaaagaagggtatgtcacagtgaagcacttgagggacgtcccaggttctgatggtgtccgatgctgtcttcctacacactgtctcggtttcttcggaactagctggacaaaggtttgggcggttctgaaaccaggatttttggcgttactagaagatccattcagcggaaagcttctagatataatggtgttcgacacattggggttgcaaggtactaaagagtcttctgaacaaccgcgtttggctgaacaggtgaaggaacacaacccattgcgttttggctttaaagttactagtggggaccgaaccgtgaggctgagaacaacgagcagcaggaaagttaaagagtgggttaaggccgtggacgaagctggttgttacagtccacatcggtttggttcgtttgcaccacctagaggcttgacatcggacggaagccaggcacagtggttcgtagacggtcacactgcgtttgaagctatcgcgtttgcaatccaaaacgcaacatcagagatatttatgactggttggtggttatgtccggagctatatctcaaacgcccctttgaagatcatccatcattgcggctcgatgcattgctggagacaaaagcaaaacagggcgttaagatatatattcttctgtataaggaagtccaaatcgcgctgaaaatcaacagcttgtacagcaagaaacggcttcaaaacattcacaagaacgtcaaagttcttcgttatccagaccatctctcctccggcatttacctctggtcgcaccacgagaaaatagtgattgtagattaccaagtttgtttcattggagggttagatctctgttttgggcggtacgatacagcggagcacaagattggagattgccctccttatatatggcctggaaaagattactacaatcctagagaatctgaaccaaattcgtgggaagaaacgatgaaagatgagttagacaggagaaagtacccgcgaatgccgtggcacgatgtccactgcgctctatggggaccgccttgtcgggatgtggctcgacattttgtccagcggtggaaccactctaagagaaacaaggcacctaatgaacagacgattccattgttgatgcctcaccaccacatggttcttcctcactacttgggaactagagagatcgatataatcgcagcggctaaaccagaggaagaccctgacaaacctgtcgttcttgccagacatgactctttctcttctgcctcaccgccccaagaaatccctttgcttctcccacaagaaaccgatgcagatttcgccggcagaggagatctgaagttagacagcggtgcaagacaagatcctggggaaacttcagaggaaagcgatctggacgaggctgtgaacgactggtggtggcagattgggaagcagagtgattgccggtgtcaaataatcagaagtgttagccaatggtctgctgggacgagccagcctgaagatagcattcatagagcttattgttcgcttatccagaacgctgaacattttatctacatagagaaccaattcttcatctccgggctagaaaaagaggacacgatcctaaaccgcgttctagaagcgttatacagacgcattctgaaggctcatgaagagaacaagtgcttccgcgttgtgatcgttattccgctactccctggatttcagggaggtattgatgacttcggagcagccacggttcgagcactgatgcattggcaataccgtacgatctctagagaaggaacttcgattcttgacaaccttaacgctttgctcggtcccaagacgcaagattacatctctttctatggtttgagatcgtacggacggctgtttgaggacggtccaattgccactagccagatttacgtgcatagcaagttaatgattgttgatgaccggatcgcagtgatcggatcttctaatataaacgataggagcttactaggttcacgagactctgagatcggtgttgtgattgaagacaaagaattcgtggaatcttcgatgaacggaatgaagtggatggccgggaagttctcttacagtcttagatgttccttgtggtcagagcatctcggccttcacgccggagagattcagaagatcgaagatccaatcaaagatgcaacatacaaagacttatggatggcaacagctaagaaaaacacggacatctacaaccaagtcttctcgtgcatcccgaatgaacatatacgctcaagagctgcattgagacacaatatggctctttgtaaagacaagttgggtcacactacgatcgaccttggcattgcaccggagaggctagaatcatgcggcagcgactcgtgggagattctgaaggagacaagagggaaccttgtgtgcttcccattacagttcatgtgtgatcaagaagatctcagaccaggtttcaacgaatctgagttctacactgctcctcaagtcttccactaa

SEQ ID NO: 9 - DNA, 7942 bp, Artificial Sequence
  Synthesized
cccgacttttgaattttccctcagatttgtcttcctttgtaatccattttcgctctggatcgatcttcctacgatttggtcttcagattgatcggtttctcatcggaattcccgggggggagtataagcataacgaaaaggatcaaactcaggatcaacgcgacgggagaattggagattttttgtttgctagatcttgaattgagcgatcgaattgggtaattggaggtttgaagagatcagagaaatggcatctgagcagttgatgtctcccgccagtggtggtggacgctactttcagatgcagcctgagcaatttccttcgatggtctcttcgctcttctctttcgcgccggctcctacgcaggagactaatcgtatttttgaagaattaccaaaagcagtgatcgtctctgtctctcgccctgatgccggcgatattagccctgtactcttgtcttacaccattgagtgccaatacaagcaggcgagtccatctcttctctttagtaggtcttactaatatcgtcgcataatagatgcttggatcagaatcacttaagaggagatgggtatcaactgtaaattgtcatgtctgactattgatcgaagtagttcttttggatacctagcttgaatctgataccactgatcgatgttatatgtcctttggaagcccggaacagagacatatttatcaatatttatatgtttcaccttagtttagtggaaaggctgcttattatagtggagctttcgacttttgaatacttgagtcctaactttagcgcttttatgtaacagttcaagtggcagcttgtgaagaaagcatctcaagtcttttatttgcattttgcattgaagaaacgtgcttttattgaagaaattcacgagaagcaggaacaggtttgtggtgacttgatatatcaaggaattttcttctactagtttgtgattccctttgagtgaatattttaaaggtttttccttggtctgttgaattgagtttgatgtggttttctttcgaatatcacattaaaggttaaagaatggcttcaaaatctaggaataggggatcatccacccgttgtgcaagatgaagatgctgatgaagttccgctacatcaagatgaaagtgccaaaaataggtcacacttgctggcatttttttttgcttggataagttgctgatgtgagtctatactcttccaacagatgccatcattttctgacaagtagactgttttgttcagagatgttccttcgagcgctgctttgccagtcattcgtcctttgggaagacagcagtccatatcagttagaggaaagcatgcaatgcaagaatatctgaatcattttctggggaatcttgatatcgtcaattcacgggaggtatatttgtaaaatctatttcacttggttgttcaactactttaaaattgttaatacctagttatgtcaagggttgttttctcttctattctagctagaaatcttagtacagcaagtgactatgcgaaaatttccatgcttttcaaacttgtattcctgtacacattgatcgtcattccatttccgaggctatatgcatgcatagttaaatgctaaatgcctaatataggtttgcaggtttttggaggtttcgatgttgtcattctcaccagagtatgggcccaaattgaaagaagactatatcatggtaaaacatctaccgaagttttcaaagagtgatgatgattctaatagatgttgtgggtgctgttggttctgttgctgcaacgataattggcaaaaggtatgaaaattctcattttcttgctcatactcgcgtactatgtattttgctatgaaaaattggtttacctctggagttaactctgggatgacgtgtatgctttgtttatgtgttcttttgttattcttgtccgctctcctactcttgctttgtctctgaactcgttgaatgccatgaatagcttatgtatcagaagaaacaaaattcatctttagtagttcttttgtaatgatttttttttgttactactctcactggagaggtgttctgaaacaaacttatctgaatgcttcaccaccgttgggagatattaagttgatgaagtaaattgtttttctaactctactttagatccatatgttcttcaatgtatgtgccatcgaatactgttgtctccttttataacttctgtctttgcttctgatcagacattctaaattaatgtgacgacaagatggtatctaaggtttctttttctccagtacaggtgtggggggtactaaagccaggttttctcgccttattggaagatccatttgatgcgaagctattagatataattgtttttgatgtcctaccagtttctaatggaaatgatggtgtggatatatcactagcagtagaactgaaggatcataatcctttgcggcatgcattcaaggtcagcaattactgtcctacttatcttttaatcaattatatgtgtgcttactccatctaaataagctttcttgatcatctttttatacaatcctccttgtttgataatctacgatgttgttcttttatgcttcattgcttcttagagaaattaaaataaattgtccaagttatatatccgaattttatttctattacgcatggacactaatgcgccgtggagaactgataaccaagaaaattagaatattccttttgttgtaaattaaaatgggagcctactgaatccttcttttattagctaaaagttgaatcatcctcatttttcttgcccctcttctgctgcacaaatatttctggggttatatgtcaagaaagctatggataatccatgtatataacttttgttaaaatatcccaggtaacatctggaaaccggagtataagaataagggcaaagaatagtgcaaaagttaaagattgggtggcttctattaacgatgctgctcttagacctcctgagggttggtgccatccccatcgctttggctcatatgctccgccgaggggtttgacggatgacggaagtcaagcccagtggtttgtagatggtggagcagcttttgcagccattgctgcagcgattgaaaatgctaaatctgaggtctaccacttttcattactgggtgtggagactgatgatgtgttgaatgttatatattttcaactgatctgatttgccttatagattttcatctgtggctggtgggtgtgcccagaactctatcttaggcgtccttttgacccgcatacttcatccagacttgataacttgttggagaataaagctaagcaaggagttcaggtacttactacagaacatgcatgtacttaatagctattatcttctgttgtgaagcatgtctgtagtctgcaatggagatctgacttgtcaggtttggtggattactatctttcaactttttcatttttattactttctttttcagatatacatccttatctacaaggaggttgctcttgctttaaagatcaacagtgtatatagcaaacgcaggcttcttggcattcatgagaatgtgcgggtacttcgttatcctgatcatttctcaagtggtgtctacctctggtatgatatggtggacagttctaatatatcactctaatatgtcacaatgctctttctagtttatctttctgagtttgatattagataaactgttaaaaaactcctatctgaccacaaaaataagcgggaaggacattgattattacctctgcgtgtctttctaggtctcaccatgaaaaactcgtcatcgtcgataatcaggtttgctttatcggagggctagacttgtgttttggccgatatgacacgtttgaacataaagttggagataacccttctgtgacatggcctggaaaggactattacaaccccaggtaagaatttccttctttatgcgaataaagagttgatttttagatgtaataaaaactaagaacttatgtgtcacttgctagttatttttagctgtgatgaaatcaaattttgtattaaatacaaaccaggatgtccattagtcatttcacctttccgaattcaaccctctgattttttggtttgaacgtacttaaaattttccagagagtctgaacccaatacttgggaggatgctctgaaagatgaattagagcgtaaaaagcatccacggatgccttggcatgatgtgcattgtgctttatggggaccaccttgccgtgatgtggctaggcactttgttcaacgctggaactatgctaaggttcacccatacttctttaactttcttctatctgtttgtttttcctgttattaccgtaacataaccattgtgtttttcagagaaacaaagcaccatatgaggattcaattccgcttcttatgcctcaacatcacatggttataccccactacatgggaaggcaagaggagtcagacattgaaagcaagaaagaggaagacagtattagagggattagaagagatgattcattttcttctagatcatctttgcaggacattccattacttttgcctcacgaaccagttgatcaggatggttcgagtggggggcataaagaaaatggaacaaacaacagaaatggtcctttctctttccggaaatcaaaaattgaaccagttgatggagatactcctatgaggggctttgtagatgatcgtaatgggctagatcttccagtagcaaagcgtggttctaatgcaatagattcagagtggtgggaaacacaagatcatgattatcaggttgggtcgccagatgagactgggcaagtcggtccgagaacttcatgccgctgtcaggtgggtgttatgaaagtttatatataccaattatatgtggaatttcaaatctgaatagagctgatgctattatgtacgatcctattctgtttcatgcttggcttcagcatacaagctttaaaattttattgtgatcataataataagaatttcccttcaacatttagcactgattagaactattttcagattatacgaagtgtcagtcagtggtctgccggtacaagccaagttgaagagagtatccattctgcttaccgttctctcattgacaaagctgaacattttatctacattgaggtatttgctataacatgtgtttgtcgtggaatctcggttttgcatgagttcaatttgatagaccaagtaattagagcatcatttagcgtttcattgaaacgacctgacatcttttctccctgaattatgcataatgcaatttgcatcacattacattcaacttgggagaaatatggaacattctgctaaatgtgctggtaatttgagctgtcagtcaaataatgtatacatgtattggtttccgtactttgatttggcacctctctgattccctcttagccttggtgagctttgctggagatcctgcagctttccttggtcaccttttatctggcctacacaaaagtttaatttatctggtgaaagaaactaatcccctattgaacgtggacaaactaagcattaagtaatagtcctatacccttgctcctgcaaaatccaccttgcaaatcattcatactttatatttcagataatacaagtaacttagttcttgaaactgtagtagttgccaggttcacattgtctttatttttctgacttctgtttgcatgttgtgcctccagaatcagtttttcatatcaggcctttctggagatgacacagtaaagaaccgtgtcttagaagcattgtacaagaggattttgcgtgcccataacgagaagaaaattttcagggttgttgttgttatacctctcctccccggtttccaggttagtttcggctagaaaagtgtattctgtttaaaaagatttgctcctgcaatgcaactttttaaaccaacgtatgtgtctggcagggaggtattgacgacagtggtgcagcatctgttagagccataatgcattggcagtatcgaaccatatacagaggacataactcaatattgactaatctttacaatactattggcgtaaaggctcatgattatatttccttctatggccttagggcatatggtaaactttctgaggatggacctgtcgccactagtcaggtaacaactcaatcatccagggtttagatttgtgcattgctgcctttttttttgtgatcacggaatcatataggatgccctgttatacatgtgcttgaatgcagtggtagtgagtattaaaaatgtgaatgctatttttttctctcaatgcaggtgtatgttcacagtaaaatcatgatagttgatgaccgtgctgcattgattggatctgccaatattaacgaccggagtttgcttggctcaagagattctgaggtcatattttactcacgtactttcttgtgctaagcaatgatttccaaaagcctatttcatacttgaaactaaggtgaagatcataatagaaaacaaaaagagtaaaaaaaattatttcctctgttgaaggaaaaagattttcgttgcctcagcacgttatatcagagtttatagttaggaataaattcatttaaagtatgttgaacaagtttttttatataactgtgtatgctgaacataaaaaagcataaaatgcttctgtatgctttatatcagattggagtactaatcgaagacacagagttagtagattctcgcatggcaggaaaaccatggaaggctggaaaattttcttcaagtcttaggctctctttgtggtccgaacaccttggacttcgtactggagaggtttgtgtcatattctctataatgttcttacgaagtggtctaagatatttcagtatataggcagatttaacacttgaaaagccatggaaattattcttttacttagacttaatgatgaaagatgatctgaaccacgccggcatgaactttcaatcaatggttatgactgaagaggatcttaagactataaatgtgctttgattactaaaatgtgatctttctttgtagatcgaccagattattgatcccgtctctgattcaacctacaaggagatatggatggcaaccgcaaaggttggaaacgtgattacagagagaaatttatattttcacttaccagatataaccagtgagcttatttatcccaacgcagacaaacacaatgatataccaggatgtcttctcttgtgtgcccaatgatctcatccattcaaggtacaattccggtctatcttttctttcttcacctttggccactatcattattccttttgaaacgaaaagattccggattatgattaatccatggcattggatcgtaaaatctcttgaatatatacgcagaatggccttcagacaaagcctatcgtattggaaagagaagctgggacacacaacgatcgatttgggaatagcaccagagaagctggagtcttaccacaatggagacatcaagagaagcgatccaatggacagactaaaggcgataaaaggacatctcgtctctttccctttagatttcatgtgcaaagaagatctaagaccggtcttcaatgagagtgaatactacgcctcccctcaagtcttccattgagatcattacatcaattcttgactgctggcttattcagtgacagctctctacacgaagaatacttcattgctacctttaaccattttcatctcatatgtttatgagaaagaattaaagatgtttgtatagcgatatgatatgtgcaaacagaggaggaaactgagaagggaagccattttgctagtttttggtttctctcttccgaggtaaagtaaaaaaagaaaagaaacaggtcattttctaagtcaaatattggcttactatctataacagtgaatgtgtcaccccacacagacacatatgatttgtaattatgcgaaaaaagac

SEQ ID NO: 10 - DNA, 5149 bp, Artificial Sequence
  Synthesized
aataaaatctaaaatcaatagacacctatataacgtcgatttattgatcagtggaatctcgagtatttcggacatgcaatcgtgaacggcgagtttcgacgacggtttggggagttaatcgatgtcgacggataaattactacttcctaacggcgttaagtcagacggagtcatcagaatgaccagagctgatgctgcggcggcggcagcttcttcttctctcggcggtggaagtcaaatattcgacgagcttcccaaggctgcgatcgtctcggtctcgagacctgacaccaccgattttagtcccttgcttctttcttacaccttggagcttcagtataaacaggtatatatatagtttacaatgtctgacttgtagttttaaaagaacttgatttcctctctgttacattgtgattagggttttgattttgtctatgtatacgaaatttatgacaaagagatgccattaagagattcaatttcttgctaaagaagtttattcatgcatatatgaaatttctagtggttgtttctctaaaagtttatgttctgtttttattagtaacaaacaaagaccttttttaattggtattctctgtttttggtatatgtatagttcaagtggacattacaaaagaaggcttctcaagttctgtacttacattttgcgttgaagaaacgtttgatcattgaagaacttcacgacaagcaagaacaggtcagaatgttcaggctttgcagatttgctactaacacaagccaaacaaataattattgaactccagagatgcggttttcgacaggttagagagtggctacacagcttggggatttttgatatgcaaggatcagttgtgcaagatgatgaagaacctgacgatggtgctcttcctctgcactatactgaagatagtatcaagaacaggtgcttttaaatgccataaaacttttcggttttgtggattcaaagtaaaaattgtttcgtttaaatttctgaaacagtagctatttatgttatgcaggaatgttccttcccgtgcagcgcttccaatcattcgtccaacgataggccggtcagagacagttgtagatcgtgggagaaccgcaatgcaaggctacttgagtctctttctagggaacttggacattgtaaactccaaagaggtttgagatttttcactggcgatgactaagacttttctagttgtggtttttagatttcttgcgtcacacttaaaacgtattcacaggtctgcaagttcctagaagtttctagactctcatttgctagagagtacggttccaagatgaaagaagggtatgtcacagtgaagcacttgagggacgtcccaggttctgatggtgtccgatgctgtcttcctacacactgtctcggtttcttcggaactagctggacaaaggtttggtcaaaatcaactcggggtttttgtaaagtttctgaatttttcttgctcagatgaatgattttctgcatttttgaatgttttcaggtttgggcggttctgaaaccaggatttttggcgttactagaagatccattcagcggaaagcttctagatataatggtgttcgacacattggggttgcaaggtactaaagagtcttctgaacaaccgcgtttggctgaacaggtgaaggaacacaacccattgcgttttggctttaaagttactagtggggaccgaaccgtgaggctgagaacaacgagcagcaggaaagttaaagagtgggttaaggccgtggacgaagctggttgttacagtccacatcggtttggttcgtttgcaccacctagaggcttgacatcggacggaagccaggcacagtggttcgtagacggtcacactgcgtttgaagctatcgcgtttgcaatccaaaacgcaacatcagaggttccatttctttgcctcaacttgtatttgagaatcgtaccatataatatcttagttttaaatctaaaatttcgtctacagatatttatgactggttggtggttatgtccggagctatatctcaaacgcccctttgaagatcatccatcattgcggctcgatgcattgctggagacaaaagcaaaacagggcgttaaggtatgcgtaatgagcttacatttctctcgaaaggattcgatttgttttcttatgtaagcgtacactaaaaatttaaaagttgttgcagatatatattcttctgtataaggaagtccaaatcgcgctgaaaatcaacagcttgtacagcaagaaacggcttcaaaacattcacaagaacgtcaaagttcttcgttatccagaccatctctcctccggcatttacctctggtacagttttcatcgtttcagggatttacttaacaactacttagctataactaaatttgaattctgtggttttttcaggtcgcaccacgagaaaatagtgattgtagattaccaagtttgtttcattggagggttagatctctgttttgggcggtacgatacagcggagcacaagattggagattgccctccttatatatggcctggaaaagattactacaatcctaggtaatatcctgaactttttattatcttttctaacaaaaggaatcatctttctcacaaattgcttctattgtcagagaatctgaaccaaattcgtgggaagaaacgatgaaagatgagttagacaggagaaagtacccgcgaatgccgtggcacgatgtccactgcgctctatggggaccgccttgtcgggatgtggctcgacattttgtccagcggtggaaccactctaaggtagagagatttcagtttttagtgtttgcatttggaaatcatggttctgatatattttagtctttgttatattagagaaacaaggcacctaatgaacagacgattccattgttgatgcctcaccaccacatggttcttcctcactacttgggaactagagagatcgatataatcgcagcggctaaaccagaggaagaccctgacaaacctgtcgttcttgccagacatgactctttctcttctgcctcaccgccccaagaaatccctttgcttctcccacaagaaaccgatgcagatttcgccggcagaggagatctgaagttagacagcggtgcaagacaagatcctggggaaacttcagaggaaagcgatctggacgaggctgtgaacgactggtggtggcagattgggaagcagagtgattgccggtgtcaaataatcagaagtgttagccaatggtctgctgggacgagccagcctgaagatagcattcatagagcttattgttcgcttatccagaacgctgaacattttatctacatagaggtataaattaatcattcaaacatttttatacaaaatctctacacctcagattggttctatattctaatctcttaatcttgttgtttaacagaaccaattcttcatctccgggctagaaaaagaggacacgatcctaaaccgcgttctagaagcgttatacagacgcattctgaaggctcatgaagagaacaagtgcttccgcgttgtgatcgttattccgctactccctggatttcaggtactgtttgcttcttctcaattacaaattgacaaattgttaacagagtataatcatgatgtgtcgacataaaacagggaggtattgatgacttcggagcagccacggttcgagcactgatgcattggcaataccgtacgatctctagagaaggaacttcgattcttgacaaccttaacgctttgctcggtcccaagacgcaagattacatctctttctatggtttgagatcgtacggacggctgtttgaggacggtccaattgccactagccagatttacgtgcatagcaagttaatgattgttgatgaccggatcgcagtgatcggatcttctaatataaacgataggagcttactaggttcacgagactctgaggtactttcaaaaatccaattcattctttattgcagcaaaacagagttatgtattcatttgaatcaatcatgtttcagatcggtgttgtgattgaagacaaagaattcgtggaatcttcgatgaacggaatgaagtggatggccgggaagttctcttacagtcttagatgttccttgtggtcagagcatctcggccttcacgccggagaggtaattttaaaaaatttctagaaacgcctactactatacatttttgacttcagaaacctttattttcatttgagtatatatagccgaaaactttttagtatttttacttgtttttgctctgttctctgatagtcttggtatcctatttccgttggataagattccttgagctgaaagcttcttttaactaccgttacctccatagaaaatttgaaaatgttgattttgttttttgtgtttagattcagaagatcgaagatccaatcaaagatgcaacatacaaagacttatggatggcaacagctaagaaaaacacggacatctacaaccaagtcttctcgtgcatcccgaatgaacatatacgctcaaggttcatacgtcatccctttttaactaaatctccataactttagtgattaaagatacgtaactacgttggctttttaaacgcaactgagagatgatcaaatctgcttctgactttgtctttctaaacacagagctgcattgagacacaatatggctctttgtaaagacaagttgggtcacactacgatcgaccttggcattgcaccggagaggctagaatcatgcggcagcgactcgtgggagattctgaaggagacaagagggaaccttgtgtgcttcccattacagttcatgtgtgatcaagaagatctcagaccaggtttcaacgaatctgagttctacactgctcctcaagtcttccactaaccactatttattgtacgcccagttctctttaatcagttaatagagtacctaagctcacacgttacttatgtatagagatgttagttatatagaaagaagaaattcatttgattgcttcctaggttcgcagaggtatgtgtgtgtatagtatacacttcttgtaaatcataatgtttatgtgcctcaagctgtacaatacatcttcaattgcggatatgttcttacaactttggagttgatata

SEQ ID NO: 11 - DNA, 31 bp, Artificial Sequence
  Synthesized
cgggcggccgcggaagacttgaggggaggcg

SEQ ID NO: 12 - DNA, 31 bp, Artificial Sequence
  Synthesized
cgggcggccgcagagaaatggcatctgagca

SEQ ID NO: 13 - DNA, 31 bp, Artificial Sequence
  Synthesized
cgggcggccgcagtggaagacttgaggagca

SEQ ID NO: 14 - DNA, 31 bp, Artificial Sequence
  Synthesized
cgggcggccgcgacgacggtttggggagtta

SEQ ID NO: 15 - DNA, 29 bp, Artificial Sequence
  Synthesized
cggggtacccgcgccaagcttttgatcca

SEQ ID NO: 16 - DNA, 29 bp, Artificial Sequence
  Synthesized
cgcggatccggaagacttgaggggaggcg

SEQ ID NO: 17 - DNA, 29 bp, Artificial Sequence
  Synthesized
cgcggatccggtggaagacttgaggagca

SEQ ID NO: 18 - DNA, 32 bp, Artificial Sequence
  Synthesized
gcgggcgcgcccgcgccaagcttttgatccat

SEQ ID NO: 19 - DNA, 25 bp, Artificial Sequence
  Synthesized
atggcgcgccagtcacgacgttgta

SEQ ID NO: 20 - DNA, 23 bp, Artificial Sequence
  Synthesized
ggttcatcttctgcattttcgga

SEQ ID NO: 21 - DNA, 23 bp, Artificial Sequence
  Synthesized
ttttcggttcatcaggtcgtggt

SEQ ID NO: 22 - DNA, 23 bp, Artificial Sequence
  Synthesized
tgtttgagaggcacaagtcccga

SEQ ID NO: 23 - DNA, 23 bp, Artificial Sequence
  Synthesized
agtccaaatccagctccaaggta

SEQ ID NO: 24 - DNA, 23 bp, Artificial Sequence
  Synthesized
ggagtggggataccaacggaacg

SEQ ID NO: 25 - DNA, 23 bp, Artificial Sequence
  Synthesized
gaaaggggatgcaactgtcggga

SEQ ID NO: 26 - DNA, 23 bp, Artificial Sequence
  Synthesized
ctaagatgatgagacgagccgaa

SEQ ID NO: 27 - DNA, 23 bp, Artificial Sequence
  Synthesized
atctctcggtcggaatccctact

SEQ ID NO: 28 - DNA, 20 bp, Artificial Sequence
  Synthesized
gccacttctactaaactccc

SEQ ID NO: 29 - DNA, 20 bp, Artificial Sequence
  Synthesized
cacacacaaacaaacacatc

SEQ ID NO: 30 - DNA, 20 bp, Artificial Sequence
  Synthesized
ctgacgatttccaaagacaa

SEQ ID NO: 31 - DNA, 20 bp, Artificial Sequence
  Synthesized
ttcaatccctttgttgctca

SEQ ID NO: 32 - DNA, 20 bp, Artificial Sequence
  Synthesized
gcccttggaatctactgctt

SEQ ID NO: 33 - DNA, 20 bp, Artificial Sequence
  Synthesized
acataacttcacctatcctg

SEQ ID NO: 34 - DNA, 20 bp, Artificial Sequence
  Synthesized
cgaaccaaaaggattgaaaa

SEQ ID NO: 35 - DNA, 20 bp, Artificial Sequence
  Synthesized
tccacaagaggaaccccgtc

SEQ ID NO: 36 - DNA, 22 bp, Artificial Sequence
  Synthesized
tggatggcaaccgcaaagacaa

SEQ ID NO: 37 - DNA, 22 bp, Artificial Sequence
  Synthesized
atcgttgtgtgtcccagcttct

SEQ ID NO: 38 - DNA, 22 bp, Artificial Sequence
  Synthesized
tttgaggacggtccaattgcca

SEQ ID NO: 39 - DNA, 24 bp, Artificial Sequence
  Synthesized
acaacaccgatctcagagtctcgt

SEQ ID NO: 40 - DNA, 22 bp, Artificial Sequence
  Synthesized
cacactccacttggtcttgcgt

SEQ ID NO: 41 - DNA, 24 bp, Artificial Sequence
  Synthesized
tggtctttccggtgagagtcttca